(19) United States
(12) Patent Application Publication
(10) Pub. No.: US 2026/0191687 A1
(43) Pub. Date: Jul. 9, 2026

(54) LASER SYSTEM HAVING AN ORIENTATION ANALYSIS SYSTEM FOR MEASURING THE ORIENTATION OF THE PATIENT

(71) Applicant: c/o TECHNOLAS PERFECT VISION GMBH, München (DE)

(72) Inventors: Rui LIU, München (DE); Wolfgang KÜGEL, München (DE); Georg HENNIG, München (DE); Christian WÜLLNER, München (DE); Christof DONITZKY, München (DE)

(21) Appl. No.: 19/559,046

(22) Filed: Mar. 6, 2026

Related U.S. Application Data
(63) Continuation of application No. PCT/EP2024/075045, filed on Sep. 6, 2024.

(30) Foreign Application Priority Data
Sep. 7, 2023 (EP) 23196088.1

(51) Int. Cl. A61F 9/008; A61B 90/00; A61B 90/50

(52) U.S. Cl. CPC A61F 9/008; A61B 90/50; A61B 2090/0807

(57) ABSTRACT

The disclosure relates to a guide configured to allow moving at least a portion of the beam delivery system, to adjust an orientation of at least the movable portion to different orientations of the patient's head measured around an axis of the eye to be treated. The system has an orientation analysis system for acquiring orientation-related data using one or more body portions of the patient. The ophthalmic laser system allows a user and/or the controller, using the orientation-related data, to move the movable portion of the beam delivery system so that during the laser treatment, the movable portion has an orientation around the axis of the eye and relative to the patient's head, which corresponds or substantially corresponds to a pre-determined target orientation; or which is within or substantially within a pre-determined target range.

### CROSS-REFERENCE TO RELATED APPLICATIONS

This application is a Bypass Continuation Application of PCT Application Serial Number PCT/EP2024/075045 filed on Sep. 6, 2024, which application claims the benefit of and priority to European Application No. 23196088, filed Sep. 7, 2023, the entire contents of each of which are hereby incorporated herein by reference. This application is related to U.S. patent application Ser. No. 18/826,436, entitled “LASER SYSTEM HAVING AN ORIENTATION ANALYSIS SYSTEM FOR MEASURING THE ORIENTATION OF THE PATIENT,” filed on Sep. 6, 2024.

### FIELD

The present invention relates to a system for performing laser treatments, such as treatments which form incisions within the cornea or the natural lens of the eye. The present invention also relates to systems for ablating a surface portion of the cornea, such as photorefractive keratectomy (PRK) and laser in situ keratomileusis (LASIK) treatments.

### BACKGROUND

Cataract surgery is one of the most common ophthalmic surgical procedures performed. The primary goal of cataract surgery is the removal of the aging lens and replacement with an artificial lens or intraocular lens (IOL) that restores some of the optical properties of the aged lens.

The major steps in cataract surgery consist of forming a corneal incision to allow access to the anterior chamber of the eye and incisions to correct for astigmatism (Limbal relaxing incisions, abbreviated as LRIs or astigmatic keratotomy, abbreviated as AK), cutting and opening the capsule of the lens to gain access to the lens (capsulotomy), lens fragmentation and removing the fragmented lens through phacoemulsification, irrigation and aspiration and in most cases placing an artificial intraocular lens in the eye.

Therefore, femtosecond laser-assisted cataract surgery is a multi-stage process which includes femtosecond laser surgery (LRI, AK, corneal incision, capsulotomy and lens fragmentation), a phacoemulsification process (emulsifying, liquifying and aspirating the lens) and manual surgical procedures (implantation of the intraocular lens).

Using conventional femtosecond laser systems, this, however, typically requires that the patient is moved from his regular patient bed to a separate stationary patient support which is part of the femtosecond laser system and which is specifically adapted for conducting the laser surgical steps. Such patient transfer procedures, however, complicate the clinical workflow and are uncomfortable for the patients.

Moreover, in order to reduce footprint and costs, it is desirable that femtosecond laser systems, which are configured for cataract surgery also be used for forming corneal flaps for laser in situ keratomileusis (LASIK). A femtosecond laser for forming LASIK flaps, however, should be located close to the excimer laser system in order to make it easy for the patient to move from one system to the other. On the other hand, a femtosecond laser for cataract surgery should be located in the sterile operating theatre, where phacoemulsification and IOL implantation are carried out.

In view of the above, there is a need to provide a laser system which allows an efficient clinical workflow.

### SUMMARY

Embodiments of the present disclosure pertain to an ophthalmic laser system configured to perform laser treatments on an eye of a patient using a treatment laser beam. The laser system comprises a beam delivery system for focusing the treatment laser beam so that the treatment laser beam travels toward the eye to be treated. The laser system further comprises a controller operatively coupled with the beam delivery system and configured to control the beam delivery system to position a focus of the laser beam at different locations within the eye or on an anterior surface of the cornea of the eye. The laser system further comprises a guide configured to allow moving at least a portion of the beam delivery system, to adjust an orientation of at least the movable portion to different orientations of the patient's head measured around an axis of the eye to be treated. The laser system further comprises an orientation analysis system for acquiring orientation-related data using (a) one or more body portions of the patient; and/or (b) one or more objects, each of which having a fixed position and/or orientation relative to a respective body portion so that the orientation-related data depend on an orientation of the one or more body portions around the axis of the eye. The ophthalmic laser system is configured to allow a user and/or the controller, using the orientation-related data, to move the movable portion of the beam delivery system so that during the laser treatment, the movable portion has an orientation around the axis of the eye and relative to the patient's head, which (i) corresponds or substantially corresponds to a pre-determined target orientation; or which (ii) is within or substantially within a pre-determined target range.

The term “axis of the eye” as used herein may refer to the visual axis of the eye or to the optical axis of the eye. The term “optical axis of the eye” as used herein may refer to an axis, which is oriented normal to the anterior surface of the cornea and which has the smallest distance from the center of curvature of all remaining refractive surfaces of the eye. The term “visual axis of the eye” (also referred to as “line of vision”) as used herein may refer to the straight line extending from the object seen, through the center of the pupil, to the macula lutea of the retina.

At least one of the one or more objects may cover and/or contact, in particular directly contact a corresponding body portion relative to which the object has a fixed (i.e. unchanged) position and/orientation.

Each of the body portions, which are analyzed, either directly or using the one or more objects, may represent a structural and/or anatomical portion of the patient's head, such as at least a portion of an eye, an ear, and/or a nose of the patient. Therefore, the body portions may function as an indicator for the position and/or orientation of the patient's head.

The controller may include a data processing system. The data processing system may include a computer system having one or more processors and a memory for storing instructions processable by the processor. The one or more processors may execute an operating system. The data analysis system may further include a user interface configured to allow a user to receive data from the data processing system and/or to provide data to the data processing system. The user interface may include a graphical user interface. The ophthalmic laser system may include a pointing device, such as a computer mouse. The graphical user interface may be configured for receiving user input via the pointing device.

The guide may include one or more rotary guides and/or linear guides. Each of the linear guides may be configured as a straight or curved linear guide. In a configuration, where the position and/or orientation of the movable portion of the beam delivery system is adjusted relative to the patient in order to perform the laser treatment, a rotation axis of the guide may extend in a vertical or substantial vertical direction. During the laser treatment, the patient's eye may be docked to the movable portion of the beam delivery system via a contact element of a patient interface, wherein the contact element contacts an anterior surface of the cornea of the patient's eye and the treatment laser, traverses the contact element after having exited the beam delivery system.

The guide may be configured for motorized adjustment of a position and/or orientation of the movable portion of the beam delivery system, in particular for a motorized adjustment of the orientation about the axis of the eye to be treated.

The guide may be configured to provide one, two or three rotary degrees of freedom for positioning the movable portion of the beam delivery system relative to the patient's eye. By way of example, the guide my include one, two or three rotational bearings and/or a spherical bearing. The rotation axes of the rotational bearings may be mutually oriented non-parallel with respect to each other.

Additionally or alternatively, the guide may be configured to provide one, two or three translational degrees of freedom. Specifically, the guide may include one, two, or three linear guides, which are mutually oriented non-parallel with respect to each other. Each of the linear guides may be configured as a straight or a curved linear guide.

The ophthalmic laser system may be configured so that the movement of the movable portion, using the orientation-related data, may be performed, at least in part, manually by the user based on data which is provided to the user via a user interface of the ophthalmic laser system. By way of example, the data may be indicative of a current orientation angle of the orientation of the patient's head relative to the movable portion of the beam delivery system. The data may be determined by the controller using the orientation-related data. Additionally or alternatively, the ophthalmic laser system may include a drive system for moving the movable portion of the beam delivery system. The controller may be operatively coupled to the drive system. The controller may be configured to control the drive system, based on the orientation-related data in order to perform the movement of the movable portion of the beam delivery system.

The beam delivery system may include an objective lens system for focusing the treatment laser beam within the eye. At least a portion of the objective lens system may be part of the movable portion of the beam delivery system.

The beam delivery system may include a scanning system, which is operatively coupled with the controller. The scanning system may provide three degrees of freedom for positioning the focus of the laser beam at different locations within the eye or on the anterior surface of the cornea. One of the three degrees of freedom may be provided by the axial scanning system. The remaining two degrees of freedom of the scanning system may be provided by a beam deflection scanning system.

The axial scanning system may be configured for scanning the laser focus along an axis of the laser beam. The beam deflection scanning system may be configured for scanning the laser beam through deflection of the laser beam. The axial scanning system may be in the beam path of the laser beam between a laser source of the laser system and the beam deflection scanning system. The scanning system may be in the beam path of the treatment laser beam between the laser source and the focusing optical system.

In addition to a scanning movement of the laser focus along the axis of the laser beam, the axial scanning system may also cause a deflection of the laser beam so that the focus of the laser beam performs a lateral movement concurrently with the axial movement. The lateral movement of the laser focus may be smaller than the axial movement of the laser focus. A deflection of the laser beam, which is performed using the beam deflection scanning system may adjust a lateral position of the laser focus relative to an optical axis of the beam delivery system.

The axial scanning system may be configured to axially scan the focus of the laser beam through varying an angle of divergence or convergence of the laser beam. The divergence or convergence may be measured at a location along the axis of the laser beam, where the laser beam exits from the axial scanning system. The terms “divergence” or “convergence” as used herein may be defined to mean an angular measure of the increase or decrease in beam diameter with distance.

The axial scanning system may include one or more displaceable lenses, which are in the beam path of the treatment laser beam. The axial scanning system may be configured so that the one or more displaceable lenses are controllably displaceable in a direction parallel or substantially parallel to an optical axis of the one or more displaceable lenses. The ophthalmic laser system may include a controller, which is in signal communication with an actuator of the axial scanning system. The actuator may be configured to displace the one or more displaceable lenses based on signals received from the controller.

The axial scanning system may include a first optical system, which has a negative optical power; and a second optical system, which has a positive optical power. The second optical system may be in the beam path of the laser beam between the first optical system and the deflection scanning system. The axial scanning system may further be configured so that a distance between the first optical system and the second optical system is controllably variable.

The ophthalmic laser system may further include an imaging system for acquiring a frontal image of at least a portion of the eye to be treated, at least during a state in which the eye is docked or substantially docked to the ophthalmic laser system. The imaging system may include an image sensor and an imaging optical system for imaging the portion of the eye onto the image sensor. The image sensor may include a two-dimensional ordered or unordered array of pixels. The image sensor may be sensitive to one or more wavelengths within a range of between 380 nanometers and 950 nanometers or within a range of between 380 nanometers and 1400 nanometers.

The beam path of the eye imaging system may traverse a contact element of the patient interface. The contact element may be in contact with an anterior surface of a cornea of the eye to be treated. At least a portion of the imaging beam path of the eye imaging system may traverse at least a portion of the movable portion of the beam delivery system. The optical elements of the movable portion of the beam delivery system may include optical elements of a focusing optical system for focusing the treatment laser beam within the eye and/or an exit optical system through which the treatment laser beam exits from the beam delivery system toward the contact element of the patient interface. The imaging beam path of the eye imaging system may be outside of the scanning system, which is traversed by the treatment laser beam.

The beam delivery system, in particular the movable portion of the beam delivery system, may include a beam combiner, which is in the beam path of the laser beam between the scanning system and the eye. The beam combiner may be configured for combining the beam path of the treatment laser beam with the beam path of the imaging system. The beam combiner may be configured to deflect the treatment laser beam in a direction toward or substantially toward the eye. The beam combiner may include a mirror and/or a prism. The beam combiner may be configured as a dichroic beam combiner. The beam combiner may be in the beam path of the treatment laser beam downstream of the focusing optical system, within the focusing optical system or upstream of the focusing optical system. Downstream of the beam combiner the treatment laser beam may extend or substantially extend in a vertical direction.

The controller may be configured to use images of the imaging system to determine a pupil center of the eye to be treated. The controller may further be configured to use the determined pupil center to align laser positioning data, such as a scanning pattern for scanning the focus of the treatment laser beam within the eye, with the axis of the eye to be treated. The controller may further be configured to use images of the eye imaging system to determine an orientation of the laser positioning data about the axis of the eye to be treated. By way of example, the controller may execute an image analysis algorithm to extract the position and/or orientation of anatomical landmarks and/or anatomical features of the eye to be treated and to compare the image with an images, which has been acquired using a diagnostic device. By way of example, the diagnostic device includes a a wavefront analysis system and/or an OCT system. This allows determination of the laser positioning data based on measurements of the diagnostic device. Examples of anatomical landmarks are but are not limited to: structural features of the iris and conjunctival blood vessels.

The movable portion of the beam delivery system may include at least a portion of (a) the axial scanning system, (b) the deflection scanning system, (c) the eye imaging system and/or a focusing optical system for generating a focus of the treatment laser beam within the eye to be treated.

According to an embodiment, at least one of the body portions is outside the eyeball of the eye to be treated. At least one of the body portions may not only be outside the eyeball of the eye to be treated but also outside of one or more of (a) the upper and/or lower eyelid, (b) the eye lashes, (c) the tear duct, (d) the lacrimal punctum and (e) the lacrimal caruncle of the eye to be treated.

According to an embodiment, the guide includes a rotational guide configured to allow rotation of the movable portion of the beam delivery system about a rotation axis.

The rotation axis may extend parallel or may be aligned with an optical axis of the movable portion of the beam delivery system. The rotational guide may have a single motional degree of freedom. Therefore, the adjustment of the orientation to different orientations of the patient's head about the axis of the eye to be treated can be performed by rotating the movable portion of the beam delivery system about the rotation axis of the rotational guide. However, it is also conceivable that the orientation of the movable beam delivery system about the axis to be treated is adjusted using a plurality of rotational guides.

According to an embodiment, the laser system is configured so that during laser treatment, the rotation axis of the rotational guide is parallel to or aligned with the optical axis of the movable portion of the beam delivery system. The optical axis may be defined by one or more exit optical elements of the beam delivery system through which the treatment laser beam exits from the beam delivery system to travel toward the patient interface. During treatment, the optical axis of the movable portion of the beam delivery system may be aligned or substantially aligned with the axis of the eye to be treated.

According to an embodiment, the controller is configured to determine, automatically or using user input, based on the orientation-related data, one or more parameters, which are (a) indicative of or (b) correlated with one or more, or all, parameters of an orientation of the patient's head around the axis of the eye to be treated.

The ophthalmic laser system may be configured to output to the user, using a user interface of the ophthalmic laser system, graphical and/or textual information, which is generated by the controller using the one or more parameters.

Embodiments of the present invention pertain to an ophthalmic laser system configured to perform laser treatments on an eye of a patient using a treatment laser beam, the laser system. The laser system comprises a beam delivery system for focusing the treatment laser beam so that the treatment laser beam travels toward the eye to be treated. The laser system further comprises a controller operatively coupled with the beam delivery system and configured to control the beam delivery system to position a focus of the laser beam at different locations within the eye or on an anterior surface of the cornea of the eye based on laser positioning data, which are indicative of or correlated with the locations within the eye to be treated. The laser system further comprises an orientation analysis system for acquiring orientation-related data using (a) one or more body portions of the patient and/or (b) one or more objects. Each of the objects is in a fixed position and/or orientation relative to a respective body portion, so that the orientation-related data depend on an orientation of the one or more body portions around the axis of the eye. At least one of the body portions is outside of an eyeball of the eye to be treated. The controller may further be configured to determine, based on the orientation-related data: (i) one or more parameters of the laser positioning data; and/or (ii) whether an exit optical element of the beam delivery system is positioned in front of the left or in front of the right eye of the patient. The exit optical element may be an optical element of the delivery system, through which the treatment laser beam exits from the beam delivery system toward the patient interface and/or toward the eye to be treated.

The laser positioning data may include one or more scanning paths for scanning the focus of the treatment laser beam within the eye. Examples for such scanning paths are but are not limited to: scanning paths within in the cornea, scanning paths for performing anterior capsulotomy (i.e. forming a flap or an opening in the lens capsule), scanning paths for posterior capsulotomy (i.e. for forming an opening in the posterior lens capsule) and/or scanning paths for lens fragmentation.

Examples for scanning paths within the cornea are scanning paths for forming an anterior surface lamella, (such as LASIK flaps), an intrastromal lamella (such as in SMILE and FLEx procedures) or a posterior surface lamella (such as in endothelial keratoplasty procedures). SMILE is an abbreviation for “small incision lenticule extraction”, FLEx is an abbreviation for “femtosecond lenticule extraction” and LASIK is an abbreviation for “laser-assisted in-situ keratomileusis”. Further examples are scanning paths for forming limbal relaxing incisions (LRI) and cataract incisions for providing access to the anterior chamber during cataract surgery.

Determining the laser positioning data based on the orientation-related data may comprise determining an orientation of the locations of the laser positioning data around the axis of the eye based on the orientation-related data, in particular based on one or more or all parameters which are indicative of the orientation of the patient's head about the axis of the eye to be treated.

According to a further embodiment, the controller is configured to determine, based on the orientation-related data, automatically or using user input, one or more parameters, which are indicative of or correlated with one or more, or all, parameters of an orientation of the patient's head.

The orientation of the patient's head may be measured (a) relative to the movable portion of the beam delivery system or (b) relative to a stationary coordinate system or relative to a base of the ophthalmic laser system, which supports the supporting arm and/or which houses the laser source.

According to a further embodiment, the one or more parameters of the orientation of the patient's head are measured around the axis of the eye to be treated. Additionally or alternatively, the one or more parameters of the orientation of the patient's head are measured (i) relative to the movable portion of the beam delivery system; or (ii) relative to a stationary coordinate system.

The orientation analysis system which is configured to measure the one or more parameters relative to the movable portion may be mounted so that at least a portion of the orientation analysis system is mounted at a fixed position and/or orientation relative to the movable portion of the beam delivery system. The orientation analysis system which is configured to measure the one or more parameters relative to the stationary coordinate system may be mounted so that at least a portion of the orientation analysis system is mounted at a fixed position and/or orientation relative to the base of the laser system or relative to a fixed object in the environment, such as a wall or a ceiling of a room (such as an operating room) in which the laser system is located.

According to an embodiment, the determination of the one or more parameters comprises determining, automatically or based on user input, one or more or all parameters of an angular position of an anatomical feature and/or an anatomical landmark relative to the axis of the eye. Additionally or alternatively, the determination comprises determining, automatically or based on user input, one or more or all parameters of an orientation of the anatomical features and/or the anatomical landmark in a plane perpendicular to the axis of the eye.

The term “angular position” as used herein may be understood to mean the azimuth angle in a cylindrical coordinate system. Therefore, the angular position of an anatomical landmark relative to the axis of the eye relates to the azimuth angle in a cylindrical coordinate system in which the axis of the eye corresponds to the cylindrical axis of the cylindrical coordinate system.

The anatomical feature and/or the anatomical landmark may include or may be at least partially defined by one or more body portions, in particular by one or more body portions of the patient's face. Examples for anatomical features are but are not limited to: a body axis of the patient, in particular a body axis of the patient's head, such as an interpupillary axis and/or a longitudinal axis of the patient's head. A further example for a body axis is a longitudinal axis of the patient's body. The body longitudinal axis of the patient's body may be defined by the patient's trunk, legs and/or head. Examples for landmarks are but are not limited to: a pupillary opening, a pupil center and a tip of the nose.

According to a further embodiment, the orientation analysis system comprises a detector system, which is configured to detect electromagnetic radiation emanating from a region that comprises one or more of the body portions and/or objects.

The radiation may emanate from the one or more body portions and/or objects in response to illuminating the one or more body portions and/or objects using illuminating radiation. The illuminating radiation may be generated using an illumination system of the ophthalmic laser system. The illumination system may include a light source which is configured to irradiate the one or more body portions and/or objects. The illumination system may also include an optical system, which is in the illumination beam path between the light source and the one or more body portions and/or objects. The illumination light source may simultaneously illuminate the one or more body portions and/or objects or may include a scanner for scanning an illumination light beam across the one or more body portions and/or objects.

By way of example, the emanating electromagnetic radiation may be scattered and/or reflected illuminating radiation. It is also conceivable that the emanating radiation includes fluorescent and/or phosphorescent radiation, which is excited by the illuminating radiation.

Alternatively, the emanating radiation may be radiation, which is generated by the body portions and/or objects, without being illuminated by an illumination system.

The illumination system may be configured so that the illumination of the one or more body portions and/or objects is homogeneous or substantially homogeneous. Alternatively, the illumination system may be configured to generate structured light illumination of the one or more body portions and/or objects. The structured light illumination may be generated using light having wavelengths in the visible wavelength range, in particular wavelengths between 380 nanometers and 780 nanometers. Additionally or alternatively, the structured light illumination may be generated using light having wavelengths in the infrared wavelength range, in particular between 780 nanometers and 1,400 nanometers. By way of example, the structured light illumination may be generated using one or more masks. Alternatively, the orientation analysis system may include a scanner for scanning a laser beam or collimated incoherent light across the one or more body portions and/or objects. The structured light illumination may be generated by the scanned laser beam or by the scanned collimated incoherent light. The structured light illumination may be configured so that an image acquired from the one or more body portions and/or objects using an imaging optical system of the orientation analysis system allows reconstruction of a surface topography of the body portions and/or objects.

According to a further embodiment, the detector system is configured to detect the electromagnetic radiation in a spatially resolved manner. By way of example, the orientation analysis system includes an image sensor, which is sensitive to the electromagnetic radiation. Additionally or alternatively, the orientation analysis system may include a scanner for successively receiving electromagnetic radiation from different portions of the object field and for guiding the received electromagnetic radiation to a detector.

According to an embodiment, the electromagnetic radiation has frequencies, which are within a range of between 10 GHz and 800 THz and/or has wavelengths, which are within a range of between 10 micrometers and 10 millimeters, in particular within a range of between 100 micrometers and 1 millimeter.

According to a further embodiment, the orientation analysis system is configured to acquire the orientation-related data using a magnetostatic and/or electrostatic field extending between a portion of the ophthalmic laser system, in particular between the movable portion of the beam delivery system and the one or more objects.

The orientation analysis system may include one or more magnets and one or more magnetic field sensors. The one or more magnets may be or may form part of the objects which are at a fixed position and/or orientation relative to the body portions and the magnetic field sensors may be part of the ophthalmic laser system. Alternatively, the one or more magnetic field sensors may be or may form part of the one or more objects and the magnets are part of the ophthalmic laser system.

Examples for objects which include the magnets or magnetic field sensors are but are not limited to: an eye fixation device, such as a suction ring and an object, which can be placed on the untreated eye of the patient or can be attached to the surgeon's finger, such as a ring.

Additionally or alternatively, the orientation analysis system may include one or more coils, such as Helmholtz coils, which are configured to generate a magnetic field at a location where one or more sensors of the orientation analysis system are located. The sensors may represent the objects or may form part of the objects, which have a fixed orientation and/or position relative to a respective body potion.

By way of example, the orientation analysis system may include a at least two Helmholtz coils, each of which being configured to generate a unidirectional magnetic field at the location of the sensors. The magnetic field sensors may include one or more Hall-effect sensors. By way of example, the orientation analysis system may include three pairs of Helmholtz coils. The coils of each pair may be arranged coaxial about a spatial axis and the axes are non-parallel relative to each other. The orientation analysis system further comprises an object, which has three Hall-effect sensors, for measuring components of the magnetostatic field generated by Helmholtz coils along three non-parallel axes.

According to a further embodiment, the orientation analysis system may be configured to detect capacitance between one or more electrodes, which are provided at the movable portion of the beam delivery system and one or more electrodes, which are provided at one or more of the objects. By way of example, each of a plurality of electrodes may be provided at a respective object and and the orientation analysis system may be configured to detect, for each of the electrodes a respective distance to one of a plurality of further electrodes, which are provided at the movable portion of the beam delivery system. The controller may be configured to determine, based on the detected distances, one or more parameters, which are (a) indicative of or (b) correlated with one or more, or all, parameters of the orientation of the patient's head around the axis of the eye to be treated.

According to a further embodiment, the orientation analysis system includes an imaging system for acquiring a two-dimensional image of the one or more body portions and/or objects. Additionally or alternatively, the orientation analysis system includes a surface topography acquisition system. Additionally or alternatively, the orientation analysis system includes 3D imaging system.

The surface topography acquisition system may include a LIDAR scanning system for acquiring a surface topography of the one or more body portions and/or objects. LIDAR is an acronym of “light detection and ranging”. The LIDAR scanning system may include a laser, which forms spot-shaped cross-sectional laser beam and a scanner. The laser may be configured to generate a pulsed laser beam to measure the time of flight (ToF LIDAR), an amplitude-modulated continuous wave laser beam (AMCW LIDAR) or a freqency-modulated continuous wave laser beam (FMCW LIDAR). The scanner may be configured to deflect the laser beam in two dimensions. The scanner may be configured to scan a surface, which includes surfaces of the one or more body portions and/or objects.

Additionally or alternatively, the surface topography analysis system may include a 3D time of flight camera system. The 3D time of flight camera system may include a light source and a 3D image sensor. The light source may be configured as a laser and/or a LED. The light source may be a non-scanning light source. The light emitted by the light source may be pulsed, amplitude modulated or may be a stroboscopic light source generating a square wave.

Additionally or alternatively, the surface topography acquisition system may include at least one RF (radio frequency) detector and at least one RF tag. The RF tag may be an object or may be attached to an object. The RF detector may include one or more antennas for detecting signals emitted from the RF tag. Based on the relative strengths and/or phase differences of the RF signals emitted from RF tag and detected by the RF detector, the controller may determine one or more parameters of a position and/or an orientation of the RF tag relative to the RF detector.

Additionally or alternatively, the surface topography acquisition system may include an optical coherence tomography system and/or a confocal microscope.

The 3D imaging system may be configured as a stereoscopic 3D camera.

According to a further embodiment, the ophthalmic laser system includes one or more inclinometers and/or accelerometers. The controller may be configured to reduce or eliminate a degradation of the orientation-related data which is caused by a movement of the movable portion of the beam delivery system relative to the patient's head.

The degradation of the orientation-related data may be caused by a supporting arm, which displaceably supports the movable portion of the beam delivery system and/or by one or more guides, which support the movable portion of the beam delivery system. Movements of the supporting arm may be caused by external forces, such as providing user input on a tough screen using fingers.

According to an embodiment, an optical path of the orientation analysis system traverses one or more optical elements of the beam delivery system in particular one or more optical elements of the movable portion of the beam delivery system. Additionally or alternatively, the optical path traverses an optical element, in particular a patient interface, which is arranged in a beam path of the treatment laser beam between the beam delivery system and the eye to be treated.

The optical path may be an imaging beam path of an imaging system, which acquires a frontal image of the eye to be treated, in particular an image of a region surrounding the optical axis of the beam delivery system at a location within the anterior segment of the eye. By way of example, the imaged region may include the pupil and/or at least a portion of the the iris of the eye to be treated. The imaging beam path may extend through one or more optical elements of the patient interface, in particular through a contact element of the patient interface, which contacts an anterior surface of the cornea.

Additionally or alternatively, the optical path of orientation analysis system, which extends through one or more optical elements of the movable portion of the delivery system and/or the optical element of the patient interface may be an optical path of the measuring arm of an optical coherence and/or a confocal microscope. The optical coherence system and/or confocal microscope may be configured to acquire one or more cross-sectional images of the cornea and/or of at least a portion of the natural lens of the eye. The image planes of the cross-sectional images may be non-parallel to each other. The ophthalmic laser system may be configured to allow the user and/or the controller to use the images of the non-parallel image planes to align the axis of the eye to be treated with the optical axis of the movable portion of the beam delivery system.

According to an embodiment, the orientation-related data includes a pictorial, symbolic and/or topographic representation and/or a 3D image, which comprises one or more of the body portions and/or objects. The ophthalmic laser system may include a display device, which is operatively coupled with the controller. The controller may be configured to control the display device present the pictorial, symbolic and/or topographic representation and/or the 3D image to the user. The pictorial representation may be an image.

According to an embodiment, the controller is configured to determine, automatically or using user input, a position and/or an orientation of the one or more body portions and/or objects in the representation and/or 3D image.

The ophthalmic laser system may include a user interface configured to receive user input for semi-automatically identifying one or more locations and/or image regions within the pictorial, symbolic and/or topographic representation and/or 3D image. By way of example, the graphical user interface may be configured to receive user input via pointing device, such as a computer mouse, wherein the user input identifies a the location and/or a boundary of the one or more regions.

The regions may correspond to the one body portions and/or objects.

According to an embodiment, the laser system is configured to display on a display device a graphical representation which is indicative of or correlates with a position and/or an orientation of the movable portion of the beam delivery system relative to a body portion of the patient, in particular relative to the patient's head.

The graphical representation may assist the surgeon in manually or semi-automatically (i.e. using the controller and user input) position the movable portion of the beam delivery system relative to the patient's head. Thereby, undesirable collisions between the patient's head and the movable portion of the beam delivery system can be avoided.

According to an embodiment, the laser system is configured to determine, based on the orientation-related data, a one or more parameters of a positioning movement of the movable portion of the beam delivery system. The determined one or more parameters may include one or more parameters for adjusting the orientation of the movable portion of the beam delivery system relative to the patient's head about the axis of the eye to be treated. The one or more parameters may be indicative of one, two or three translational degrees of freedom of the movable portion. Additionally or alternatively, the one or more parameters may be indicative of one, two or three rotational degrees of freedom of the movable portion. The parameters may specify the movement relative to the base of the ophthalmic laser system. The parameters may relate to a path for moving the laser applicator between both eyes of the patient in order to perform laser treatments for each of the eyes. The controller of the ophthalmic laser system may be configured to determine parameters of the positioning movement which are indicative of six degrees of freedom based on surface topography data, which have been acquired using the orientation-analysis system.

According to an embodiment, at least one of the body portions include at least a portion of the patient's head, in particular (a) at least a portion of the untreated eye or (b) at least a portion of the eye to be treated.

According to an embodiment, at least one of the body portions comprises at least a portion of a surrounding anatomy of an eyeball of at least one of the patient's eyes, in particular a lacrimal punctum and/or a lacrimal caruncle.

According to an embodiment, the orientation analysis system comprises at least two imaging systems. A first one of the imaging systems may be configured to image (i) at least a portion of the eye to be treated; and/or (ii) at least a portion of an optical element, in particular a patient interface, which is in the beam path of the treatment laser beam between the beam delivery system and the eye of the patient. The second one of the imaging systems may be configured to image at least a portion of the untreated eye and/or at least a portion of an object, which is in contact with and/or which covers at least a portion of the untreated eye.

According to an embodiment, the orientation analysis system comprises an imaging system configured to image (i) at least a portion of the eye to be treated; and/or (ii) at least a portion of an optical element, in particular a patient interface, which is in the beam path of the treatment laser beam between the beam delivery system and the eye of the patient. The orientation analysis system may further comprise a system for acquiring a 3D image and/or a topographical representation of at least a portion of the untreated eye and/or an object, which is in contact with and/or which covers at least a portion of the untreated eye.

According to an embodiment, one of the objects is at least a portion of an eye fixation device, in particular at least a portion of a suction ring.

A position of a portion of the eye fixation device which contacts the cornea may be centered or substantially centered with the pupil of the eye. The eye fixation device may have a shape which is visually asymmetric with respect to an axis of the portion of the eye fixation device, which contacts the cornea. By way of example, the eye fixation device may include a handle, which is used by the surgeon to position the eye fixation device relative to the patient's eye. The handle may visually indicate to the surgeon an orientation of the eye fixation device about the axis of the eye. The handle may also limit the angular range of the orientation of the eye fixation device relative to the patient's head, when being attached to the eye to be treated. Due to the limited angular range, the orientation of the eye fixation device is indicative of or at least correlates with the orientation of the patient's head in a plane, which is perpendicular to the axis of the eye to be treated. Moreover, determining a surface topography of the eye fixation device using the orientation analysis system allows adjusting an inclination of the laser applicator relative to an inclination of the eye fixation device. Thereby, undesirable shear forces during the docking procedure can be avoided.

According to an embodiment, the controller is configured to determine, automatically or based on user input, a position and/or an orientation of the eye fixation device, in particular a position and/or an orientation of one or more marks, in particular infrared marks, of the eye fixation device based on the orientation-related data. The one or more infrared marks may be infrared absorbing or infrared reflecting marks.

The orientation analysis system may include an imaging system for imaging at least a portion of the eye fixation device onto an image sensor of the imaging system when the eye fixation device is attached to the patient's eye. An imaging beam path of the imaging system may traverse one or more optical elements of the beam delivery system, in particular of the movable portion of the beam delivery system.

According to an embodiment, the orientation analysis system includes an aiming system for aiming at one or more of the body portions and/or objects along an aiming direction.

The aiming system may be configured to aim at one or more body portions of the patient, such as at a foot of the patient, at the head of the patient, at one or both eyes of the patient, at the mouth of the patient or at a foot of the patient. The aiming system may be configured to simultaneously aim at two separate body portions (such as both eyes of the patient) or to align the aiming system with a longitudinal shape of a body portion (such as with the longitudinal shape of a mouth).

According to an embodiment, the aiming system includes a light source for generating an aiming beam extending along the aiming direction. Additionally or alternatively, the aiming system includes a mechanical indicator, which visibly indicates the aiming direction. Additionally or alternatively, the aiming system, comprises an array of light sources, which are arranged in a circumferential manner, wherein the aiming system is configured so that one or more active light sources of the array of light sources indicate the aiming direction.

According to an embodiment, the aiming system is configured so that the aiming direction is adjustable by the user and/or controller.

Embodiments of the present disclosure pertain to an ophthalmic laser system having a beam delivery system. The laser system is configured to perform laser treatments on an eye of a patient using a treatment laser beam. The laser system includes a beam delivery system for focusing the treatment laser beam so that the treatment laser beam travels toward the patient's eye. The laser system further includes a controller operatively coupled with the beam delivery system and configured to control the beam delivery system to position a focus of the laser beam at different locations within the eye or on an anterior surface of the cornea of the eye. The laser system further includes a guide configured to allow moving at least a portion of the beam delivery system to adjust an orientation of at least the movable portion to different orientations of the patient's head measured around an axis of the eye to be treated. The laser system further includes an orientation indicating system which is configured to indicate, to the user, a relative orientation between (a) an orientation of a body portion of the patient and/or an object, which is in contact with and/or covers at least a portion of the body portion; and (b) an orientation of the movable portion of the beam delivery system, wherein the relative orientation is relative to an axis of the eye to be treated.

The orientation indicating system may be configured to indicate whether or not the movable portion has an orientation around the axis of the eye and relative to the patient's head, which (i) corresponds or substantially corresponds to a pre-determined target orientation; or which (ii) is within or substantially within a pre-determined target range.

According to an embodiment, the orientation indicating system comprises a light source. A position and/or an orientation of a light emitting surface of the light source may be indicative of or correlated with the orientation of the movable portion of the beam delivery system. Additionally or alternatively, a direction of light emitted by the light source may be indicative of or correlated with the orientation of the movable portion of the beam delivery system. The direction of light may have a fixed orientation relative to the movable portion of the beam delivery system. The orientation may be measured in a plane perpendicular to the axis of the eye to be treated.

According to an embodiment, the light source is a laser, in particular a line laser.

According to a further embodiment, the orientation indicating system includes an aiming system for aiming at one or more pre-defined body portions of the patient.

According to a further embodiment, the orientation indicating system comprises a light source. The orientation indicating system may be configured so that light, which is emitted from the light source generates a visible mark on the patient's body of the patient or on an object, which is attached to and/or covers at least a portion of the patient's body.

According to a further embodiment, a position and/or an orientation of the visible mark is indicative of or correlates with the relative orientation.

According to a further embodiment, the orientation indicating system includes a mechanical direction indicator, wherein the indicated direction is indicative of or correlates with the orientation of the movable portion of the beam delivery system. The mechanical direction indicator may be a projection, which extends from the housing of the laser applicator. The mechanical direction indicator may be configured as a projection which may be in the form of a tab. However, other shapes of the mechanical direction indicator are conceivable. The projection may be in the form of a tab. The mechanical direction indicator may have a fixed orientation relative to the movable portion of the beam delivery system. The orientation may be measured in a plane perpendicular to the axis of the eye to be treated.

According to a further embodiment, the ophthalmic laser system includes a supporting arm, wherein at least the portion of the beam delivery system is displaceably supported by, or is part of a free end of the supporting arm.

According to a further embodiment, a second end of the supporting arm, which is opposite to the free end is connected to a base of the laser system, which supports the supporting arm. Additionally or alternatively, the second end includes an interface for connecting the supporting arm to a further component at the second end of the supporting arm.

According to a further embodiment, the laser system includes a laser source for generating the treatment laser beam. A second end of the supporting arm, which is opposite to the free end may connected to a base of the laser system, which supports the supporting arm. The base may house at least a portion of the laser source.

According to a further embodiment, the supporting arm comprises one or more sensors which are operatively coupled with the controller, and which comprise one or more of: (a) a linear and/or an angular position sensors; and/or (b) an inclinometers and/or an accelerometer. The controller may be configured to determine one or more, or all parameters of a position and/or orientation of the laser applicator based on an output of the one or more sensors. The determined position and/or orientation may be determined relative to the base of the ophthalmic laser system.

According to a further embodiment, the supporting arm comprises one or more linear and/or angular position sensors which are operatively coupled with the controller. The controller may be configured to determine one or more, or all parameters of a position and/or orientation of the laser applicator based on an output of the one or more linear and/or angular sensors.

Embodiments of the present disclosure pertain to a method of operating an ophthalmic laser system having a beam delivery system, for performing laser treatments of an eye of a patient using a treatment laser beam. The laser system includes a beam delivery system for focusing the treatment laser beam so that the treatment laser beam travels toward the eye to be treated. The laser system further includes a controller operatively coupled with the beam delivery system and configured to control the beam delivery system to position a focus of the laser beam at different locations within the eye or on an anterior surface of the cornea of the eye. The laser system further includes a guide configured to allow moving at least a portion of the beam delivery system to adjust an orientation of at least the movable portion to different orientations of the patient's head measured around an axis of the eye to be treated. The method includes acquiring, using an orientation analysis system, orientation-related data using one or more body portions of the patient and/or one or more objects, each of which being in a fixed position and/or orientation relative to a respective body portion so that the orientation-related data depend on an orientation of the one or more body portions around the axis of the eye. The method further includes moving, by a user and/or a controller of the laser system, using the orientation-related data, the movable portion of the beam delivery system so that during the laser treatment, the movable portion has an orientation around the axis of the eye and relative to the patient's head, which corresponds or substantially corresponds to a pre-determined target orientation or which is within or substantially within a pre-determined target range.

Embodiments of the present disclosure pertain to a method of operating an ophthalmic laser system having a beam delivery system, for performing laser treatments of an eye of a patient using a treatment laser beam. The laser system includes a beam delivery system for focusing the treatment laser beam so that the treatment laser beam travels toward the eye to be treated. The laser system further includes a controller operatively coupled with the beam delivery system and configured to control the beam delivery system to position a focus of the laser beam at different locations within the eye or on an anterior surface of the cornea of the eye. The method includes acquiring, using an orientation analysis system, orientation-related data using one or more body portions of the patient and/or one or more objects, each of which being in contact with and/or cover a respective body portion, so that the orientation-related data depend on an orientation of the one or more body portions around the axis of the eye. At least one of the body portions and/or objects is outside the eye to be treated. The controller is further configured to determine, based on the orientation-related data (i) one or more parameters of the laser positioning data; and/or (ii) whether an exit optical element of the beam delivery system is positioned in front of the left or in front of the right eye of the patient.

According to an embodiment, method further includes placing, using the user and/or the ophthalmic laser system, at least one of the objects so that the object is in contact with and/or covers a body portion of the patient's body. The method may further include acquiring the orientation-related data at least from a portion of the placed objects. The body portion may be a pre-defined body portion, in particular an anatomically and/or functionally pre-defined body portion.

According to an embodiment, the placing of the object comprises placing the object on a further object, in particular a portion of a tissue, which is in contact and/or which covers the body portion.

According to a further embodiment, the placed object is an anatomical portion of the surgeon, in particular a finger or a tip of the finger of the surgeon's hand.

Embodiments of the present disclosure pertain to an ophthalmic laser system for performing laser treatments of an eye using a treatment laser beam. The laser system comprises a laser applicator, which comprises an optical system through which the treatment laser beam exits the laser applicator in a direction toward the patient's eye. The laser system further comprises a beam delivery system for focusing the treatment laser beam so that the treatment laser beam travels toward the eye to be treated. The laser system further comprises a supporting arm. A free end of the supporting arm comprises or displaceably supports the laser applicator. The laser system further comprises a controller operatively coupled with the beam delivery system. The controller is configured to control the beam delivery system to position a focus of the laser beam at different locations within the eye or on an anterior surface of the cornea of the eye based on laser positioning data, which are indicative of or correlated with the locations within the eye to be treated. The laser system further includes a giga-terahertz imaging system for acquiring image data from (a) one or more body portions of the patient; and/or (b) one or more objects, each of which having a fixed position and/or orientation relative to a respective body portion. The giga-terahertz imaging system is configured to use electromagnetic radiation having frequencies in a range of between 10 GHz and 100 THz. The laser system is further configured to: (a) allow a user and/or the controller to adjust a relative position and/or orientation of the laser applicator relative to the patient's head using the image data; and/or (b) determine, using the controller, the laser positioning data based on the imaging data.

According to a further embodiment, the radiation source is configured to generate electromagnetic radiation having frequencies in a range of between 30 GHz and 50 THz.

According to a further embodiment, the terahertz and/or gigahertz imaging system includes a scanner for scanning a beam of the electromagnetic radiation across the one or more body portions and/or objects.

According to a further embodiment, the controller is configured to determine, based on the image data, an outer contour of at least a portion of the patient's body. The outer contour may be a contour of at least a portion of the patient's skull, chin, trunk and/or legs.

According to a further embodiment, the free end of the supporting arm includes or displaceably supports at least a portion of the giga-terahertz imaging system.

Embodiments of the present disclosure pertain to an ophthalmic laser system for performing laser treatments of an eye. The laser system includes a laser applicator, which comprises an optical system through which the treatment laser beam exits the laser applicator in a direction toward the patient's eye. The laser system further includes a supporting arm, wherein a free end of the supporting arm includes the laser applicator or displaceably supports the laser applicator. The laser system further includes a controller. The supporting arm comprises a controllable visual indicator, wherein the controller is in operative connection with the visual indicator to control generation of a visual signal. The visual indicator includes (a) a curved surface, from which signal light of the visual signal emanates, wherein the curved surface forms at least a circumferential portion of an outer circumferential surface of the supporting arm; and/or (b) a plurality of surfaces, wherein from each of the surfaces, signal light of the visual signal emanates, wherein the plurality of surfaces are distributed at least around a circumferential portion of the outer circumferential surface of the supporting arm.

The visual signal may be indicative of information. The information may be encoded in an intensity variation in a color variation and/or in a variation of a subset of the plurality of surfaces from which the visual signal emanates. According to a further embodiment, the supporting arm comprises at least one arm segment, wherein the visual indicator is arranged at a supporting structure of the supporting arm, which supports the arm segment. The supporting structure may include at least a portion of a rotational guide for pivoting the arm segment about a horizontal axis. The laser system may be configured so that the supporting structure is displaceably mounted on the base of the ophthalmic laser system. By way of example, the supporting structure may be connected to the base via one or more further arm segments. The supporting structure may be configured as a joint, which connects two consecutive arm segments. Alternatively, the supporting structure may be fixedly connected to the base. Additionally or alternatively, the laser system may be configured so that the supporting structure can be controllably translationally displaced relative to the base. By way of example, the ophthalmic laser system may include a positioning stage, which is mounted on the base. The supporting structure may be mounted on the positioning stage so that the mounting structure can be translationally displaced relative to the base at least in two horizontal dimensions.

The arm segment may be the most distal arm segment of all rotatable arm segments of the supporting arm. The supporting arm may be configured so that the arm segment is rotatable about a horizontal and/or about a vertical axis. The arm segment may form part of a multi bar linkage of the supporting arm. The multi bar linkage may be configured so that an orientation of a first end of the multi bar linkage relative to a second end of the multi bar linkage is maintained while moving the first end relative to the second end. The multi bar linkage may be a parallel linkage and/or a four bar linkage. The multi bar linkage may be balanced in order to provide at least partial gravity counterbalancing for the laser applicator. The multi bar linkage may be the only component of the supporting arm, which is rotatable about a horizontal axis and/or may be the most distal component of the supporting arm which is rotatable about a horizontal axis.

It should be appreciated that the present disclosure is not limited to visual indicators, which are arranged at a joint and/or at a supporting structure of the supporting arm. It is also conceivable that the visual indicator is mounted to or is part of an arm segment or a multi bar linkage of the supporting arm. The arm segment or the multi bar linkage may be the most distal arm segment or the most distal multi bar linkage of the supporting arm. The arm segment or multi bar linkage may be rotatable about a horizontal axis. Additionally, the arm segment or multi bar linkage may also be rotatable about a vertical axis. An orientation of the horizontal rotation axis within a horizontal plane may be adjustable by the supporting arm.

According to a further embodiment, the supporting arm comprises two arm segments, wherein the two arm segments are connected via one or more joints. The visual indicator may be arranged at the one or more joints.

According to a further embodiment, the outer circumferential surface has a horizontal or substantially horizontal circumference. A circumferential direction of the circumferential surface may be oriented or substantially oriented horizontally. In other words, an axis of the circumferential surface may be oriented vertically or substantially vertically.

The circumferential surface may have a cylindrically outer circumferential shape. However, other outer shapes of the circumferential surface are also conceivable, such as an oval, rectangular, rhomboidal, polygonal outer shape. The polygonal outer shape may have three or more straight edges.

According to a further embodiment, a first arm segment of the two arm segments is rotatable about a vertical or substantially vertical axis and the second arm segment of the two arm segments is rotatable about a horizontal or substantially horizontal axis.

According to a further embodiment, the second arm segment is also rotatable about a vertical axis in order to adjust an orientation of the second arm segment relative to the first arm segment.

According to a further embodiment, the first arm segment is connected to the laser applicator via the second arm segment.

According to a further embodiment, the controller is configured so that the visual signal indicates a stage of the laser treatment. The controller may be configured to indicate, using the visual indicator, the stage of the laser treatment in a pre-defined time correlation, in particular in time synchronization with a progress of the ophthalmic laser system.

According to a further embodiment, the controller is configured so that the visual signal indicates an operational state of a laser treatment performed using the ophthalmic laser system. The controller may be configured to indicate, using the visual indicator, the operational state of the laser treatment in a pre-defined time correlation, in particular in time synchronization with a progress of the ophthalmic laser system.

According to a further embodiment, the controller and the visual indicator are configured so that the visual indicator indicates a warning message to a user.

According to a further embodiment, the supporting arm is configured so that the laser applicator is positionable relative to the base in three dimensions.

According to an embodiment, the visual indicator inlcludes one or more light emitting diodes (LED). The visual indicator may include a plurality of LEDs which are arranged circumferentially distributed around at least the circumferential portion.

Additionally or alternatively, the visual indicator may include a light-diffusing optical fiber. A fiber axis of the light-diffusing optical fiber may be arranged at least partially around the circumferential surface to form one or more windings.

According to an embodiment, the visual indicator comprises one or more emitting diodes (LED). The plurality of LEDs may be arranged on a carrier structure. The carrier structure may be in the form of or substantially in the form of a board. At least a portion of the carrier structure may be configured as a circuit board. The board may be in a plane or substantially plane shape. Alternatively, the board may be curved, wherein the LEDs are mounted on an outer circumferential surface of the board. The carrier structure may be in the form substantially in the form of at least a circumferential portion of a ring.

According to a further embodiment, the visual indicator comprises a plurality of light sources, wherein each of the light sources includes or is covered by one of the surfaces from which signal light emanates and which are distributed around at least the circumferential portion.

According to an embodiment, the visual indicator includes a light reflector which is illuminated by one or more light sources of the visual indicator. The reflector may be configured for specular or diffuse reflection of signal light, which is emitted from the one or more light sources of the visual indicator. The reflector and/or the light diffusing sheet may be in the beam path of the signal light between the one or more light sources and a cover of the light indicator, which covers the reflector. The cover may also cover the one or more light sources of the visual indicator. At least a portion of the cover may be made from a light diffusing material and/or may be transparent for one or more wavelengths, which are emitted from one or more light sources of the visual indicator.

Embodiments of the present invention pertain to an ophthalmic laser system for performing laser treatments of an eye. The laser system includes a base, which houses at least a portion of a laser source of the laser system wherein the laser source is configured for generating a treatment laser beam for performing the laser treatments. The laser system further includes a laser applicator, which comprises an optical system through which the treatment laser beam exits the laser applicator in a direction toward the patient's eye. The laser system further includes a supporting arm and a controller. The supporting arm is connected to the laser applicator at a first end of the supporting arm. A second end of the supporting arm may be connected to the base, and, additionally or alternatively, the supporting arm comprises an interface for connecting the supporting arm to a further component at a second end of the supporting arm. The supporting arm may be configured so that the laser applicator is positionable relative to the base in three dimensions. The laser applicator may be positionable while maintaining a vertical orientation of the laser applicator. The laser system may further comprise a motorized three-axis positioning system, which is operatively coupled to the controller for positioning the laser applicator relative to at least a portion of the supporting arm or relative to the entire supporting arm. The positioning system may be configured so that the laser applicator is positionable in three dimensions.

The laser system may include an articulated beam guide tube. At least a portion of the articulated beam guide tube may extend between a first location where the treatment laser beam exits from the base or from the supporting arm and a second location, where the treatment laser beam enters into the supporting arm or into the laser applicator. The laser system may include a coupling arrangement for coupling the articulated beam guiding tube to the supporting arm at one or more locations along the beam guiding tube between the first location and the second location.

The supporting arm may be connected to the laser applicator at a first end. The second end of the supporting arm may be connected to the base and/or may be connectable to a further component at the second end of the supporting arm. The further component may be external to the ophthalmic laser system. By way of example, the further component may be a second base or may be a wall, a ceiling or a floor of a building.

The base of the laser system may be configured as a moveable base, which is moveable across the floor surface. The mobile base may include a plurality wheels, such as four wheels for moving the base across the floor surface. The mobile base may be configured so that the entire laser surgical system can be moved by one person across the floor. The supporting arm and the laser applicator may only be supported by the base.

The laser source may be an infrared laser source. The infrared laser source may be configured as a femtosecond laser source. A pulse energy of the laser pulses may be greater than 1 nanojoule, or greater than 10 nanojoule or greater than 50 nanojoule. The pulse energy may be less than 20 microjoule, or less than 15 microjoule or less than 10 microjoule. A pulse duration of the pulsed laser beam may be less than 800 femtoseconds, or less than 500 femtoseconds, or less than 300 femtoseconds, or less than 150 femtoseconds, or less than 100 femtoseconds. The pulse duration may be greater than 10 femtoseconds or greater than 50 femtoseconds. A repetition rate of the pulsed laser beam may be greater than 50 kHz or greater than 80 kHz. The repetition rate of the pulsed laser beam may be less than 10 MHz or less than 1 MHz. A center wavelength of the pulsed laser beam, which is incident on the eye may be in a range of between 800 nanometers and 1400 nanometers, or between, 900 nanometers and 1400 nanometers, or between 1000 nanometers and 1100 nanometers, or between 1010 nanometers and 1050 nanometers.

The infrared laser source may be configured so that the laser pulses have a pulse energy so that the laser beam generates photodisruption within corneal tissue or within the natural lens of the patient's eye. The photodisruption may be caused by laser-induced optical breakdown. Alternatively, a pulse energy of the laser pulses may be below a threshold for generating laser-induced optical breakdown. By way of example, a plurality of pulses, which have a pulse energy below the threshold for generating laser-induced optical breakdown may be overlapped in a manner so as to generate tissue separation within the cornea.

The infrared laser source may include a precompensator for at least partially pre-compensating a change of the group delay dispersion (GDD) of the laser pulses, which is induced by components of the laser optical system, which are in the beam path of the laser beam downstream of the laser source. If a laser pulse has a positive GDD, longer wavelengths of the laser pulse propagate faster than shorter wavelengths. A positive group delay dispersion therefore corresponds to a material dispersion, which is typical in transparent media, since red wavelengths experience a lower refractive index compared to blue wavelengths. The pre-compensator may be configured to reduce the group delay dispersion. By way of example, the reduced group delay dispersion generated by the pre-compensator may have a lower positive or a more negative group delay dispersion.

A lateral diameter of the focus of the treatment laser beam of the infrared laser source within the cornea or the lens may be smaller than 10 micrometers, or smaller than 6 micrometers. The diameter may be greater than 3 micrometers. The lateral diameter may be measured in a direction perpendicular to an optical axis of the laser optical system. The lateral diameter may be measured as an 80% encircled energy diameter.

Alternatively, the laser source may be configured as an excimer laser source. The laser system may be configured to generate a laser beam, which is focused on the anterior surface of the eye. A wavelength of the treatment laser beam generated by the excimer laser source may be greater than 150 nm or greater than 190 nm. The wavelength may be less than 400 nm or less than 200 nm. A pulse duration of the treatment laser beam generated by the excimer laser source may be shorter than 100 ns or shorter than 50 ns. The pulse duration may be greater than 1 ns or greater than 3 ns.

The controller may include a data processing system. The data processing system may include a computer system having a processor and a memory for storing instructions processable by the processor. The processor may execute an operating system. The data analysis system may further include a user interface configured to allow a user to receive data from the data processing system and/or to provide data to the data processing system. The user interface may include a graphical user interface.

The controller may be configured to determine a scanning path of the pulsed laser beam for scanning the laser focus on or within the cornea or within the natural lens of the patient's eye. The controller may be configured to determine the scanning path based on patient specific data.

The controller may be configured to generate the scanning pattern so that the laser pulses are overlapping or non-overlapping. A lateral displacement of neighboring laser pulses may be less than 30 micrometers, or less than 20 micrometers, or less than 10 micrometers. The displacement may be greater than 0.5 micrometers or greater than 1 micrometer.

The laser source may include an oscillator laser, which is configured to generate a train of low energy ultrashort pulses. A pulse energy of the low energy ultrashort pulses may be lower than 100 nJ or lower than 20 nJ, or lower than 10 nJ. The pulse energy may be greater than 1 pJ or greater than 100 pJ. The laser source may also include an amplifier, such as a regenerative amplifier or fiber amplifier for amplifying at least a portion of the low energy ultrashort pulses.

The base of the laser system may house a portion of the laser source. By way of example, the oscillator laser may be arranged in the base and the amplifier and/or a precompensator of the laser source, which is configured to reduce a group delay dispersion of the treatment laser beam may be arranged in the laser applicator. Alternatively, the base may house the entire laser source, such as the oscillator laser, the amplifier and the precompensator.

The base may include one or more housings. Each of the housings may house a portion of the laser source. Bay way of example, a first housing of the base may house a seed laser of the laser source and a second housing of the base may house an amplifier of the laser source, in particular a regenerative amplifier or a fiber amplifier.

The supporting arm may include one or more arm segments. The supporting arm may include a plurality of arm segments, which are connected in series. Neighboring arm segments of the supporting arm may be connected via one or more joints. An arm segment may be defined as a member of the supporting arm, which provides a non-articulated connection between a first end of the arm segment and a second end of the arm segment. The arm segment may provide a rigid or telescoping coupling between the first end of the arm segment and the second end of the arm segment. At one or at both ends of the arm segment, the arm segment may be attached or integrally connected to a joint. The term “integrally connected” is meant to indicate that a first element/feature extends or transitions in a continuous manner from a second element/feature and not as two separate and distinguishable elements.

The supporting arm may include at least one arm segment, which forms part of a multi bar linkage of the supporting arm. The multi bar linkage may be configured so that an orientation of a first end of the multi bar linkage relative to a second end of the multi bar linkage is maintained while moving the first end relative to the second end. The multi bar linkage may be a parallel linkage and/or a four bar linkage. The multi bar linkage may be balanced in order to provide at least partial gravity counterbalancing for the laser applicator.

The arm segments may be connected in series via rotational joints. Each of the rotational joints may be configured so that an orientation of neighboring arm segments relative to each other is adjustable. Each of the rotational joint may have either one or two rotation axes. The supporting arm may include at least one multi-axis joint, which has two or more rotation axes. Each of the rotation axes may be oriented substantially orthogonal or orthogonal relative to each other. One of the two rotation axes may be substantially oriented or oriented along a vertical direction.

The supporting arm may be configured so that a distance measured along a horizontal between the laser applicator and a location where the supporting arm is connected to the base may be adjustable. A maximum horizontal distance between the base and the applicator head may be greater than 50 centimeters or greater than 10 centimeters. The distance may be smaller than 5 meters or smaller than 3 meters.

The supporting arm may further be configured so that the laser applicator is movable in a circular or substantially circular arc path of travel. The arc may be in a horizontal or substantially horizontal plane. A radius of the arc may be greater than 10 centimeters or greater than 50 centimeters. The radius may be smaller than 5 meters or smaller than 3 meters. The radius may be defined by a longitudinal extent of an arm segment of the supporting arm, wherein the supporting arm is configured so that the arm segment can be rotated about a vertical or substantially vertical axis. Additionally or alternatively the supporting arm may be configured so that the arm segment can be rotated about a horizontal or substantially horizontal rotation axis. The arm segment may be part of a multi bar linkage, in particular part of a four bar or parallel linkage. Additionally or alternatively, the arm segment may be balanced for providing at least partial gravity counterbalancing for the laser applicator.

The positioning system may include, for each of the three axes, a guide, in particular a linear guide. The axes of the positioning system may be mutually perpendicular to each other. The positioning system may be configured as an XYZ positioning system, wherein the Z-axis is a vertical or substantially vertical axis. For each of the guides, the respective guide may be configured as a sliding guide and/or a roller guide. Each of the guide may include two mating guide members. The first guide member may be configured as a rail, may form a guide track and/or may define a guiding path. The second guide member may be configured as a carriage and/or may be configured to be movable along the guiding path and/or the guide track. The carriage may be a slide carriage and/or a roller carriage.

For each of the three axes of the positioning system, the range of movement may be less than 500 millimeters or less than 150 millimeters. The range of movement may be at least 1 millimeter or at least 3 millimeters.

For each of the three axes of the position mechanism, a positioning accuracy may be worse than 1 micrometer or worse than 10 micrometers. The positioning accuracy may be better than 500 micrometers or better than 100 micrometers.

For one or more or each of the axes, a positioning velocity may be user adjustable in a continuous or stepwise manner. The laser system may be configured to receive user input for adjusting a positioning velocity of one or more or each of the axes of the positioning system.

The positioning system may be arranged between the supporting arm and the laser applicator. However, it is also conceivable that the positioning system is part of the supporting arm.

According to an embodiment, the laser applicator comprises a manually operable control unit which is operatively coupled to the controller for performing the positioning of the laser applicator relative to at least the portion of the supporting arm based on user input received via the control element. The manually operated control unit may be configured for directional control, in particular for directional control in three dimensions. By way of example, the manually operated control unit may include a joystick and/or nay include one or more buttons. Each of the button may correspond to a direction of travel.

According to a further embodiment, the laser applicator includes an imaging system for acquiring a frontal image of at least a portion of the patient's eye at least during part of the positioning of the laser applicator relative to at least a portion of the supporting arm.

The controller of the laser system may include an image processing algorithm for determining, whether at least a portion of the frontal image is in focus. Additionally or alternatively, the image processing algorithm may be configured to determine one or more parameters, which depend on, or which are indicative of, a level of focus of at least a portion of the image. The image processing algorithm may include a segmentation algorithm for segmenting the frontal image. The image processing algorithm may determine one or more parameters, which depend on, or which are indicative of, a level of focus of one or more of the segmented image regions.

The imaging system may include an image sensor. The image sensor may include a two-dimensional ordered or unordered array of pixels. The image sensor may be sensitive to one or more wavelengths within a range of between 380 nanometers and 950 nanometers or within a range of between 380 nanometers and 1400 nanometers. The imaging system may include an imaging optical system for imaging a tissue portion which is arranged in an object plane of the imaging optical system onto the image sensor. At least a portion of the imaging optical system may be provided by a portion of the focusing optical system for focusing the treatment laser beam into the eye of the patient.

According to a further embodiment, the laser applicator comprises a beam combiner for combining an imaging beam path of the imaging system with a beam path of the treatment laser beam.

The beam combiner may be configured to deflect the treatment laser beam in a direction toward or substantially toward the eye. The beam combiner may include a mirror and/or a prism. The beam combiner may be configured as a dichroic beam combiner. The beam combiner may be in the beam path of the laser beam downstream of the focusing optical system, within the focusing optical system or upstream of the focusing optical system.

A distance of an object plane of the imaging system from the laser applicator may be configured, or may be adjustable so that, the distance substantially corresponds to a distance of the laser applicator from the cornea of the patient's eye during the laser treatment.

According to an embodiment, the laser applicator comprises a display device for displaying the frontal image at least during part of the positioning of the laser applicator relative to the supporting arm. The display device may mounted on or may be integrated within a housing of the laser applicator. The display may be visible to a user during operation of the manually operable control element.

According to a further embodiment, the laser applicator comprises an interaction measuring unit for generating an output signal which depends on at least a parameter of a mechanical interaction between the patient's eye and the laser applicator.

The interaction measuring unit may include a plurality of interaction measuring sensors. The sensors may be circumferentially distributed about an optical axis of laser applicator. The sensors may be circumferentially distributed at equal angles about the optical axis of the laser applicator. Additionally or alternatively, the sensors may be arranged at a same or substantially same radial distance from the optical axis of the laser applicator.

The measured parameter of the mechanical interaction may depend on a force or a directional component of a force, or may be a force or a directional component of a force. The force may occurs between the patient's eye and the laser applicator. Additionally or alternatively, the measured mechanical interaction may depend on a temporal change of the force or is the temporal change of the force between the patient's eye and the laser applicator.

The interaction measuring unit may include a plurality of interaction measuring sensors. One or more of the interaction measuring sensors may include a force sensor, a piezoelectric sensor and/or a strain gauge.

The interaction measuring unit may be configured to measure a magnitude of a projection of the force vector onto the optical axis of the laser applicator (i.e. the section of the optical axis at a location where the treatment laser beam exits from the laser applicator towards the patient's eye) and/or a magnitude of a projection of the force vector onto a plane perpendicular to the optical axis.

The strain gauge may be configured to measure a strain in a sensing material caused by a force. The strain may be compressive or tensile. The strain gauge may be configured as a foil gauge, a semiconductor gauge (which uses the effect of piezoresistivity), or a capacitive strain gauge. The piezoelectric sensor may use the piezoelectric effect in a piezoelectric material, such as quartz. The piezoelectric sensor may measure a compressive force, a tensile force and/or a shear force acting on the piezoelectric sensor. It is also conceivable that the force sensor includes an interferometric strain sensor or measures a force acting on a birefringent material. It is also conceivable that the force sensor is a fiber optic force sensor.

The force sensor may be arranged in a force path between the patient's eye and an optical system which focuses the treatment laser beam within the patient's eye.

According to a further embodiment, the laser applicator comprises a display device. The controller may be configured to generate data representative of graphical and/or textual information using the output signal generated by the interaction measuring unit. Additionally or alternatively, the controller may be configured to display the graphical and/or textual information on the display device at least during part of the positioning of the laser applicator relative to at least the portion of the supporting arm.

The graphical and/or textual information, which is generated using output signals of the interaction measuring unit may depend on the magnitude and direction of the force between the laser applicator and the eye, which may be determined using the interaction measuring unit.

Additionally or alternatively, the graphical and/or textual information may depend on a rate at which the magnitude and/or direction of the measured force changes.

According to an embodiment, the coupling arrangement comprises a tensile force transmitting connection. The tensile force transmitting connection may comprise a tension spring for transmitting the tensile force.

According to a further embodiment, the coupling arrangement comprises a guide. The guide may be configured as a lateral guide. The lateral guide may be configured to limit a lateral movement of a coupling member of the coupling arrangment relative to a longitudinal axis of an arm segment of the supporting arm. The coupling member may be rigidly attached or integrally connected to the articulated beam guide tube.

The lateral guide may guide a movement of the coupling member in a direction parallel or substantially parallel to a longitudinal axis of an arm segment of the supporting arm. The guide may be configured to limit, during a movement of the laser applicator, a variation of a vertical orientation of a plane defined by consecutive arm segments of the articulated beam guide tube.

According to a further embodiment, the laser applicator includes an optical coherence tomography (OCT) system which is configured for acquiring a cross-sectional image of at least a portion of the eye.

A center wavelength of the OCT measuring arm may be within a range of between 750 and 1400 nanometers. The optical coherence tomography system may be configured to acquire a cross-sectional image of the cornea and/or at least a portion of the natural lens of the eye. The OCT system may include a scanner. A scanner of the OCT system may be separate from the scanner of the optical system for scanning the treatment laser beam.

According to a further embodiment, the laser applicator comprises a beam combiner for combining a beam path of a measuring arm of the OCT system with a beam path of the treatment laser beam.

The beam combiner for combining the beam path of the treatment laser beam with the beam path of the measuring arm of the OCT system may be configured as a dichroic beam combiner. At least part of the beam combiner for combining the beam path of the measuring arm of the OCT system with the treatment laser beam may be provided by at least a portion of the beam combiner for combining the imaging beam path of the imaging system with the beam path of the treatment laser beam.

According to an embodiment, the supporting am includes an arm segment which is rotatable about a horizontal or substantially horizontal axis. According to a further embodiment, the arm segment is part of a multi bar linkage, in particular a four bar linkage, such as a parallel linkage. The multi bar linkage may be configured so that during rotation of the arm segment about the horizontal axis, the vertical orientation of the laser applicator is maintained.

According to a further embodiment, the supporting arm comprises a first arm segment and a second arm segment which are connected to each other in series via an intermediate joint. The first arm segment may be rotatable about a vertical or substantially vertical axis and the second arm segment is rotatable about a horizontal or substantially horizontal axis.

According to a further embodiment, the intermediate joint system is configured to allow the second arm segment to rotate (a) about the horizontal or substantially horizontal axis and (b) about a vertical or substantially vertical axis. The intermediate joint system may include two joints, which are rigidly attached or integrally connected to each other. A first one of the two joints has a vertically or substantially vertically oriented rotation axis and the second one of the two joints has a horizontally or substantially horizontally oriented rotation axis. The first joint may be attached or integrally connected to the first arm segment and the second joint may may be attached or integrally connected to the second arm segment.

According to a further embodiment, the second arm segment is part of a multi bar linkage, in particular a four bar linkage such as a parallel linkage.

According to a further embodiment, the supporting arm comprises a counterbalancing mechanism to provide at least partial gravity counterbalancing for the applicator head.

According to a further embodiment, the counterbalancing mechanism include one or more springs. Each of the springs may be configured as a gas spring or as a mechanical spring.

According to a further embodiment, the supporting arm comprises a braking and/or locking system for arresting a movement of the second end of the supporting arm relative to the first end of the supporting arm. The braking and/or locking system may include one or more lockable joints and/or brakes for braking a movement of portions of the joints relative to each other. The locking mechanism of the lockable joint may be based on a positive locking of members of the joint, which are otherwise movable relative to each other.

According to a further embodiment, the laser applicator comprises a manually operable control unit for selectively activating and deactivating the braking and/or locking system based on user input received via the control unit.

According to a further embodiment, the laser system comprises an interaction measuring unit configured for generating an output signal which depends on a mechanical interaction between the patient's eye and the laser applicator. The controller may be operatively connected to the interaction measuring unit and the braking and/or locking system. The controller is configured to receive the output signal generated by the interaction measuring unit and to determine based on the received output signal, whether or not to deactivate the braking and/or locking system.

The articulated beam guiding tube may include one or more joints, each of which connecting neighboring longitudinal tube segments. Each of the tube elements may be define a linear laser beam path, which extends along the longitudinal axis of the tube segment. For each of the tube segments, the respective tube segment may either be rigid or maybe extendable along a longitudinal axis of the respective tube element.

Each of the joints of the articulated beam guiding tube may include a mirror system. The mirror system may include one or more mirrors. The mirror system may be configured to deflect the treatment laser beam which is emitted from a first one of the neighboring tube elements which are connected to the joint into the second one of the neighboring tube elements.

According to a further embodiment, the supporting arm or the laser applicator has a rotational joint, which has a vertically extending rotation axis for rotating the laser applicator about a vertical axis and relative to at least a portion of the supporting arm.

According to a further embodiment, the laser system comprises a locking system which is configured for locking the rotational joint having the vertically extending rotation axis.

According to a further embodiment, the laser applicator includes a focusing optical system for focusing the treatment laser beam within the eye and/or an axial scanning system for scanning the laser focus along an axis of the laser beam; and/or a beam deflection scanning system for scanning the laser beam through deflection of the laser beam.

Further embodiments of the present disclosure pertain to a method of positioning a laser applicator of an ophthalmic laser system relative to a patient's eye. The method comprises positioning a laser applicator relative to the patient's eye using a supporting arm. The supporting arm may be connected to the laser applicator at a first end of the supporting arm and. A second end of the supporting arm (a) may be connected to the base and/or (b) may comprise an interface for connecting the supporting arm to a further component at the second end of the supporting arm. The laser system may be configured for generating a treatment laser beam for performing the laser treatments. The supporting arm may configured so that the laser applicator is positionable relative to the base base while maintaining a vertical orientation of the laser applicator. The method may further comprise positioning the laser applicator relative to the supporting arm using a motorized three-axis positioning system;

According to a further embodiment, the method includes acquiring a frontal image of the eye using an imaging system of the laser applicator. The method may also include displaying, during at least part of the positioning of the laser applicator relative to the supporting arm, the frontal image on a display device of the laser system. The displayed frontal image may be a real-time image.

According to a further embodiment, during the part of the positioning, a distance of the focal plane from the laser applicator substantially corresponds to a pre-defined distance of the laser applicator from the patient's eye, in particular from a cornea of the patient's eye.

According to a further embodiment, the method further comprises generating, by an interaction measuring unit, an output signal, which depends on a mechanical interaction between the patient's eye and the laser applicator. The method may further include determining, using a controller of the laser system, textual and/or graphical information based on the output signal. The method may further include displaying, at least during part of the positioning of the laser applicator, the textual and/or graphical information.

A time span during which the frontal image is displayed and a time span during which the textual and/or graphical information based on the output signals of the interaction unit may be identical, overlapping or non-overlapping.

According to a further embodiment, the method includes generating, by an interaction measuring unit, an output signal, which depends on a mechanical interaction between the patient's eye and the laser applicator. The method may also include determining, using a controller of the laser system and based on the output signal, whether or not to deactivate brakes of the supporting arm, which arrest a movement of the second end of the supporting arm relative to the first end of the supporting arm.

### BRIEF DESCRIPTION OF THE DRAWINGS

FIG. 1A is a perspective view of a laser system according to an exemplary embodiment.

FIG. 1B is a schematic cross-sectional illustration of a lower portion of the laser applicator and a contact element according to a first and second variant of the laser system according to the exemplary embodiment;

FIG. 1C is a schematic illustration of a window of a graphical user interface of a first and second variant of the laser system according to the exemplary embodiment;

FIG. 1D is a schematic illustration of a sterile drape used for performing laser treatments using the laser system according to the exemplary embodiment;

FIGS. 1E and 1F are further schematic illustrations of the window of the graphical user interface of the first and second variants of the laser system according to the exemplary embodiment;

FIG. 1G is a schematic cross-sectional illustration of a lower portion of the laser applicator and a contact element of a third variant of the laser system according to the exemplary embodiment;

FIG. 1H is a schematic illustration of anatomical landmarks and anatomical features which are used by the third variant of the laser system according to the exemplary embodiment in order to determine orientation-related data;

FIG. 1I is a schematic illustration of mounting locations of the orientation analysis system of the third variant of the laser system according to the exemplary embodiment:

FIG. 1J is a schematic cross-sectional illustration of a lower portion of the laser applicator and a contact element of a fourth variant of the laser system according to the exemplary embodiment;

FIGS. 1K and 1L are schematic illustrations of the orientation analysis system of a fifth variant of the laser system according to the exemplary embodiment;

FIG. 1M is a schematic illustration of an orientation analysis system of a sixth variant of the laser system according to the exemplary embodiment;

FIG. 1N is a schematic illustration of an orientation analysis system of a seventh variant of the laser system according to the exemplary embodiment;

FIG. 1O is a schematic illustration of an orientation analysis system of an eighth variant of the laser system according to the exemplary embodiment;

FIG. 2 is a second perspective view of the laser system according to the exemplary embodiment, which is shown in FIG. 1A wherein the supporting arm is in a resting position.

FIG. 3 is a schematic view of the laser system of the exemplary embodiment which is shown in FIG. 1A and which illustrates the extent of a lateral movement range of the laser applicator of the laser system according to the exemplary embodiment, which is illustrated in FIG. 1A;

FIG. 4 is a further schematic top view of the laser system according to the exemplary embodiment which is shown in FIG. 1A;

FIG. 5 is a schematic side view of the supporting arm and the laser applicator of the laser system according the exemplary embodiment, which is shown in FIG. 1A;

FIG. 6 is a schematic view of information shown on a display device of the laser applicator of the laser system according to the exemplary embodiment, which is shown in FIG. 1A;

FIG. 7 is a schematic view of an imaging system, an OCT system and beam combiners which are arranged in the laser applicator of the laser system according to the exemplary embodiment, which is shown in FIG. 1A.

FIGS. 8A and 8B schematically illustrate the interaction measuring unit of the laser applicator of the laser system according to the exemplary embodiment, which is shown in FIG. 1A;

FIG. 9 is a schematic view of information shown on the display device of the laser applicator of the laser system according to the exemplary embodiment, which is shown in FIG. 1A;

FIG. 10 is a schematic view of a parallel linkage and a counterbalancing mechanism of the supporting arm of the laser system according to the exemplary embodiment, which is illustrated in FIG. 1A; and

FIGS. 11A to 11C are side views of the supporting arm and an articulated beam guide tube of the laser system and a coupling arrangement for coupling the articulated beam guide tube to the supporting arm of the laser system according to the exemplary embodiment;

FIG. 12 is a schematic illustration of the range of possible orientations of the laser applicator relative to the patient's head;

FIG. 13 is a schematic illustration of an image region within which the untreated eye of the patient appears in the image of the first imaging system;

FIG. 14 is a schematic illustration of a portion of the supporting arm of the ophthalmic laser system of the exemplary embodiment, which includes a controllable visual indicator;

FIG. 15A is a schematic perspective illustration of a portion of an intermediate joint of the supporting arm of the ophthalmic laser system of the exemplary embodiment, in a state in which the curved surface is removed from the intermediate joint; and

FIG. 15B is a schematic cross-sectional illustration of the portion of the intermediate joint which is shown in FIG. 15A.

### DETAILED DESCRIPTION OF EXEMPLARY EMBODIMENTS

For the purpose of promoting an understanding of the present invention, references are made in the text to exemplary embodiments of an ophthalmic laser system configured to perform treatments on an eye, only some of which are described herein. It should be understood that no limitations on the scope of the invention are intended by describing these exemplary embodiments. The inclusion of additional elements may be deemed readily apparent and obvious to one of ordinary skill in the art. Specific elements disclosed herein are not to be interpreted as limiting, but rather as a basis for the claims and as a representative basis for teaching one of ordinary skill in the art to employ the present invention in virtually any appropriately detailed apparatus or manner.

The term “substantially” or “approximately” as used herein may be applied to modify any quantitative representation that could permissibly vary without resulting in a change in the basic function to which it is related.

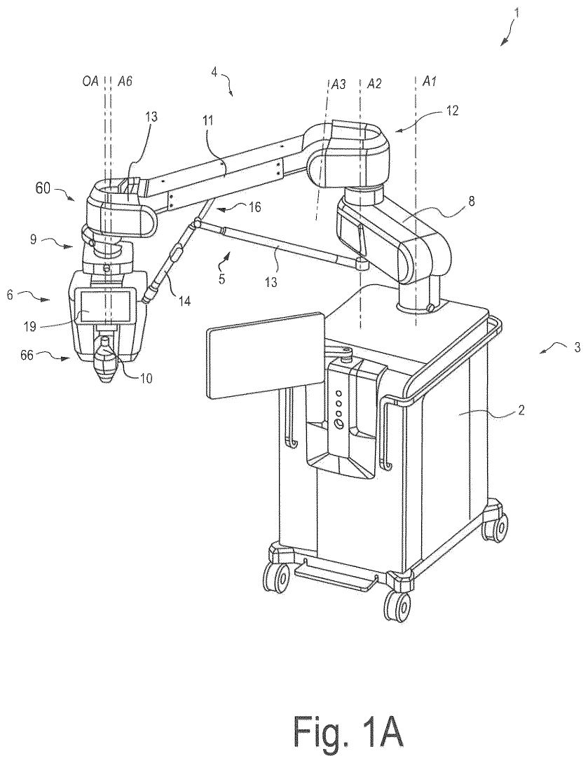

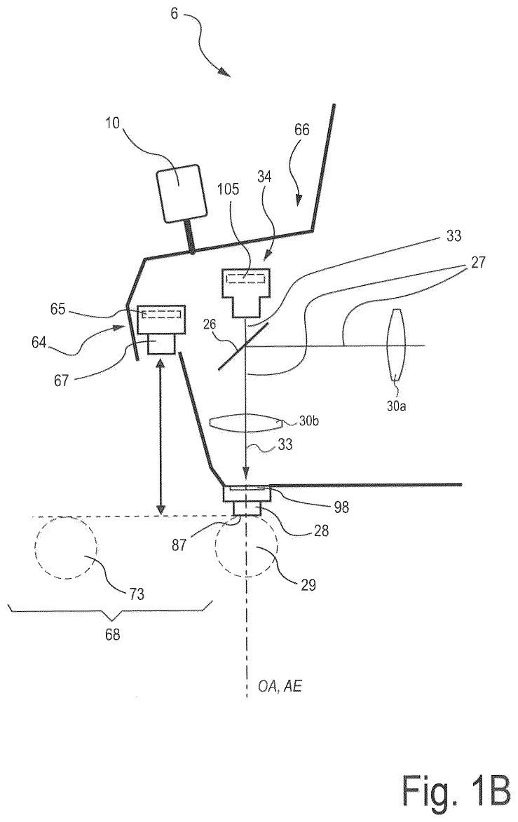

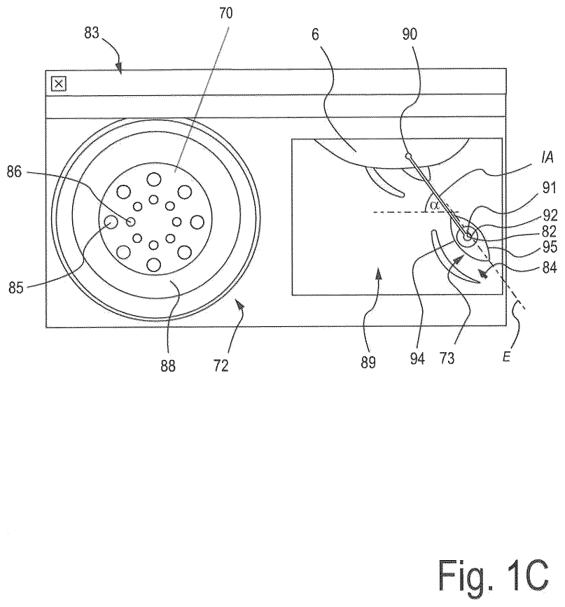

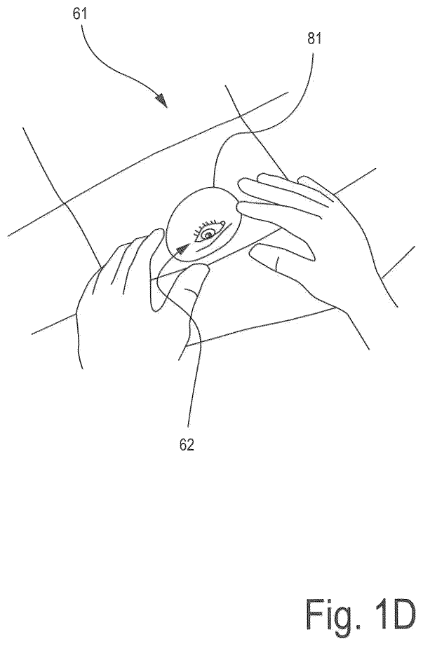

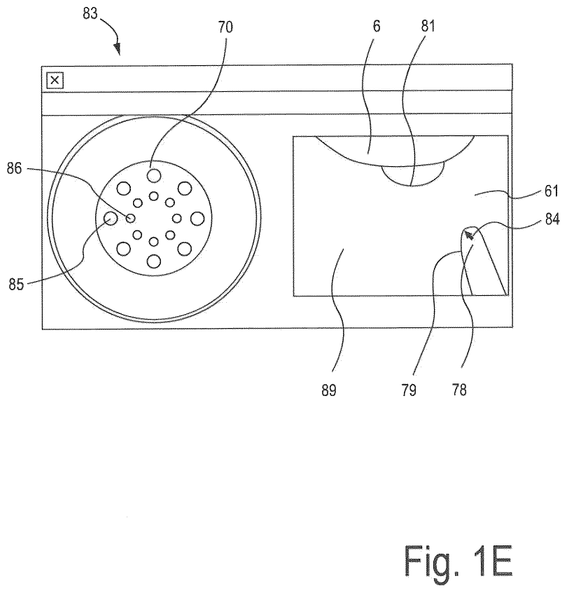

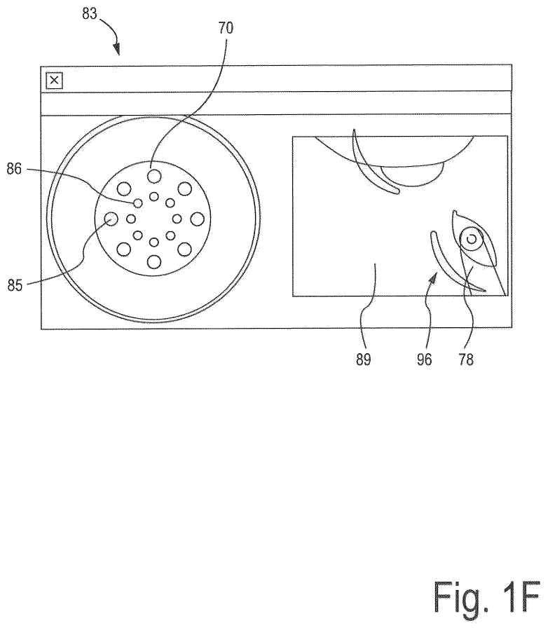

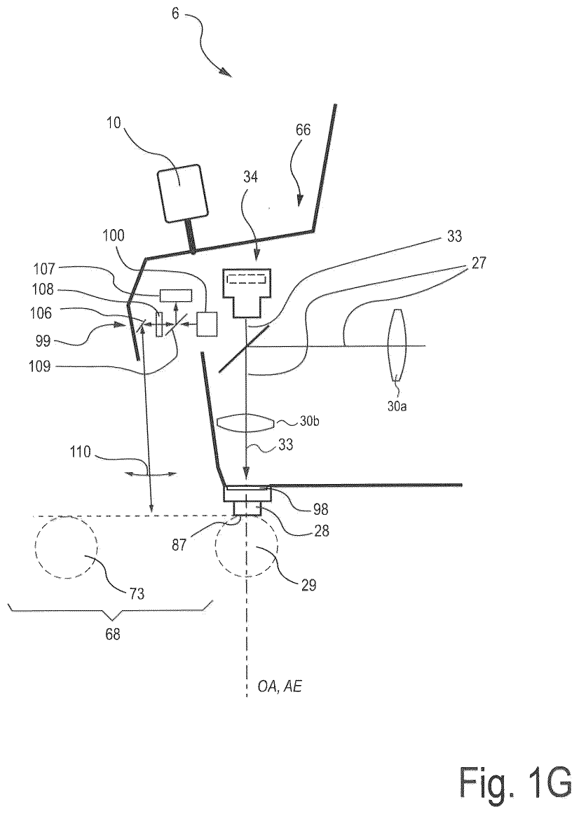

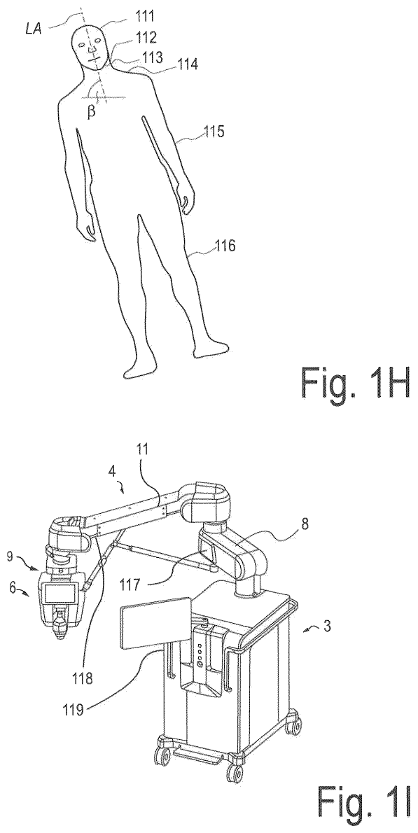

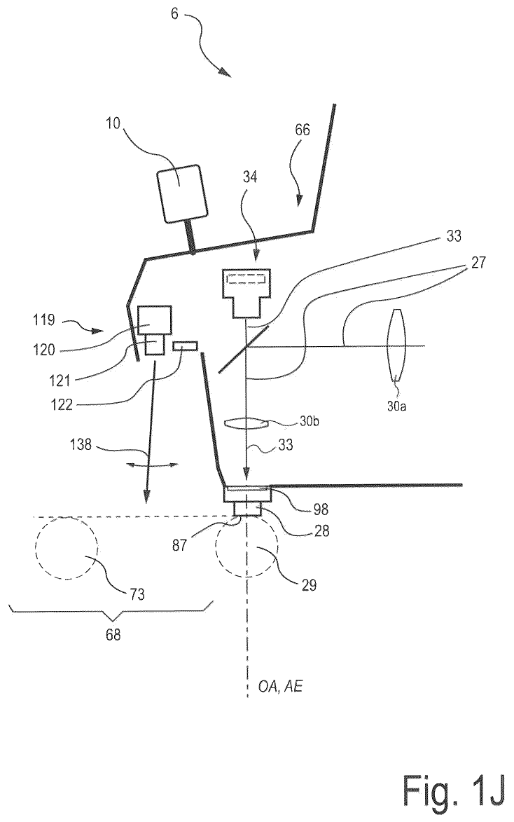

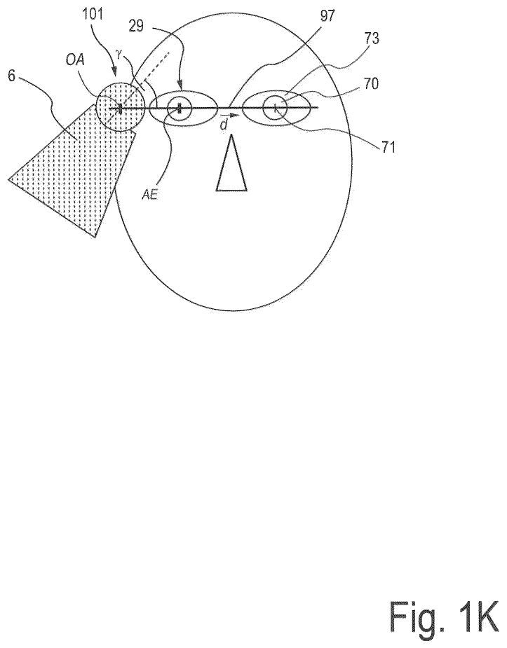

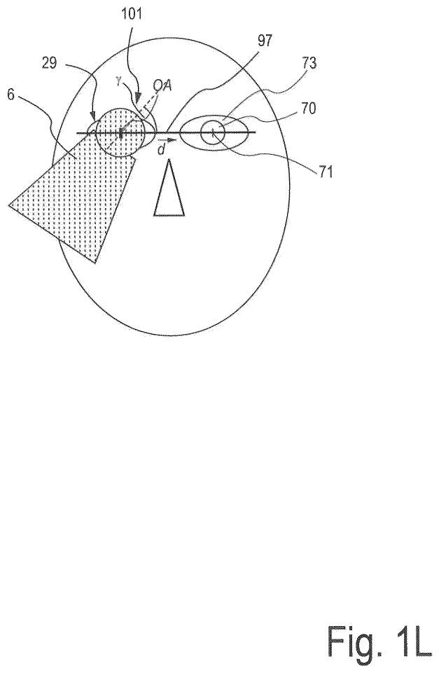

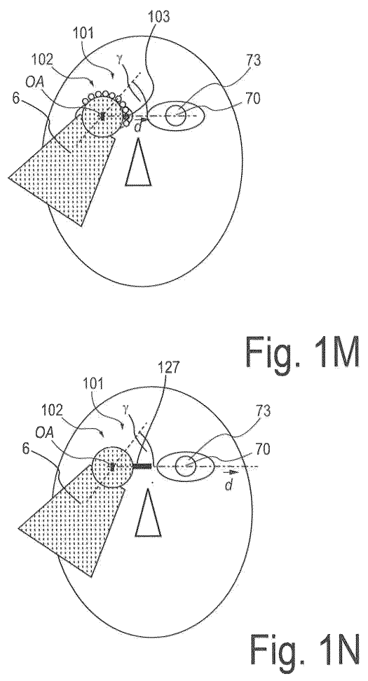

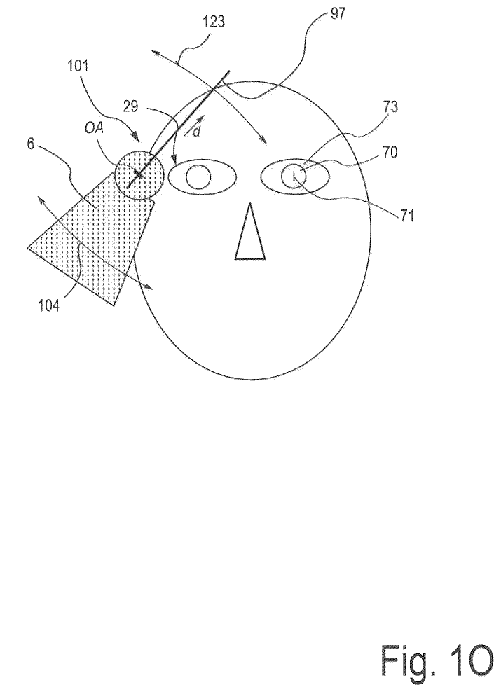

FIG. 1A is a schematic illustration of an ophthalmic laser system 1 according to an exemplary embodiment for performing laser treatments of an eye. The treatments may include, but are not limited to, forming flaps for laser assisted in situ keratomileusis (LASIK) treatments, forming corneal incisions and limbal relaxing incisions (LRI, AK), performing capsulotomy (in particular anterior capsulotomy) and lens fragmentation.

The laser system 1 includes a laser source, which is configured for generating a treatment laser beam for performing the laser treatments. At least a portion of the laser source is mounted within a housing 2 of a base 3. A further portion of the laser source may be arranged within a laser applicator 6, which is supported by an articulated supporting arm 4 and/or within a supporting arm 4, which supports the laser applicator 6. It is also conceivable that the base 3 includes more than one housing, wherein each of them houses a portion of the laser source. By way of example, a first housing of the base 3 houses an oscillator laser of the laser source and a second housing of the base houses an amplifier and/or a precompensator of the laser source.

The laser system 1 comprises a laser optical system which is configured to direct a laser beam towards the eye of a patient who is disposed on a patient bed or support, which is not illustrated in FIG. 1A. The laser source generates a treatment laser beam, which is guided by the laser optical system through a portion of a supporting arm 4, an articulated beam guide tube 5, and a laser applicator 6. The articulated beam guide tube 5 extends at least between a first location, where the laser beam exits from the supporting arm 4 and a second location, where the laser beam enters into the laser applicator 6. However, the present disclosure is not limited to such a configuration for guiding the treatment laser beam from the base 3 to the patient's eye. By way of example, the laser beam may be guided through the entire supporting arm. In particular, the laser system may be configured so that it does not include a beam guide tube. It is also conceivable that the articulated beam guide tube 5 extends at least between a first location, where the treatment laser beam exits from the base 3 to a second location, where the treatment laser beam enters into the supporting arm 4 or into the laser applicator 6. A portion of the articulated beam guide tube may be arranged within the base 3, within the supporting arm 4 and/or within the laser applicator 6.

In the exemplary embodiment, which is shown in FIG. 1A, the laser source is configured to emit a pulsed laser beam having a pulse energy and a pulse duration, which is sufficient to generate laser-induced optical breakdown (LIOB) within the cornea, the natural lens or within the lens capsule of the patient's eye. The laser-induced optical breakdown generated by a laser pulse leads to photodisruption, so that a series of consecutive overlapping or closely located laser pulses generate cuts within the corneal tissue, the natural lens or withn the lens capsule. Photodisruption is a nonthermal process. The laser optical system includes a scanning system, which is configured to scan a focus of the pulsed laser beam within the eye to form perforated cuts or continuous (i.e. non-perforated) cuts. In the exemplary embodiment, which is shown in FIG. 1A, the scanning system is arranged within the laser applicator 6. However, it is also conceivable that at least a portion of the scanning system is arranged within the base 3 and/or within the supporting arm 4.

It is noted that the present disclosure is not limited to the above laser treatments and laser systems. Specifically, the laser system may be configured to controllably ablate corneal tissue without causing significant damage to adjacent and/or underlying tissues of the eye. The laser system may emit light having a wavelength greater than 150 nm or greater than 190 nm. The wavelength may be less than 400 nm or less than 200 nm. By way of example, the laser source may be configured as an excimer laser source. The laser system may be an argon-fluorine (ArF) excimer laser, which generates pulses of laser light having a wavelength of substantially 193 nm. The laser ablation process may be used for reshaping the cornea. Such ablative treatments may include, but are not limited to, photorefractive keratectomy (PRK), laser assisted subepithelial keratomileusis (LASEK), laser-assisted in-situ keratomileusis (LASIK) and phototherapeutic keratectomy (PTK). In each of these procedures, the laser beam may be used to remove a predetermined amount of the corneal stroma which is located beneath the corneal epithelium and Bowman's membrane to form a reshaped surface portion.

In the exemplary embodiment shown in FIG. 1A, the laser source is arranged in the base. However it is also conceivable that only a portion of the laser source is arranged in the base and second portion of the source is arranged in the laser applicator 6 or in the supporting arm 4. By way of example, an oscillator laser of the laser source may be arranged in the base and the regenerative amplifier and/or a precompensator of the laser source may be arranged in the laser applicator and/or supporting arm.

Using the supporting arm 4, the laser applicator 6 is positionable in three dimensions. The supporting arm 4 may be configured to allow manual positioning of the laser applicator 6 in three dimensions. However, it is also conceivable that the supporting arm may include one or more motors so that at least a portion of the movements, which can be carried out using the supporting arm 4, are motorized. Between the supporting 4 and the laser applicator 6, a three-axis motorized positioning system 9 is arranged. The positioning system 9 may be configured so that the laser applicator 6 is positionable relative to the supporting arm 4. It is also conceivable that the positioning system 9 is part of the supporting arm 4 so that the laser applicator and a portion of the supporting arm 4 is positionable relative to a further portion of the supporting arm 4. By way of example, the positioning system 9 may be arranged between two arm segments or may be part of an arm segment of the supporting arm 4.

By way of example, as is described in more detail below, the positioning system 9 is used for fine positioning procedure after the laser applicator 6 has been positioned in a course positioning procedure using the supporting arm 4.

The positioning system 9 may be operatively coupled to a controller (not shown in FIG. 1A). The controller may be arranged within the base 3. However, it is also conceivable that the controller is arranged in a housing which is separate from the base 3 and also separate from the laser applicator 6. The controller may be connected to the base 3, to the positioning system 9 and/or components of the laser applicator 6 via a wired or wireless connection.

The laser applicator 6 may further include a manually operable control unit 10, which is operatively coupled to the controller for performing the positioning of the laser applicator 6 relative to the portion or relative to the entire the supporting arm 4 using the positioning system 9. The manually operable control unit 10 may be configured for directional control. By way of example, the control unit 10 may be configured as a joystick (such as illustrated in FIG. 1A). However, the present disclosure is not limited to such a control element. It is also conceivable that the control element includes one or more buttons, each of which representing a direction of travel of the laser applicator 6.

The configuration of the ophthalmic laser system according to the exemplary embodiment allows positioning the laser applicator relative to the patient, so that the patient can remain in the regular patient bed where the patient has received pre-surgical treatment. Thereby, patient transfers can be avoided, which otherwise would be required for moving patients from their regular patient beds to a separate stationary patient support which is part of the laser system and specifically provided for conducting the laser surgical steps.

For this reason, the surgical laser system according to the present disclosure not only increases the effectiveness of the clinical workflow but also reduces the footprint required in the surgical operation theatre, since there is no space necessary for a fixedly installed patient support, which is provided solely for performing the laser surgery. Further, a movable laser surgical system facilitates cleaning and disinfection of the surgical operation theatre.

Moreover, in order to reduce footprint and costs, it is desirable that femtosecond laser systems, which are configured for cataract surgery also be used for forming corneal flaps for laser in situ keratomileusis (LASIK). A femtosecond laser for forming LASIK flaps, however, should be located close to the excimer laser system in order to make it easy for the patient to move from one system to the other. On the other hand, a femtosecond laser for cataract surgery, should be located in the sterile operating theatre, where the natural lens is emulsified using a phacoemulsification device and where the intraocular lens is implanted. The laser system according to the exemplary embodiment is a mobile laser system, which can easily be moved between different locations in the hospital. Also, the articulated supporting arm allows the surgeon to more flexibly arrange the laser system within the surgical operating room, where many other devices, such as a surgical microscope, a phacoemulsification system and operating room trolleys are located and where there must be sufficient space for one or more surgeons and other medical staff members.

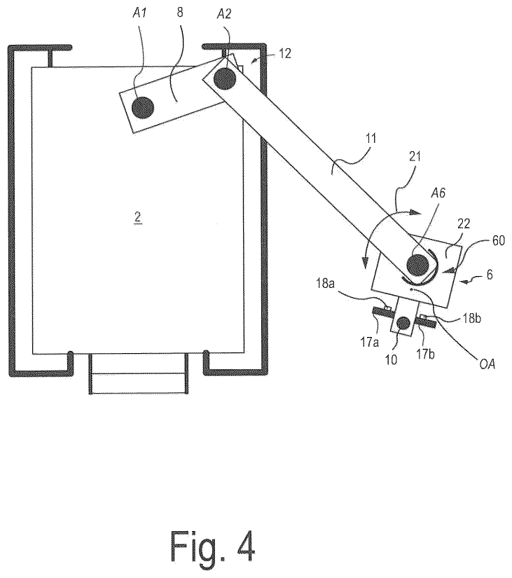

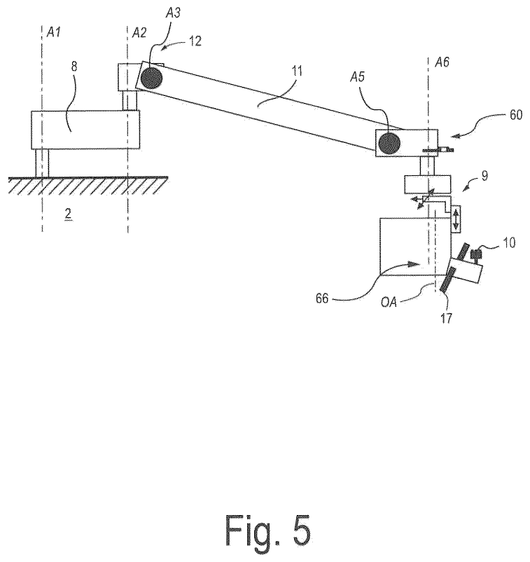

As is illustrated in FIG. 1A and also in the schematic top view of FIG. 4 and the schematic side view of FIG. 5, the supporting arm 4 may be configured as an articulated supporting arm having to or more arm segments, which are connected in series. The supporting arm 4 may include a first arm segment 8 and a second arm segment 11. The first arm segment 8 may be rotatably supported by the base 3 so that the first arm segment 8 is rotatable about a first vertical axis A1. The first vertical axis A1 may have a fixed position and orientation relative to the base 3. Additionally or alternatively, the base may be configured so that position, in particular a height and/or orientation (rotation) of the first arm segment 8 relative to the base 3 is adjustable. By way of example, the base 3 may be configured so that the first arm segment is supported by the base using a rotational bearing assembly (not shown in FIG. 1A) for rotatably supporting the first arm segment 8 about the first vertical axis A1. The base 3 may be configured so that a height of the rotational bearing assembly relative to remaining portions of the base 3 is adjustable, e.g. using a motorized heigh adjusting mechanism.

The first arm segment 8 may be connected to the second arm segment 11 via an intermediate joint 12. The intermediate joint 12 may be configured so that an orientation of the second arm segment 11 relative to the first arm segment 8 is adjustable in two dimensions. By way of example, the intermediate joint 12 may be configured so that the second arm segment is rotatable about a vertical axis A2 and also rotatable about a horizontal axis A3.

The supporting arm 4 is configured so that before and after a rotation of the second arm segment 11 about the horizontal axis A3, the laser applicator 6 has a same vertical orientation. In the exemplary embodiment, the rotation of the second arm segment 11 about the horizontal axis A3 is coupled with an orientation of the laser applicator 6 relative to the second arm segment. The coupling is a mechanical coupling which is obtained through a multi bar linkage of the supporting arm 4, which may be configured as a parallel linkage. Two or more parallel bars of the parallel linkage may form the second arm segment 11. However, is is also conceivable that the second arm segment 11 and/or the laser applicator 6 include an inclination sensor for measuring an inclination of the second arm segment 11 and/or the laser applicator 6 relative to the horizontal plane. A controller may be provided which receives output signals from the one or more inclination sensors and which controls an adjustment of the vertical orientation of the laser applicator 6 based on the measured inclination. The adjustment of the vertical orientation may be performed using a motor which drives a rotational joint. It is also conceivable that the connection between the second arm segment 11 and the laser applicator 6 is configured so that the vertical orientation of the laser applicator 6 is maintained by gravitational forces acting on the laser applicator 6.

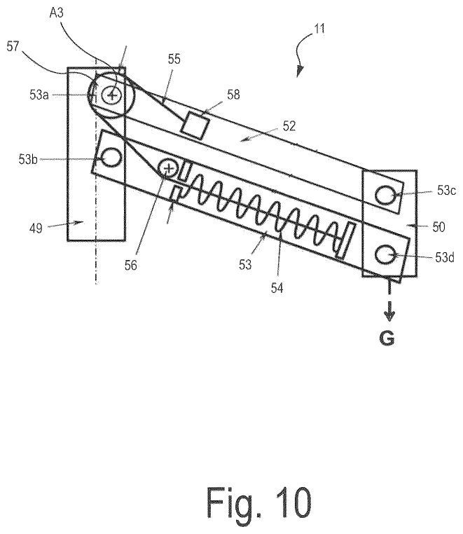

The parallel linkage may be configured so that a distal end of parallel linkage (i.e. distal relative to the base 3) maintains its vertical orientation, irrespective of the orientation of the second arm segment 11 relative to the horizontal plane. An example for a parallel linkage is described below with reference to FIG. 10.

As can be seen from FIGS. 1A, 4 and 5, the laser applicator 6 can be rotated relative to the supporting arm 4 about a vertical rotation axis A6. The vertical rotation axis A6 may extend through the laser applicator 6, in particular through a housing 22 of the laser applicator 6. However, it is also conceivable that the vertical rotation axis A6 does not extend through the laser applicator 6. Rotating the laser applicator about the vertical rotation axis A6 changes an orientation of the laser applicator 6 relative to the supporting arm 4.

Rotating the laser applicator 6 relative to at least a portion of the supporting arm 4 allows the surgeon during the coarse positioning procedure to adjust the orientation of the laser applicator 6 so that the positioning of the laser applicator 6 relative to the patient's eye is not hindered by space constraints defined by the patient's anatomy. Additionally or alternatively, the laser system may be configured so that the laser applicator 6 can be rotated relative to the supporting arm about a horizontal axis relative to at least a portion of the supporting arm (not shown in FIGS. 4 and 5). In addition to the laser applicator 6 being rotatable about the horizontal axis, the laser system may also be configured so that the laser applicator 6 is rotatable about a roll axis, which is stationary relative to the laser applicator 6 i.e. stationary for different rotational positions of the laser applicator 6 obtained by the rotation about the vertical and/or horizontal axes (not shown in FIGS. 4 and 5). This provides an even higher flexibility for adjusting the orientation of the laser applicator 6 relative to the patient.

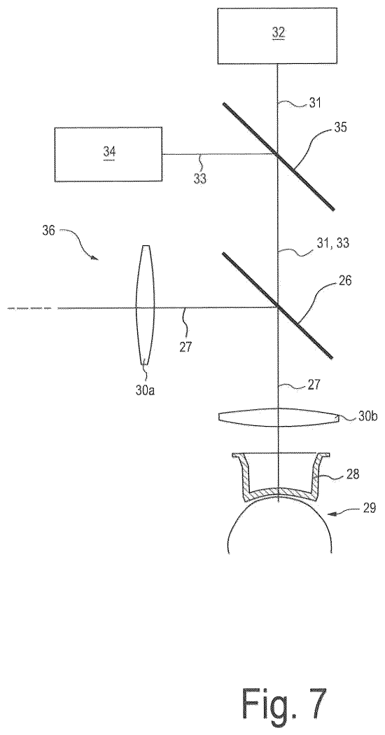

The supporting arm 4 of the laser applicator 6 of the laser system includes a rotational guide 60 having a vertical rotation axis A6, which is parallel or substantially parallel to the optical axis OA of the beam delivery system within the laser applicator 6 at a location, where the treatment laser beam exits from the laser applicator 6 to travel toward the contact element (designated with reference numeral 28 in FIG. 7) of the patient interface. The rotational guide may have a single rotational degree of freedom. As can be seen from FIGS. 1A and 5, in the exemplary embodiment, the rotational guide 60 rotates the laser applicator 6 and the motorized three axis positioning system 9 about the vertical axis A6. However, it is also conceivable that the rotational guide 60 is arranged between the motorized three axis positioning system and the laser applicator 6 so that the rotation axis A6 has a fixed position relative to the optical axis OA of the beam delivery system. This allows aligning the vertical rotation axis A6 with the optical axis OA so that the laser applicator is rotatable about the optical axis OA.

The rotational guide 60 allows moving the laser applicator 6 to adjust an orientation of the laser applicator 6 about the vertical axis to different orientations of the patient's head measured around the axis of the eye to be treated. The movement may be performed manually by the user or automatically by a controller of the ophthalmic laser system, which controls a drive system (not shown in FIG. 1A) which is configured to rotate the laser applicator 6 about the rotation axis A6.

The ophthalmic laser system of the exemplary embodiment which is illustrated in FIG. 1A does not have an integrated patient support as in the prior art devices, i.e. patient supports, which have predefined positions and/or orientations relative to the laser system. Rather, the laser system of the exemplary embodiment has a supporting arm 4, which allows performing laser treatments when the patient is lying on a regular surgical bed. Due to this flexibility, however, the orientation of the laser applicator 6 relative to the patient's head around the axis of the eye to be treated, is unknown after the surgeon has positioned the laser applicator close to the patient's using the positioning arm. Also, the surgeon typically uses handles provided at the laser applicator when he or she manually performs the coarse positioning procedure using the supporting arm 4. Therefore, during the coarse positioning procedure, the orientation of the laser applicator is typically changed to an unknown orientation.

However, due to the anatomical shape of the patient's head on the one hand and the shape of the outer surface of the housing of the laser applicator 6 on the other hand, the laser applicator can only dock to the patient's eye in specific orientations relative to the patient's head. This can make the process of docking the eye of the patient laborious and time-consuming.

The inventors have shown that this drawback can be overcome by providing an orientation analysis system, which acquires orientation-related data or by providing an orientation indication system, which indicates to the user the relative orientation of the laser applicator with respect to the patient's head. The orientation-related data can also be used for determining the scanning path of the focus of the treatment laser beam within the patient's eye or on an anterior surface of the cornea.

The orientation-related data are acquired using one or more body portions of the patient and/or one or more objects, which have a fixed position and/or orientation relative to a body portion of the patient. It has been shown that this allows efficient collision avoidance between the laser applicator and the patient's head.

As will be explained in more detail further below, the orientation-related data, which are acquired from the one or more body portions and/or objects allow the user and/or a controller of the ophthalmic laser system (not shown in FIG. 1A) to position the laser applicator so that during the laser treatment, the laser applicator has an orientation about the axis of the eye to be treated, which (i) corresponds or substantially corresponds to a pre-determined target orientation; or which (ii) is within or substantially within a pre-determined target range. The target orientation and/or the target range may depend on the outer shape of the housing of the laser applicator.

It is noted that depending on the shape of the outer surface of the housing of the laser applicator 6, a comparatively broad pre-determined target range can be sufficient. For the same reason, it can be sufficient to determine only one or more (i.e. not all) parameters, which are indicative of the orientation of the patient's head. It can even be sufficient to determine one or more parameters which are only correlated with the orientation.

As is explained in more detail in the following paragraphs, in a first and a second variant of the orientation analysis system of the exemplary embodiment, the ophthalmic laser system includes one or more 2D imaging systems. The 2D imaging systems may be configured for imaging one or more of the body portions and/or objects onto an image sensor of the imaging system.

FIG. 1B is a schematic cross-sectional illustration of a lower portion 66 (also shown in FIG. 1A) of the laser applicator 6, and a contact element 28 of the patient interface via which the beam delivery system for delivery of the treatment laser beam 27 is coupled to the patient's eye to be treated 29. The laser applicator 6 includes a first imaging system 64. The first imaging system 64 includes an imaging optical system 67, which is configured to image, onto an image sensor 65, one or more body portions and/or objects which are within an object field 68 of the first imaging system 64. As is explained further below, based on the image of this first imaging system, it is possible to determine an orientation of the laser applicator 6 relative to the patient's head about the axis of the eye to be treated.

The laser applicator 6 also includes a second imaging system 34, which is configured for acquiring a frontal image of a central portion of the eye to be treated, such as the pupillary opening and at least a portion of the iris, by imaging the central portion onto an image sensor 105 of the second imaging system. As can be seen form FIG. 1B and as is described in more detail with reference to FIG. 7, an imaging beam path of the second imaging system 34 traverses a portion 30b of the beam delivery system.

The images which are acquired using the second imaging system 34 may be used for aligning the optical axis OA of the beam delivery system 6 with the axis AE of the eye to be treated. This is explained in more detail below with reference to FIG. 1C. Specifically, in order to acquire the orientation-related data, the first imaging system 64 may acquire one or more images when the optical axis OA of the beam delivery system is aligned with the axis AE of the eye to be treated. On the other hand, it has been shown by the inventors that a sufficient accuracy in the determination of the orientation can be obtained if images of the first imaging system 64 are used without the optical axis OA of the beam delivery system being in alignment with the axis AE to be treated.

Additionally or alternatively, in the ophthalmic laser system of the exemplary embodiment, the images which have been acquired using the second imaging system 34 may be used for determining a pupil center of the eye to be treated in order to center a scanning pattern which is used for the laser treatment. Additionally or alternatively, the controller of the ophthalmic laser system may be configured to compare images acquired with the second imaging system with one or more images, which have been acquired using a diagnostic device (e.g. a wavefront analysis system or an OCT system). This allows determination of the scanning paths for performing the laser treatment based on measurements of the diagnostic device. The comparison may include iris registration and/or limbal blood vessel registration.

FIG. 1C is a schematic illustration of a window 83 of a graphical user interface of the ophthalmic laser system of the exemplary embodiment. In the left-hand portion of the window 83, the graphical user interface displays an image 72, which has been acquired using the second imaging system 34 (shown in FIG. 1B). The image 72 shows a central portion of the eye to be treated. The imaged central portion includes the pupillary opening 70 and at least a portion of the iris 88. As can be seen from FIG. 1B, an imaging beam path 33 of the second imaging system 34 extends through a portion 30b of the beam delivery system and through a contact element 28 of the patient interface. The imaging beam path 33 of the second imaging system 34 is combined with the beam path 27 of the treatment laser using a beam combiner 26.

The image 72 also shows an inner ring of eight Purkinje reflexes 86, which are generated by reflections of a light of a ring of LED light sources (shown in FIG. 1B and designated with reference numeral 98) at the anterior surface of the cornea. The schematic illustration of image 72 also shows an outer ring 85 of eight reflections of light of the LED light sources, which are generated by reflections of the light at the contact surface (designated with reference numeral 87 in FIG. 1B) of the contact element 28. If the contact element 28 is in contact with the cornea, the inner ring 86 of Purkinje reflexes disappears.

The inner ring of reflections 86 and the outer ring of reflections 85 can be used to align the optical axis OA of the laser applicator 6 with the axis AE of the eye to be treated 29 using the three-axis positioning system 9 (described above in connection with FIG. 1A) of the ophthalmic laser system, which allows positioning of the laser applicator 6 relative to the supporting arm 4. Specifically, based on the position of the inner ring of Purkinje reflexes 86 relative to the outer ring 85 of reflexes, the user can control the motorized three-axis positioning system 9 (shown in FIGS. 1A and 5) using the manually operable control unit 10 (shown in FIGS. 1A and 1B) until both rings 85, 86 are concentric, as is shown in FIG. 1C. If both rings 85, 86 are concentric or substantially concentric, the optical axis OA of the laser applicator is aligned or substantially aligned with the axis AE of the eye to be treated.

Therefore, since in the image 72, the inner and outer ring of circular reflections 85, 86 are concentric and the inner ring of Purkinje reflexes 86 are visible, this indicates that the optical axis of the beam delivery system is aligned with the axis of the eye and the contact element of the patient interface is not fully in contact with the anterior surface of the cornea. In FIG. 1C, the frontal image 72 of the eye to be treated may, for example, relate to a distance between the contact surface 87 of the contact element 28 and the anterior surface of the cornea of between 10 millimeters one one hand and 0 millimeter (i.e. in contact with the cornea) or substantially 0 millimeter. The distance may be measured along the optical axis.

In a first variant of the exemplary embodiment, which is described with reference to FIG. 1C, the controller determines one or more parameters of the orientation of the laser applicator relative to the patient's head using the alignment or substantial alignment between the optical axis of the laser applicator (designated with reference numeral OA in FIG. 1B) and the axis AE of the eye to be treated. In the right-hand portion of window 83, which is shown in FIG. 1C, the graphical user interface displays at least a portion of an image generated by the first imaging system 64 (shown in FIG. 1B) when both axes are aligned as indicated by the frontal image 72 of the eye to be treated shown in the left portion of window 83. Due to the alignment between both axes, the position 90 of the pupil center of the eye to be treated relative to the image 89 of the first imaging device is known. The reason for this is that the first imaging system 64 (shown in FIG. 1B) has a fixed position and orientation relative to the optical axis of OA of the beam delivery system. Therefore, it is possible to determine the orientation of the laser applicator relative to the patient's head, measured about the axis of the eye to be treated based on a determined location of the pupil center 91 of the untreated eye in the image 89 of the first imaging device. The pupil center 91 is therefore an anatomical landmark based on which the interpupillary axis, i.e. an anatomical feature of the patient's body can be determined. It is noted that although in FIG. 1C, the position of the pupil center 90 of the eye to be treated is located within the image 89, it is also conceivable that the position 90 of the pupil center of the eye to be treated is outside the image 89.

The graphical user interface of the ophthalmic laser system of the first variant is configured to receive user input via a pointing device, such as a computer mouse, wherein the user input identifies one or more locations in the image 89 of the first imaging system, which correspond, substantially correspond, are indicative of or are substantially indicative of the pupil center 91 of the untreated eye. By way of example, the user can position, using the pointing device, a pointer 84 in the image 89 of the first imaging device at a location, which corresponds or substantially corresponds to the pupil center 91 of the untreated eye 73. It is also conceivable that the graphical user interface is configured so that, using the pointing device, the user can specify a boundary in the image 89 of the first imaging device, which corresponds or substantially corresponds to the pupillary edge 92. The image region which is thereby specified is indicative or substantially indicative of the pupil center 91 of the untreated eye 73. The controller may be configured to determine, based on the user input, which specifies the pupillary edge, a location in the image 89, which corresponds to the center of the pupil. Moreover, it is further conceivable that the user specifies a location within the pupillary opening 82 of the untreated eye and the controller executes an image processing algorithm, which determines an image region, which corresponds to the pupillary opening, e.g. by using an edge detection filter. Based on the determined pupillary opening, the controller determines the pupil center of the untreated eye.

Depending on the outer shape of the housing of the laser applicator, it is conceivable that any point within the pupillary opening 82, i.e. without determination of the pupil center 91 of the untreated eye, can be used for determining the rotation angle of the laser applicator with sufficient accuracy.

Based on the location of the pupil center 91 of the untreated eye in the image 89 of the first imaging device and the known position 90 of the pupil center of the eye to be treated relative to the image 89, the controller can determine the interpupillary axis IA of the patient's head, which corresponds to a lateral axis (i.e. a left-right axis) of the head. Therefore, two anatomical landmarks (i.e. two pupil centers) are used for determining an anatomical feature of the human body (i.e. the interpupillary axis IA).

The controller is further configured to determine, one or more parameters of the orientation of the interpupillary axis IA of the head relative to the image 89 of the first imaging device, such as the angle a illustrated in FIG. 1C, which corresponds to the orientation of the laser applicator relative to the patient's head measured about the treatment axis of the eye. The controller is further configured to generate data representative of graphical and/or textual information using the determined one or more parameters. The controller may further be configured to output, using a user interface of the ophthalmic laser system, the graphical and/or textual information to the user. The user interface may include a display and/or a graphical user interface. By way of example, the controller may output a value, which is indicative of the orientation of the interpupillary axis IA relative to the image 89 on a display of the ophthalmic laser system.

In a second variant of the exemplary embodiment, the controller determines, automatically or based on user input, the orientation of the laser applicator relative to the patient's head without aligning the optical axis OA of the beam delivery system (designated with reference numeral OA in FIG. 1B) with the axis AE of the eye to be treated.

By way of example, in the second variant of the exemplary embodiment, the controller executes an algorithm which is configured to determine, automatically or based on user input, an orientation of the untreated eye 73 in the image 89 which has been acquired by the first imaging system. It is also conceivable that the orientation of the treated eye is determined.

By way of example, the controller may execute an image processing algorithm which is configured to determine borders of the upper and lower eye lids 94, 95 in the image 89 of the first image device, e.g. using an edge detection filter. The controller may further be configured determine the orientation of the interpupillary axis IA of the patient's head based on the detected borders of the upper and lower eyelids 94, 95. By way of example, the controller may be configured to use an anatomical model, which provides a correlation between the orientation of the interpupillary axis IA and the horizontal axis E of the eye between endocanthion and exocanthion, which is defined by the the upper and lower eyelids 95.

It has been shown by the inventors that such a determination of the orientation of the laser applicator relative to the patient's head results in an orientation angle, which has a sufficient accuracy.

Based on the determined orientation, the user and/or controller can adjust the orientation of the laser applicator about the rotation axis A6 (shown in FIG. 1A) of the laser applicator so that the orientation corresponds substantially corresponds to a target orientation or is within or substantially within a pre-determined target range.

The ophthalmic laser system may be configured so that, using the relative orientation, the user can manually rotate the laser applicator about the rotation axis of the rotational guide 60. The ophthalmic laser system may include a braking and/or locking system for arresting a rotary movement of the laser applicator about the rotation axis A6 using the rotational guide 60. The ophthalmic laser system may include a rotational position sensor (not illustrated) for measuring a rotational position of the laser applicator relative to the distal end of the supporting arm. The rotational position sensor may include encoder, such as a mechanical, optical, magnetic, and/or electromagnetic induction type encoder. The ophthalmic laser system may further be configured to indicate to the user, using an output of the rotational position sensor and further using the determined orientation-related data, when the orientation of the laser applicator relative to the patient's head corresponds or substantially corresponds to the pre-determined target orientation or is within or substantially within the pre-determined target range.

Additionally or alternatively, the ophthalmic laser system may include a drive system (not illustrated) for rotating the applicator about the rotation axis A6 using the rotational guide 60. The controller may be operatively coupled with the drive system to control the rotation based on the determined orientation of the laser applicator relative to the patient's head. The controller may also be coupled to the rotational position sensor for measuring the orientation of the laser applicator relative to the distal end of the supporting arm.

As has been explained above with reference to FIG. 1A, the rotation axis A6 of the laser applicator and the optical axis OA of the laser applicator are parallel but non-aligned. Therefore, rotating the laser applicator so that that laser applicator has the pre-determined target orientation or is within the pre-determined target range, causes a displacement of the optical axis OA of the laser applicator relative to the axis AE of the eye to be treated.

This displacement can be adjusted using the three-axis positioning system (designated with reference numeral 9 in FIG. 1A). Since the rotational guide 60 is connected to the laser applicator 6 via the three-axis positioning system 9, a displacement of the position of the laser applicator 6 using the three-axis positioning system 9 does not change the adjusted orientation of the laser applicator 6 relative to the patient's head.

As is explained in the following paragraphs, the ophthalmic laser systems of the first and second variants are further configured to determine the orientation of the laser applicator relative to the patient's head even during surgical treatments where the untreated eye cannot be imaged by the first imaging system since it is covered by drapes, or other medical coverings used in the operating room, e.g. to prevent infections. By way of example, as is illustrated in FIG. 1D, during cataract surgical procedures, the patient's head is typically covered by a sterile drape 61 having an opening 81 leaving only the eye to be treated 62 exposed.

As can be seen from the schematic illustration of the window 83 of the graphical user interface in FIG. 1E, due to the sterile drape 61, the untreated eye cannot be seen in the image 89 of the first imaging system. However, the ophthalmic laser system of the fist and second variants may be configured so that the orientation of the laser applicator relative to the patient's head can, additionally or alternatively be determined based on an object, which has a fixed position and/or orientation relative to a body portion of the patient. The object may be attached to the patient's body and/or the sterile drape or may be loosely placed at a fixed position and/or orientation relative to the body portion.

By way of example, as can be seen from the image of the first imaging system 89 in FIG. 1E, the surgeon can place an object, such as his or her finger 78, on the sterile drape 61 so that the position and/or orientation of the finger 78 is an indicator for the position and/or orientation of a body portion of the patient (such as the untreated eye 73), which is covered by the drape 61. By using his or her finger, the surgeon can also verify the correct position of the finger 78 by palpating the untreated eye under the drape 61. It is also conceivable that other objects, in particular non-body objects, are used for indicating the position and/or orientation of one or more body portions of the patient. The objects may be attached to or loosely placed on the drape 61. By way of example, it is possible that the ophthalmic laser system includes objects, which have one or more marks. An image processing algorithm, which is executed by the controller, may be configured to detect a position and/or orientation of the one or more marks. It is also conceivable that a plurality of marks are provided at an object and the controller is configured to determine, based on a 2D image of the marks, all orientational degrees of freedom of the object. By way of example, the marks are provided at the eye fixation device and a 2D image of the eye fixation device is used for determining all orientational degrees of freedom of the suction clip.

The one or more marks may be indicative of a position and/or orientation of a body portion of the patient. The marks can also help the user to indicate, using the cursor 84 of the pointing device, the position and/or orientation of the object in the image. The marks may be infrared marks, in particular infrared absorbing or reflecting marks. Such marks are independent of the skin color of the patient and environmental lighting conditions. One or more marks may be provided at the eye fixation device, which serves as one of the objects for acquiring the orientation-related data.

It is noted that even when the patient's head or body is not covered by a drape 61, objects can still be used to indicate the position and/or orientation of body portions of the patient. Specifically, the objects, in particular the marks, which are provided at the objects, can facilitate automatic or user-based detection of the position and/or orientation of the objects within the image 89 of the first imaging device.

Returning to the example, which is illustrated in FIG. 1E, the ophthalmic laser system of the exemplary embodiment may be configured to determine, automatically or based on user input, a position and/or an orientation of the object (such as the finger 78 of the surgeon) in the image 89 acquired by the first imaging system. By way of example, the controller may be configured to execute an image processing algorithm, which identifies one or more features of the object, such as a boundary 79 of the image portion, which corresponds to the object, such as the surgeon's finger 78. By way of example, the image algorithm may perform a segmentation of at least a portion of the image 89. The controller may be further be configured to determine an image region, which corresponds to a distal portion of the surgeon's finger 78, such as the distal segment of the finger. The controller may be configured to use typical anatomical values for the length of the distal segment of the finger. Additionally or alternatively, the controller may be configured to determine an orientation of the surgeon's finger in a plane which is perpendicular to the axis of the eye to be treated.

Additionally or alternatively, the ophthalmic laser system may be configured so that user can identify, using the cursor 84 of a pointing device, such as a computer mouse, at least a portion of the boundary 79 of the surgeon's finger 78 in the image.

As is schematically illustrated in FIG. 1F, in order to assist the surgeon in verifying the position of the untreated eye, which has been determined automatically or based on user input, the ophthalmic laser system may be configured to generate, based on the determined location of the untreated eye, an overlay which is superposed on the image 89 of the first imaging system and which includes one or more symbolic representations of features of the eye, such as a symbolic representations of the eyebrows the upper and lower eyelids and the pupillary edge. It has been shown by the inventors that such symbolic representations of features of the patient's face make it easier for the surgeon to check, whether the determined orientation of the patients head relative to the laser applicator has the required accuracy.

In each of the above-described variants of the exemplary embodiment, it is conceivable that the controller executes an algorithm, which has been generated using machine-learning techniques in order to determine the location and/or orientation of one or more body portions and/or objects in the image.

In the above-described variants of the exemplary embodiment, the first and second imaging systems (designated with reference numerals 64 and 34 in FIG. 1B) may include an image sensor, which is sensitive to electromagnetic radiation in the visible and/or infrared wavelength range. In the infrared wavelength range or for wavelengths within a range of between 700 nanometers and 2,500 nanometers, pupils reflect almost all the infrared light they receive along the path back to the camera, producing the so-called “bright pupil effect”. If illuminated off the camera optical axis, the pupils appear dark since the reflected light will not enter the camera lens. This produces the so-called dark pupil effect. Therefore, using infrared light, it is possible to determine the pupil center with a higher accuracy in the images of the first and second imaging systems 64 and 34.

Additionally or alternatively, based on an image, which has been acquired at wavelengths in the visible and/or infrared wavelength range, the controller of the ophthalmic laser system may be configured to determine, at least a portion of an outer contour of the imaged patient's head. The controller may be configured to determine, automatically or based on user input, the orientation of the applicator relative to the patient's head based on the determined portion of the outer contour. It is also conceivable that the controller executes an algorithm, which has been generated using machine-learning techniques to interpret the image data of the infrared image.

FIG. 1G schematically illustrates a third variant of the exemplary embodiment. In the third variant of the exemplary embodiment, the rotation analysis system includes a giga-terahertz imaging system 99, which images at least a portion of the patient's head using gigahertz and/or terahertz radiation having frequencies in a range of between 10 GHz and 100 THz, in particular between 30 GHz and 50 THz.

The giga-terahertz imaging system 99 includes a radiation source 100 for generating a collimated beam of gigahertz-and/or terahertz radiation, a focusing mirror system 108 and a scanning mirror 106 for scanning the beam across the surfaces of the body portions and/or objects (schematically illustrated in FIG. 1G by double arrow 110). As is further illustrated in FIG. 1G, gigahertz and/or terahertz radiation, which emanates from the scanned body portions and/or objects in response to the illumination, is reflected by the scanning mirror 106, passes through the focusing mirror system 108, is reflected by beam combiner 109 and is detected by detector system 107. A similar system as the system described above is disclosed in the article “Review of Active Millimeter Wave Imaging Techniques for Personnel Security Screening”, written by Zhongmin Wang et al. and published in in the journal “IEEE Access”, vol. 7, pp. 148336-148350. The entire contents of this document is herein incorporated by reference for all purposes. It should be appreciated that the present disclosure is not limited to to such a giga-terahertz imaging system. Specifically, it is conceivable that the giga-terahertz imaging system includes an image sensor. Such an image sensor is described in the article “New Real-Time Sub-Terahertz Security Body Scanner”, written by Gombo Tzydynzhapov et al. and published in the “Journal of Infrared, Millimeter and Terahertz Waves” 41(2) in March 2020. The entire contents of this document is herein incorporated by reference for all purposes.

The above-described giga-terahertz imaging systems are active imaging systems, i.e. imaging systems which include a radiation source 100 which generates radiation having frequencies the gigahertz and/or terahertz frequency range. However, it is also conceivable that the giga-terahertz imaging system is a passive imaging system, i.e. that the imaging system does not include a radiation source but acquires an image of body emitted radiation having frequencies in the gigahertz and/or terahertz frequency range. The radiation, which is emitted by the patient's body may be imaged by an imaging optical system onto an image sensor, which includes a plurality of detector elements, each of which being sensitive to radiation to a plurality of frequencies in the gigahertz and/or terahertz frequency range. The detector elements may be arranged or substantially arranged in an array-like manner. Alternatively, the passive imaging system may include a scanner and an optical system for imaging a portion of the object field onto a single detector element or onto an array of detector elements. The scanner may be configured to successively image portions of the object field in order to image the one or more body portions and/or objects.

It has been shown by the inventors that electromagnetic radiation, which emanates from the patient's body and which is within the terahertz and/or gigahertz wavelength range, traverses sterile drapes or other sheet-like medical covers so that features of the patient's head can be identified with sufficient accuracy for determining the orientation of the laser applicator relative to the patient's head. By way of example, as is illustrated in FIG. 1H, using a giga-terahertz imaging system, it is possible to identify outer contours of body portions of the patient, such as the contour of the skull 111, the chin 112, the neck 113 and the patient's shoulders 114. Giga-terahertz images can also show image features, which relate to the patient's eyes. Since these image features are symmetric about the longitudinal axis LA of the patient's head, it is possible to derive from these features an estimate about the orientation of the longitudinal axis LA within the giga-terahertz image, such as the orientation angle b shown in FIG. 1H. It is also conceivable that the contour of other body portions, such as the contour of the arms 115 and contours of legs 116 are used for determining an estimate of the orientation of the longitudinal axis LA of the head.

It is also conceivable that the determination of the orientation of the laser applicator relative to the patient's head is performed in a similar manner as has been described in connection with the first variant of the exemplary embodiment. Specifically, as has been described with reference to FIG. 1C, using the second imaging device 34 (shown in FIG. 1B), it is possible to align the optical axis of the laser applicator with the axis of the eye to be treated. This allows determination of the orientation of the laser applicator relative to the patient's head by identifying a location in the image acquired using the giga-terahertz imaging system, which corresponds to the location of the untreated eye of the patient.

As is illustrated in FIG. 1I, it is also conceivable that at least portion of the giga-terahertz imaging system (designated with reference numeral 99 in FIG. 1G) is arranged at other portions of the ophthalmic laser system, such as at mounting locations 117 and 118 at the first and second arm segments 8, 11 of the supporting arm 4. Since, depending on the outer shape of the housing of the laser applicator 6, it can be sufficient to determine only a rough estimate of the orientation of the patient's head relative to the laser applicator, it is not necessary that an imaging direction of the orientation analysis system is oriented exactly parallel to the axis of the eye to be treated.

Moreover, it has further been shown by the inventors that the giga-terahertz imaging system can be used for purposes other than determining the orientation of the laser applicator relative to the patient's head. By way of example, as is illustrated in FIG. 1D, since in some surgical procedures, a predominant part of the patient's head is covered by a drape or other medical coverings used in the operating room, e.g. to prevent infections, the giga-and terahertz imaging system can be used for determining the position of the patient's head relative to the laser applicator and/or relative to the base of the ophthalmic laser system. The giga-terahertz imaging system for determining the position of the patient's head can be the same or an additional giga-terahertz imaging system which is provided in addition to the giga-terahertz system, which is used for determining the orientation of the patient's head relative to the laser applicator.

By way of example, a giga-terahertz imaging system, which is mounted at a location 119 (shown in FIG. 1I) at the base can be used for acquiring an image, which is indicative of the height of the patient's head relative to the floor. Therefore, the giga-terahertz imaging system can be used for adjusting the height of the laser applicator and/or the patient's bed. The height of the laser applicator may be adjusted by the motorized three-axis positioning system (designated with reference numeral 9 in FIG. 1I) and/or by moving the positioning arm, either manually or using one or more drive systems.

FIG. 1J is a schematic illustration of a fourth variant of the exemplary embodiment. The orientation analysis system of the fourth variant includes a LIDAR scanning system 119. The LIDAR scanning system includes a laser 120, which forms a laser beam having a spot-shaped or substantially spot-shaped cross-section. The LIDAR scanning system 119 also includes a scanner 121 for scanning the laser beam across a surface, which includes surfaces of the body portions and/or objects. The LIDAR scanning system also includes a detector 122 for detecting light of the laser returned from the surface. The laser 120 may be configured to generate a pulsed laser beam to measure the time of flight (ToF LIDAR), an amplitude-modulated continuous wave laser beam (AMCW LIDAR) or a frequency-modulated continuous wave laser beam (FMCW LIDAR). The scanner 121 may be configured to deflect the laser beam in two dimensions.

The controller of the ophthalmic laser system may be configured to use the orientation-related data which have been acquired using the LIDAR scanning system for generating surface topography data from the one or more body portions and/or objects. The controller may be configured so that based on the surface topography data, the controller identifies the location and/or orientation of one or more anatomical features and/or anatomical landmarks of the patient's body, such as the location and/or orientation of one or both eyes, of one or both ears, of the nose and/or the chin. The controller may also be configured to determine, based on the topography data, a contour of the chin and/or at least a portion of a contour of the head. The contour may relate or substantially relate to a projection on a reference plane of the topography data (i.e. a plane which corresponds to a constant height). Moreover, if the patient's head is covered by a sterile drape, as is illustrated in FIG. 1D, the controller may be configured to identify, automatically or based on user input, a portion of the surface topography data, which corresponds to a nose of the patient. The controller may further be configured to determine, based on the topography data, the orientation of the nose in order to determine the orientation of the laser applicator relative to the patient's head.

Moreover, the controller may be configured to use the height information of the surface topography data to adjust a height of the laser applicator relative to the patient's head.

Additionally or alternatively, the surface topography analysis system may include a 3D time of flight camera system. The 3D time of flight camera system may include a light source and a 3D image sensor. The light source may be configured as a laser and/or a LED. The light source may be a non-scanning light source. The light emitted by the light source may be pulsed, amplitude modulated or may be a stroboscopic light source generating a square wave.

Additionally or alternatively, the surface topography analysis system may include a stereoscopic 3D camera. Using a 3D camera as an orientation analysis system, it is possible to acquiring surface topography data from the one or more objects, which are marked using one or more marks. Specifically, the 3D camera may be sensitive to infrared radiation and the one or more objects may be illuminated using infrared light. This can increase the accuracy in determining the orientation of the laser applicator relative to the patient's head. The controller may use the 3D images which have been acquired using infrared illumination for performing iris registration in order to determine the cyclotorsional angle of the eye to be treated. Also, the controller may use the images for pupil and/or iris tracking.

FIG. 1K is a schematic illustration of an orientation analysis system of a fifth variant of the exemplary ophthalmic laser system. The orientation analysis system of the fifth variant includes an aiming system 101 for aiming at one or more of the body portions and/or objects along an aiming direction, which is schematically illustrated in FIG. 1K by arrow d.

The aiming system 101 includes a light source (not illustrated) for generating an aiming beam 97, which extends along an aiming direction d. In the exemplary embodiment, the aiming beam 97 is a laser beam, in particular a laser beam of a line laser. However, it is also possible that the light source is an incoherent light source. The orientation analysis system may include an optical system, which generates from the light of the incoherent light source an aiming beam, which is rectangular or fan shaped.

As is illustrated in FIG. 1K, the orientation analysis system 101 is configured so that the orientation of the laser applicator 6 relative to the patient's head can be determined without aligning the optical axis OA of the laser applicator 6 with the axis AE of the eye to be treated. Specifically, the aiming beam 97 can be adjusted so that the beam intersects the pupil center of the eye to be treated 29 and the pupil center of the untreated eye 73. The aiming system 101 includes a sensor (not illustrated), which measures the orientation angle g of the aiming direction d relative to the laser applicator about the optical axis OA of the laser applicator 6. By way of example, the light source and focusing optical system of the aiming system 101 is rotatably supported by the laser applicator 6 so that the aiming system is rotatable about the optical axis OA of the laser applicator 6.

After the adjustment of the aiming direction d of the aiming system 101 and the measurement of the orientation g, the laser applicator 6 can be positioned in the plane perpendicular to the axis of the eye to be treated to align the optical axis OA of the laser applicator with the axis of the eye to be treated 29.

Additionally or alternatively, in a similar manner as has been described above in connection with the the first variant, the aiming system 101 of the fifth variant may be configured so that the orientation of the laser applicator relative to the patient's head around the axis of the eye to be treated is adjustable after the optical axis of the laser applicator has been brought into alignment with the axis of the eye to be treated. Specifically, as can be seen from FIG. 1L, after the optical axis OA of the laser applicator has been brought into alignment with the axis of the eye to be treated, the aiming direction the of the aiming system 101 is adjustable so that the aiming beam intersects the pupil center 71 of the untreated eye 73. Since the alignment of the optical axis OA of the laser applicator 6 with the axis of the eye to be treated (using the Purkinje images 86 explained with reference to FIG. 1C) can be performed with a comparatively high accuracy, this results in a more accurate determination of the orientation angle g.

Since the rotation axis (designated with A6 in FIG. 1A) of the laser applicator 6 is displaced relative to the optical axis OA of the laser applicator 6, the adjustment of the orientation of the laser applicator 6 (starting from the configuration shown in FIG. 1L) brings the optical axis OA of the laser applicator 6 out of alignment with the axis of the eye to be treated. However, after the rotation of the laser applicator has been adjusted, it is possible to bring the optical axis of the laser applicator again into alignment with the axis of the eye using the motorized three-axis positioning system (designated with reference numeral 9 in FIG. 1A).

In the above description of the fifth variant, the aiming system is used for aiming at one or both eyes of the patient. However, it is conceivable that the aiming system is configured for aiming at other body portions, such as at the mouth, at the nose or at one or both feet of the patient. By way of example, by aiming at one of the feets of the patient, it is possible to adjust the aiming direction of the aiming system so that it is parallel or substantially parallel to a longitudinal axis of the patient's body. The longitudinal axis of the body indicates or is at least an estimate for the longitudinal axis of the patient's head.

FIG. 1M is a schematic illustration of the orientation analysis system of a sixth variant of the ophthalmic laser system. The orientation analysis system of the sixth variant, includes an aiming system 101, which includes an array of light sources 102, The light sources of the array of light sources 102 are arranged in a circumferential manner on the laser applicator. By way of example, the light sources may be provided by LED light sources. However, the present disclosure is not limited to such light sources. In the sixth variant, the aiming direction d is indicated by an activated light source (designated with reference numeral 103), which points into the aiming direction d. Therefore, by changing the activated light source, the aiming direction d of the aiming system 101 can be changed. The orientation analysis system of the ophthalmic laser system of the sixth variant can be used in the same manner as has been described above with reference to FIGS. 1K and 1L in connection with the fifth variant for determining the orientation angle of the laser applicator 6 relative to the patient's head. Specifically, the determination of the orientation angle can be made with the optical axis of the laser applicator aligned with the axis of the eye to be treated. Alternatively, it is possible do determine the orientation angle without the optical axis of the laser applicator being aligned with the axis of the eye to be treated.

The present disclosure is not limited to aiming systems which use light. As is illustrated in FIG. 1N, a seventh variant of the ophthalmic laser system includes a mechanical indicator 127, which visibly indicates the aiming direction d. By way of example, the mechanical indicator 127 may be a projection, which extends from the housing of the laser applicator. As is illustrated in FIG. 1N, the mechanical indicator 127 of the seventh variant is a projection which is in the form of a tab. However, other shapes of the mechanical indicator 127 are conceivable. In the seventh variant, the mechanical indicator 127 is rotatably supported by the laser applicator 6 so that the aiming direction d of the mechanical indicator 127 is adjustable relative to the laser applicator 6 about the optical axis OA. The aiming system 101 of the seventh variant also includes a sensor (not illustrated), which measures the orientation angle g of the aiming direction d relative to the laser applicator 6 about the optical axis OA of the laser applicator 6.

FIG. 1O is a schematic illustration of an eighth variant of the ophthalmic laser system. The ophthalmic laser system of the eighth variant includes an orientation indication system, which indicates the orientation of the laser applicator 6 relative to the patient's head. The orientation indication system is configured as an aiming system 101. The aiming system 101 is configured to emit an aiming beam 97. However, also other configurations of the aiming system are also conceivable as have been described above in connection with the fifth to seventh variant of the exemplary laser system. The aiming beam may be a laser beam, in particular a laser beam of a line laser. However, it is also conceivable that the light source for generating the aiming beam 97 is an incoherent light source. The orientation analysis system may include an optical system, which generates from the incoherent light a rectangular or fan-shaped light beam. In the exemplary embodiment, which is illustrated in FIG. 1N, the aiming system is configured to emit an aiming beam 97 in a direction d, which has a fixed orientation relative to the laser applicator 6 measured in a plane perpendicular to the axis of the eye to be treated. Therefore, changing an orientation of the laser applicator 6 relative to the patient's head (schematically illustrated by arrow 104) leads to a corresponding change of the aiming direction d of the aiming beam 97 (schematically illustrated by double arrow 123. Additionally or alternatively, the orientation indicating system may include a mechanical indicator, which may be a projection, which extends from the housing of the laser applicator. The mechanical indicator may be configured as a projection. The projection may be in the form of a tab. However, other shapes of the mechanical indicator are also conceivable. The mechanical indicator may have a fixed orientation relative to the laser applicator measured in a plane perpendicular to the axis of the eye to be treated.

In order to bring the laser applicator into a desired orientation relative to the patient's head, measured about the axis of the eye to be treated, the user and/or controller can rotates the laser applicator 6 until the beam 97 of the aiming system intersects the pupil centers of both eyes. After the adjustment of the rotation of the laser applicator 6, the user and/or controller can adjust the position of the laser applicator 6 relative to the patient's hand using the motorized three-axis positioning system (designated with reference numeral 9 in FIG. 1A).

The ophthalmic laser systems according to any one of the preceding variants 1 to 7 may be configured so that the controller uses the determined one or more parameters of the orientation of the laser applicator 6 relative to the patient's head for determining the positioning data, which are indicative of correlated with the locations of the laser focus within the eye to be treated. The positioning data may include parameters of one or more scanning paths for performing the laser treatment.

By way of example, the controller may use a determined rotation angle of the laser applicator relative to the patient's head for determining the angular location of arcuate keratotomy incisions, limbal relaxing incisions, phaco incisions and/or orientations a flap hinges, measured about the axis of the eye to be treated.

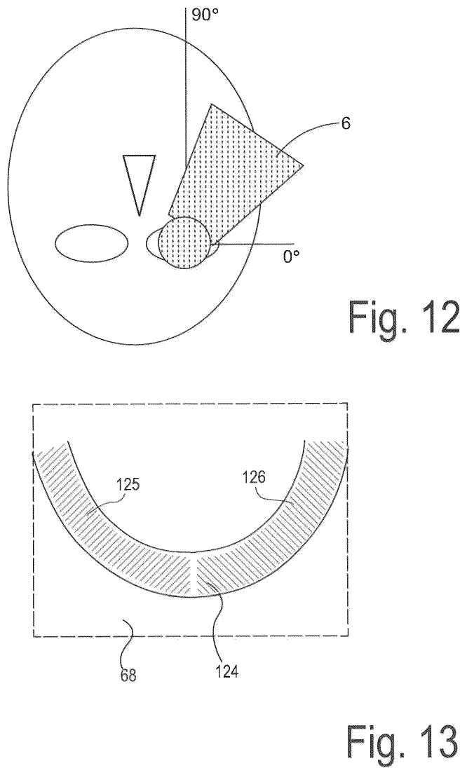

Additionally or alternatively, in the exemplary laser system of any one of the preceding variants 1 to 7, the controller of the ophthalmic laser system may be configured to determine, based on the orientation-related data, whether an exit optical element of the laser applicator is positioned in front of the left or in front of the right eye of the patient. Thereby, the controller can verify that the laser applicator is positioned in front of the eye, which is intended to be treated. By way of example, for the ophthalmic laser system of the fist variant, which is illustrated in FIG. 1C, due to the outer shape of the housing of the laser applicator, in the position, which is represented by the images, which are shown in FIG. 1C (i.e. in a position of the laser applicator in which the image of the first imaging system is acquired), the range of possible orientations of the laser applicator relative to the patient's head is limited to an angular range of approximately 90 degrees (see FIG. 12). As is schematically illustrated in FIG. 13, there is an image region 124 within which the untreated eye of the patient appears in the image of the first imaging system. Due to the above-mentioned limited angular range for the orientation of the laser applicator 6, region 124 can be separated into two non-overlapping regions 125 and 126. If the image portion, which corresponds to the untreated eye appears in region 125, the eye, which is imaged by the second imaging system 34 (shown in FIG. 1B), is the right eye of the patient. On the other hand, if the image portion, which corresponds to the untreated eye appears in region 126, the eye, which is imaged by the second imaging system 34 is the left eye of the patient.

The laser system according to the exemplary embodiment includes a braking and/or locking system for arresting a movement of the second end of the supporting arm relative to the first end of the supporting arm. Specifically, this allows the surgeon to perform the coarse positioning procedure by manually positioning the laser applicator 6 into a position close to the patient's head. After the course positioning procedure, the surgeon activates a braking and/or locking system of the supporting arm so that in the arrested state, the surgeon can perform the fine positioning procedure using the positioning system 9.

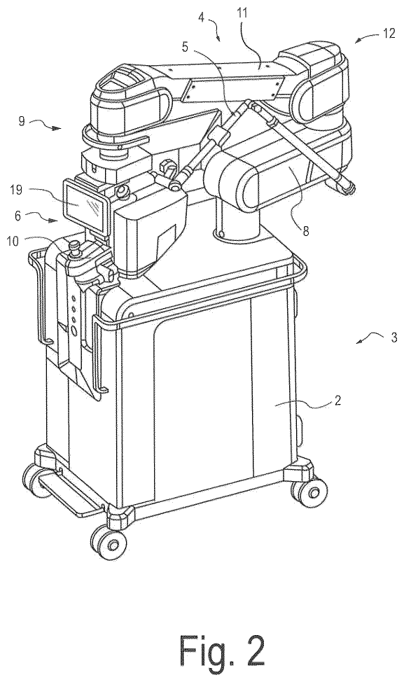

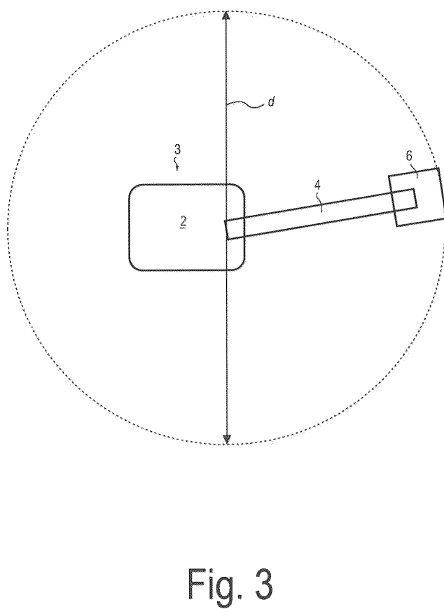

In the exemplary embodiment, the laser applicator 6 includes one or more manually operable control elements 18a, 18b (shown in FIG. 4), which allow the surgeon to deactivate the braking and/or locking system so that the applicator head can be positioned in to a different position using the supporting arm 4. In the exemplary embodiment, the laser applicator includes two handles 17a, 17b (shown in FIG. 4), which can be grasped by both hands of the surgeon. At each of the handles a release button 18a, 18b is provided. If the surgeon simultaneously presses both release buttons, the braking and/or locking system is deactivated and the surgeon can position the laser applicator 6 in three dimensions. For simplicity of illustration, in FIGS. 1A and 2, the handles 17a, 17b and the release buttons 18a and 18b are not shown.

The laser system according to the exemplary embodiment is configured to allow positioning the laser applicator relative to the patient's head by performing a coarse positioning procedure in which the laser applicator 6 is adjusted relative to the patient's head using the supporting arm 4. Then, in a subsequent fine positioning procedure using the three-axis positioning system 9, the laser applicator 6 is positioned to its final position relative to the patient's head in which the laser treatment is carried out.

The coarse positioning procedure using the supporting arm 4 allows the surgeon to perform a fast and efficient coarse positioning relative to the patient's head. The manual adjustability also provides increased safety for the patient, since the surgeon can rapidly move the laser applicator 6 to a location distant from the patient's head if needed. However, it is conceivable that one or more joints of the supporting arm are motorized so that the course positioning is performed fully or partially (i.e. using manual positioning) using the motor.

As is explained in more detail further below, the fine positioning procedure may be performed based on images of an imaging system, which is part of the laser applicator 6 and/or based on measurement values of an interaction measurement unit, which measures a mechanical interaction, such as a force, between the patient's eye and the laser applicator 6.

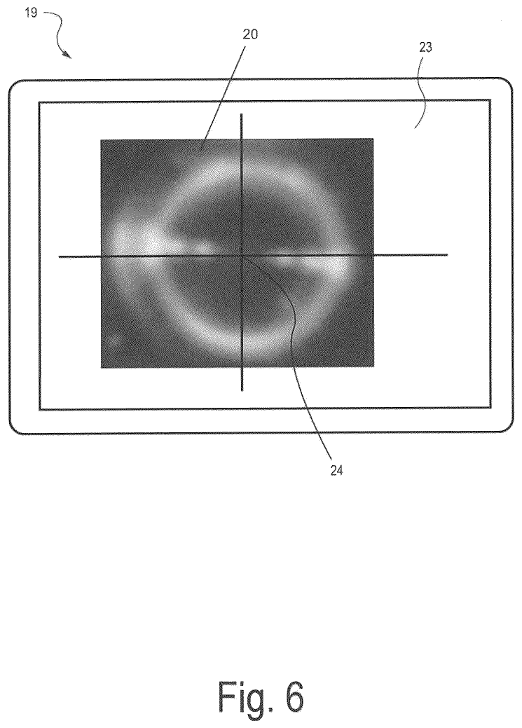

FIG. 6 schematically illustrates information, which is displayed on a display device 19 of the laser applicator 6 during the coarse positioning procedure. As can be seen from FIG. 6, the surgeon sees an image on the display device 19, which is generated by an imaging system, which is provided within the laser applicator 6. The imaging system acquires a frontal image 20 of the patient's eye using a constant object plane distance. The object plane distance is adjusted so that it corresponds to a desired pre-defined distance between the laser applicator and the patient's eye. Therefore, using the frontal image of the eye, the surgeon can control a lateral position of the laser applicator using the three-axis positioning system so that the center of the crosshair 24 lies in the center of the pupil of the eye. Further, by adjusting a vertical position of the laser applicator 6 using the positioning system 9 until an in focus image appears on the display device 19, the surgeon can adjust the height of the laser applicator so that the distance between the laser applicator 6 and the patient's eye corresponds to the desired pre-defined distance.

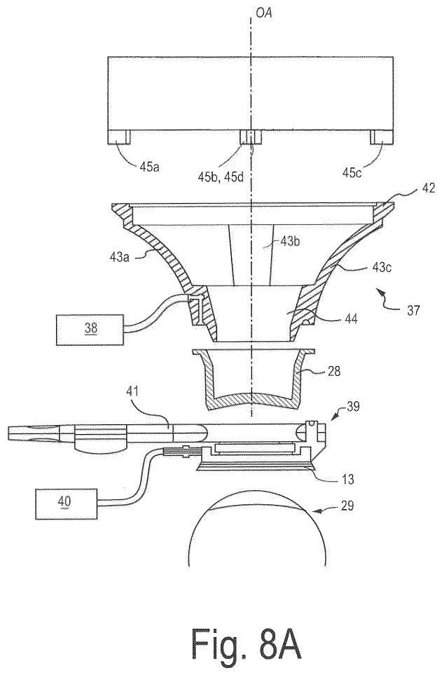

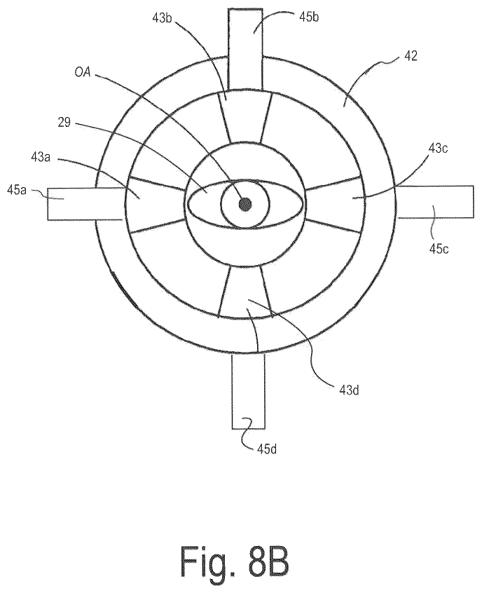

The above course positioning procedure may be carried out in a state in which the suction ring and the contact element (described below with reference to FIGS. 7 and 8A) are attached to the eye. The laser applicator 6 and the contact element may be configured so that the iris and the limbus are displayed in the frontal image 20. By way of example, the surgeon determines based on a portion or all of these features, whether or not the frontal image 20 is in focus.

The controller of the laser system may include an image processing algorithm for determining, whether at least a portion of the frontal image 20 is in focus and/or which is configured to determine one or more parameters, which depend on, or which are indicative of, a level of focus of at least a portion of the frontal image 20. By way of example, the portion of the frontal image 20 may be the iris of the eye. The image processing algorithm may include a segmentation algorithm for segmenting the frontal image. The image processing algorithm may determine one or more parameters, which depend on, or which are indicative of, a level of focus of one or more of the segmented image regions. By way of example, a segmented image region may represent the iris of the patient's eye.

The controller may be configured to display on the display device 19 graphical and/or textual information based on the determined parameters.

The arrangement of the imaging system within the laser applicator 6 is described in the following with reference to FIG. 7. The laser applicator 6 includes a beam combiner 26. The beam combiner 26 is in the beam path of the treatment laser beam 27 between a scanning system (not shown in FIG. 7) and a contact element 28 of the patient interface. The contact element 28 includes a concave contact surface, which contacts the anterior surface of the cornea during the treatment. It is noted that the concave shape of the contact element 28, which is shown in FIG. 7 is only an example and it is conceivable that the contact surface of the contact element 28 is planar or convex toward the eye.

The beam combiner 26 may be in the beam path of the treatment laser beam 27 between two components 30a and 30b of the focusing optical system 36, as it is illustrated in FIG. 7. The focusing optical system 36 is also arranged within the laser applicator 6. It is also conceivable that the laser applicator includes at least a portion of a scanning system for three-dimensionally scan a focus of the treatment laser beam within the eye. The scanning system may include an axial scanning system for scanning the laser focus along an axis of the laser beam and/or a beam deflection scanning system for scanning the laser beam through deflection of the laser beam.

Each of the components 30a and 30b may include one or more optical elements, such as lenses. However, the present disclosure is not limited to such a configuration. It is also conceivable that the beam combiner 26 is either in the beam path of the treatment laser beam 27 between the scanning system and the focusing optical system or in the beam path of the treatment laser beam 27 between the focusing optical system and the contact element 28.

The beam combiner 26 may include a semi-transparent mirror and/or a prism. The semi-transparent mirror may be a dichroic mirror and/or the prism may be dichroic prism. As it is schematically illustrated in FIG. 7, the beam combiner 26 may be configured to combine the beam path of the laser beam 27 on the one hand with a measurement beam path 31 of an optical coherence tomography (OCT) system 32 and an imaging beam path 33 of the imaging system 34 on the other hand. The imaging system may have a two-dimensional light sensitive imaging sensor. The light sensitive image sensor may have a two-dimensional array of light sensitive pixels. The optical coherence imaging system may be configured to acquire cross-sectional images of the cornea and/or the natural lens of the eye. The imaging system, which has the imaging sensor may be configured to acquire the two-dimensional frontal image of the eye.

In the eye treatment system according to the exemplary embodiment, the measurement beam path 31 of the optical coherence system 32 and the imaging beam path 33 of the imaging system 34 are combined using a second beam combiner 35, which is outside the beam path of the treatment laser beam 27. The second beam combiner 35 may include a mirror and/or a prism. The mirror may be a dichroic mirror and/or the prism may be a dichroic prism.

The cross-sectional images of the OCT system 32 can be used during the fine positioning procedure for observing, whether or not the anterior surface of the cornea has contacted the contact element 28.

As is discussed in the following with reference to FIG. 8, during the fine positioning procedure, the position of the laser applicator relative to the patient's eye is monitored based on signals of an interaction measuring unit, which generates an output signal, which depends on a mechanical interaction between the patient's eye and the laser applicator.

FIG. 8A schematically illustrates - in an exploded view - the contact element 28 and further components, which are used for coupling the contact element 28 relative to the laser optical system on the one hand and relative to the patient's eye 29 on the other hand. The laser system includes a coupling portion 37, which may be in rigid connection with the laser optical system or which may be displaceably supported in a direction parallel to the optical axis of the laser optical system. The contact element 28 and the coupling portion 37 are configured so that the contact element 28 is detachably coupleable to the coupling portion 37. In the coupled state, the contact element 28 may be in a substantially predefined position relative to the laser optical system and may have a pre-defined inclination relative to the optical axis OA of the laser optical system. Alternatively, in the embodiment, in which the contact element 28 is displaceably supported in a direction parallel to the optical axis of the laser optical system, in the coupled state, the contact element 28 is in a pre-defined radial position relative to the optical axis and has a pre-defined inclination relative to the optical axis. The contact element 28 is attached to the coupling portion 37 using a suction mechanism, which includes a suction source 38.

The laser system further includes a suction ring 39 which can be secured to the eye 29 and to which the contact element 28 is rigidly attachable. The suction ring 39 includes a skirt that forms a groove, which defines a suction channel between the skirt and an anterior surface of the eye 29. Generation of a vacuum in the vacuum passage using a vacuum source 40 therefore fixedly attaches the the suction ring 39 to the anterior surface of the eye 29.

The suction ring 39 is rigidly attached to a clamp mechanism 41 or formed with the clamp mechanism 41 as a single piece. The clamp mechanism 41 is used for securing the contact element 28 to the suction ring 39. One example for such a clamp mechanism 41 is disclosed in document US 2007/0093795 A1, the contents of which is incorporated herein by reference for all purposes. However, the present invention is not limited to configurations in which the contact element 28 is secured to the suction ring 39 using a clamp mechanism. Specifically, it is conceivable that the contact element 28 and the suction ring 39 are integrally formed, such as formed as a single piece or integrated into a one-piece assembly.

As can be seen from FIG. 8A and also from FIG. 8B, which is a top view of the coupling portion 37 and the eye 29, the coupling portion 37 includes a coupling ring 42 for coupling the coupling portion 37 to the remaining portion of the laser applicator. Further, the coupling portion 37 includes a plurality of extension arms, 43a, 43b, 43c, 43d, each of which connecting the coupling ring 42 to a lower portion 44 of the coupling portion 37.

When the coupling portion 37 mounted to the remaining portion of the laser applicator, the coupling portion 37 contacts a plurality of force sensors 45a, 45b, 45c, 45d. The force sensors 45a, 45b, 45c, 45d are arranged in a plane which is perpendicular to the optical axis OA of the laser applicator and therefore parallel to the plane of the mounting ring 42. As can be seen from FIG. 8B, the force sensors are circumferentially distributed at equal angles about the optical axis OA of the laser applicator and also at a same distance from the optical axis OA. By way of example, each of the force sensors 45a, 45b, 45c and 45d include piezoelectric force sensor, which measures a tensile and/or compressive force acting on the piezoelectric force sensor. Additionally or alternatively, strain gauges are arranged on one or more of the extension arms.

As can be seen from the cross-sectional illustration of FIG. 8A, each of the force sensors 45a, 45b, 45c and 45d is arranged in a force path between the patient's eye 29 and the focusing optical system 36 (shown in FIG. 7), which focuses the treatment laser beam within the patient's eye 29 and which is part of the laser applicator. Therefore, a force magnitude, which is measured by each of the respective force sensor, as well as differences between force magnitudes measured by different force sensors can be used for determining the magnitude and direction of the force between the laser applicator and the eye.

Therefore, monitoring the output signals of the force sensors 45a, 45b, 45c and 45d allows docking the laser system to the eye with circumferentially uniform forces which ensure that the eye is not tilted during the laser treatment. Uniform forces measured by the force sensors are particularly important in capsulotomy and lens fragmentation procedures, where a “soft docking” technique is used.

For performing the “soft docking” technique, contact element 28 (shown in FIG. 8A) is used, which has a concave contact surface for contacting the anterior surface of the cornea. The forces between the patient's eye and the laser applicator are kept at a low level so that in the docked state, a thin layer of saline solution is present between the contact surface of the contact element and the anterior surface of the cornea of the eye.

Using the output signals of the force sensors for performing the “soft docking” techniques ensures that the vertical force component does not exceed a pre-defined level so that there are only minimal corneal distortions and posterior corneal folds are avoided. Posterior corneal folds can deflect the treatment laser beam which can result in “postage stamp-like” incisions. Further, by ensuring that a lateral component of the force does not exceed a pre-defined level, it is ensured that the eye is not tilted, which increases the accuracy of the laser treatment.

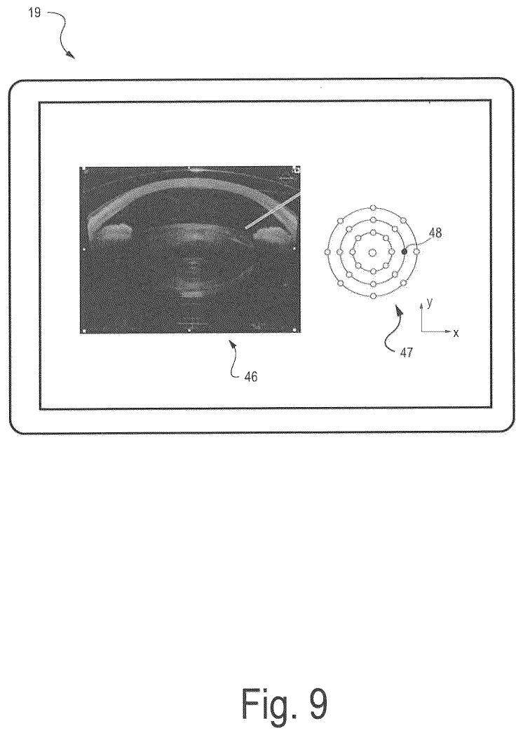

FIG. 9 is a schematic illustration of the information, which is displayed on the display device 19 during the fine positioning procedure. The display device shows an OCT image 46, which is acquired during the docking procedure, as well as a diagram 47, which is generated based on the output signals of the force sensors 45a, 45b, 45c and 45d (shown in FIGS. 8A and 8B). The OCT image may be a real-time OCT image.

In the exemplary embodiment, the diagram 47 has three concentric rings, wherein an indicator is arranged in the center and each of the rings has eight indicators. After processing of the output signals of the interaction measuring sensors by the controller, one of the indicator is highlighted, such as indicator 48 in FIG. 9. The ring on which the highlighted indicator is located indicates the magnitude of the measured force, wherein a greater diameter of the ring indicate a higher force magnitude measured by the force sensors. Specifically, the inner two rings indicate an acceptable force level, wherein the outer ring indicates an unacceptable force level. If the force level is unacceptable, the surgeon uses the positioning system 9 (shown in FIG. 1A) which is controllable using the control unit 10 to move the laser applicator in a direction away from the patient by raising the laser applicator. Adjusting the laser applicator so that the vertical force level is below a pre-defined level ensures that corneal folds are avoided.

The circumferential position of the highlighted indicator indicates a direction of a lateral component of the force measured using the force sensors. In FIG. 9, the highlighted indicator is located on the right side, which indicates to the surgeon that the laser applicator should be moved in the negative x direction in order to minimize lateral forces. If the lateral forces are minimized this avoids that the eye is tilted relative to the optical axis of the laser applicator.

Additionally or alternatively, the controller may be configured to determine a parameter, which is indicative of, or which depends on, a magnitude of at least a component of the force between the laser applicator and the patient's eye. The controller may be configured to display on the display device the parameter. The component of the force may be a component along an optical axis of the laser applicator or a component in a plane, which is oriented perpendicular to the optical axis of the laser applicator.

Therefore, using the force sensors and the three-axis positioning system allows the surgeon to perform a docking procedure which ensures a high quality of the surgical procedure.

In the laser system according to the exemplary embodiment, the output signals of the interaction measuring unit are used to determine, whether to deactivate the braking and/or locking system based on the output signals of the interaction measuring unit. Thereby, it is possible to prevent injuries to the patient's eye in the event that the patient moves his head during the laser treatment. By way of example, if a projection of the force vector onto the optical axis of the laser applicator exceeds a pre-determined threshold, the laser source and the braking and/or locking system is deactivated.

FIG. 10 is a cross-sectional view of the parallel linkage, which includes the second arm segment 11 in the laser system according to the exemplary embodiment, which is shown in FIG. 1A. The second arm segment 11 comprises two bars 52 and 53 of a four bar linkage, which is configured as a parallel linkage having the bars 52 and 53 of the second arm segment, which are oriented parallel relative to each other and four joints 53a, 53b, 53c and 53d, each of which having a horizontal rotation axis.

The parallel linkage also includes a counterbalancing mechanism for providing a counterbalancing that at least partially counteracts a gravitational force G acting on the laser applicator (not shown in FIG. 10). In the exemplary embodiment, the counterbalancing mechanism includes a compression spring 54, which is arranged within the first bar 53 and which exerts a pulling force on a toothed belt 55, which extends into the second bar 52 via two toothed belt wheels 56, 57. Within the second bar 52, an end of the toothed belt 55 is fastened using a fastening member 58.

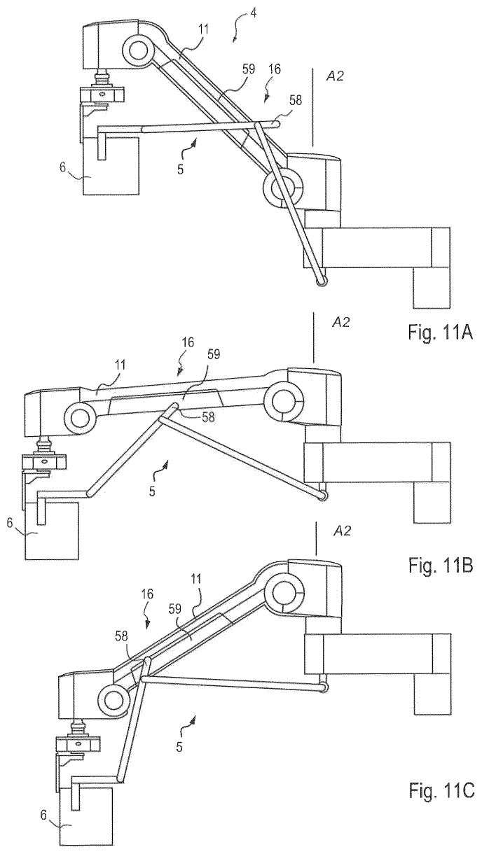

Returning to FIG. 1A, the laser system according to the exemplary embodiment includes a coupling mechanism for coupling the articulated beam guide tube 5 to the second arm segment 11 of the supporting arm 4. This coupling mechanism 16 is illustrated in more detail in the side views of FIGS. 11A to 11C.

As can be seen from FIGS. 11A to 11C, the coupling mechanism 16 includes a first coupling member 58, which is rigidly attached or integrally connected to the articulated beam guide tube 5. Furthermore, the coupling mechanism includes a second coupling member 59, which is a corresponding coupling member to the first coupling member 58 and which is attached or integrally connected to the second arm segment 11. In the exemplary embodiment, the first and second coupling members 58, 59 are configured as a lateral guide wich limits a lateral movement of the first coupling member 59 relative to a direction, which is parallel to a longitudinal axis of the second arm segment 11. In the exemplary embodiment, the coupling mechanism 16 has the effect that a plane, which is defined by the neighboring tube segments 13 and 14 of the articulate beam guide tube 5 has a substantially vertical orientation, for different positions of the laser applicator 6, which are assumed through movements of the first and second arm segments 8 and 11 of the supporting arm 4. This ensures that during operation of the laser system 1, the articulated beam guide tube 5 does not collide with with the supporting arm 4. Such a collision can damage the articulated beam guide tube 5 and/or the supporting arm 4 or can block the positioning of the laser applicator 6 using the supporting arm 4.

Specifically, FIGS. 11A to 11C schematically illustrate for each of three different configurations of the supporting arm 4 how the fist and second coupling members 58, 59 are positioned relative to each other. For different inclinations of the second arm segment 11, the joint between the first and second arm segment 13 and 14 of the articulated beam guide tube 5 remains coupled to the second arm segment 11 of the supporting arm 1. Further, since the articulated beam guide tube extends between the laser applicator 6 and a location, where the treatment laser beam exits from the first arm segment 8 of the supporting arm 4, the articulated beam guide tube 5 can remain engaged with the linear guide even if the second arm segment is rotated about the vertical axis A2. However, it is noted that if the lateral guide has a sufficient play, the articulated beam guide tube 5 can also be coupled to the second arm segment 11 even if the articulated beam guide tube extends from a location, where the treatment laser beam exits from the base 3.

Additionally or alternatively, it is also conceivable that the coupling arrangement includes a tensile force transmitting connection.

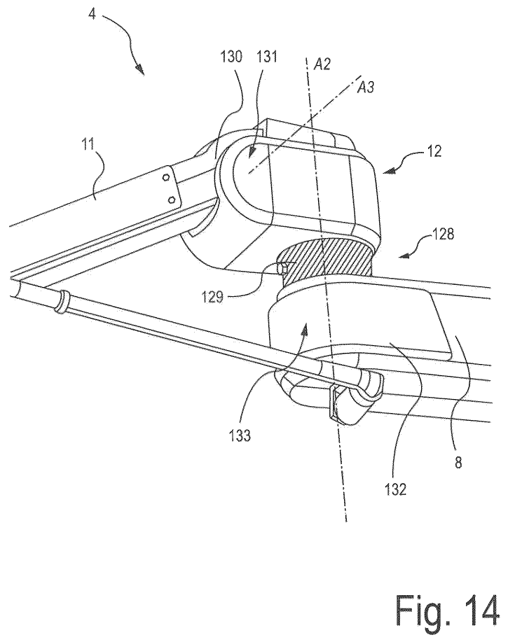

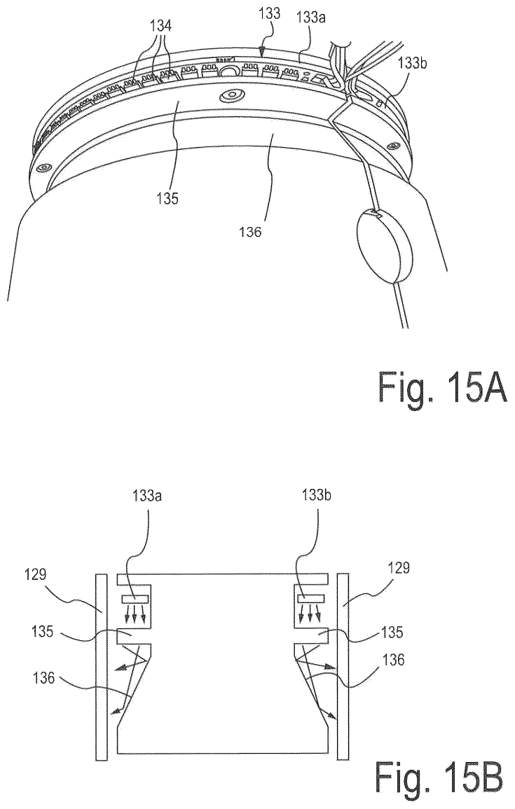

FIG. 14 is a schematic illustration of a portion of the supporting arm 4 of the ophthalmic laser system of the exemplary embodiment, which incudes a controllable visual indicator 128, which is in operative connection with the controller (not shown in FIG. 15) of the laser system. The controller controls the visual indicator 128 to generate a visual signal. The visual signal may assist the surgeon to perform the laser surgical treatment and/or indicate a warning signal in order to ensure a successful laser treatment.

As has been discussed above with reference to FIGS. 1A, 4 and 5, the supporting arm includes a first arm segment 8 which is connected to a second arm segment 11 via an intermediate joint 12. The intermediate joint 12 therefore represents a supporting structure for the second arm segment 11. The intermediate joint 12 may be configured so that an orientation of the second arm segment 11 relative to the first arm segment is adjustable in two rotary degrees of freedom. In particular, the intermediate joint 12 may be configured so that the second arm segment 11 is rotatable about the vertical axis A2 and also rotatable about the horizontal axis A3. Specifically, as can be seen from FIG. 14, a proximal portion 130 of the second arm segment 11 forms part of a first rotary joint 131 of the intermediate joint 12, wherein the axis of the first rotary joint 131 is the horizontal axis A3. Moreover, a distal portion 132 of the first arm segment 8 forms part of a second rotary joint 133 of the intermediate joint 12, wherein the axis of the second rotary joint 133 is the vertical axis A2.

It should be appreciated that the present disclosure is not limited to visual indicators, which are arranged at an intermediate joint of the supporting arm. It is also conceivable that the visual indicator is mounted to or is part of an arm segment of the supporting arm.

The intermediate joint 12 includes the visual indicator 128. The visual indicator 128 includes a curved surface 129, from which visual signal light, which is generated by the visual indicator 128, emanates. The curved surface 129 forms an outer circumferential surface of the supporting arm. Therefore, in the exemplary embodiment, the curved surface fully surrounds a portion of the supporting arm 4. However, it is also conceivable that the curved surface from which the visual signal light emanates, only forms a circumferential portion of an outer circumferential surface of the supporting arm. By way of example, the circumferential portion may represent more than 50%, more than 70% or more than 80% of the circumference of the circumferential surface.

Since the visual signal light emanates from a curved surface, which forms a circumferential portion of the the supporting arm, the visual signal is easily visible from different viewing directions. This ensures that the visual signal is visible to all surgeons and surgical staff members, who are present in the operating room.

The curved surface 129 from which the visual signal light emanates may be formed from a cylindrical or substantially cylindrical cover, which covers one or more light sources of the visual indicator. The cover may be made from transparent, in particular from light diffusing material.

Additionally or alternatively, the visual indicator may include a plurality of surfaces, wherein from each of the surfaces, visual signal light emanates. The plurality of surfaces are distributed at least around a circumferential portion of the outer circumferential surface of the supporting arm. By way of example, each of the surfaces may be a surface of a light source, such as an LED light source.

As can further be seen from FIG. 14, an axis of the circumferential portion, is oriented or substantially oriented in a vertical direction. Therefore, the visual signal can be seen from different viewing directions within the operating room.

The controller may be configured so that the visual signal indicates a stage of the laser treatment. The stage of the laser treatment may be indicated in a pre-determined time correlation or in time synchronization with a progress of the laser treatment. By way of example, the visual signal may indicate to the user a progress of the laser treatment. By way of example, the indicated progress may be be indicative of or correlated with a percentage value between 0% and 100%. By way of example, the controller may be configured so that a frequency of an intensity variation of the visual signal and/or a color of the visual signal is indicative of or correlated with the percentage value.

Additionally or alternatively, the controller may be configured so that the visual signal indicates an operational state of the of ophthalmic laser system. The controller may be configured so that the operational state of the ophthalmic laser system is indicated in time correlation or in time synchronization with changes in the operational state of the ophthalmic laser system. Examples for operational states of the treatment laser which are indicated to the user are but are not limited to: “laser standby”, “laser ready”, “laser emission”. Each of these states may correspond to a pre-defined visual signal of the visual indicator, which, for example, is defined by a frequency of an intensity variation of the visual signal and/or a color of the visual signal.

Additionally or alternatively, the controller and the visual indicator may be configured so that the visual indicator indicates a warning message to a user. Examples for warning messages are but are not limited to: “laser beam energy too low”, “laser beam energy too high”, “force out of range”. The force may be measured using the interaction measuring unit described above with reference to FIG. 9. Each of these warning messages may correspond to a pre-defined visual signal of the visual indicator, which, for example, is defined by a frequency of an intensity variation of the visual signal and/or a color of the visual signal.

FIG. 15A is a schematic perspective illustration and FIG. 15B is a schematic cross-sectional illustration of a portion of the intermediate joint 12 (also shown in FIG. 14). In FIG. 15A, the portion of the intermediate joint is shown in a state in which the curved surface (designated in FIGS. 14 and 15B with reference numeral 129) is removed from the intermediate joint 12. In the laser system of the exemplary embodiment, the curved surface 129 is a cover, which covers one or more light sources 134, which may be configured as light emitting diodes (LEDs). The cover also covers a light-diffusing sheet 135 and a reflector 136, which is configured for specular or diffuse reflection of signal light, which is emitted from the one or more light sources 134. The light diffusing sheet 135 is made from a light diffusing material, such as a light diffusing synthetic plastic material. At least a portion of the cover 129 may be made from light diffusing material and/or from a material which is transparent for one or more wavelengths of the light, which is emitted from the one or more light sources 134. In the exemplary embodiment of the ophthalmic laser system, the one or more light sources 134 are mounted on a carrier structure 133, which, in the exemplary embodiment, is configured as a circuit board. In the ophthalmic laser system of the exemplary embodiment, the carrier structure 133 is formed from two boards 133a, 133b, each being in the shape of a half ring having a plane or substantially plane shape. However, it is also conceivable that the carrier structure 133 is formed from one board, which only partially surrounds the circumference. Alternatively, it is also conceivable that the carrier structure is formed from three or more boards, wherein the three or more boards together fully or partially surround the circumference. Signal light, which is emitted from the one or more light sources 134, traverses the light-diffusing sheet 135, is reflected by the reflector 136 by diffuse or specular reflection and then traverses the cover 129. Thereby, it is possible to provide a comparatively large cover from which signal light emantes so that the visual signal is sufficiently clearly visible to all surgeons and medical staff members in the operating room.

The above embodiments as described are only illustrative, and not intended to limit the technique approaches of the present invention. Although the present invention is described in details referring to the preferable embodiments, those skilled in the art will understand that the technique approaches of the present invention can be modified or equally displaced without departing from the protective scope of the claims of the present invention. In the claims, the word “comprising” does not exclude other elements or steps, and the indefinite article “a” or “an” does not exclude a plurality. Any reference signs in the claims should not be construed as limiting the scope.

Before we go on to set out the claims, we first set out the following clauses describing some prominent features of certain embodiments of the present disclosure.

Clause 1: An ophthalmic laser system configured to perform laser treatments on an eye of a patient using a treatment laser beam, the laser system comprising: a beam delivery system for focusing the treatment laser beam so that the treatment laser beam travels toward the eye to be treated; a controller operatively coupled with the beam delivery system and configured to control the beam delivery system to position a focus of the laser beam at different locations within the eye or on an anterior surface of the cornea of the eye; a guide configured to allow moving at least a portion of the beam delivery system, to adjust an orientation of at least the movable portion to different orientations of the patient's head measured around an axis of the eye to be treated; an orientation analysis system for acquiring orientation-related data using (a) one or more body portions of the patient; and/or (b) one or more objects, each of which having a fixed position and/or orientation relative to a respective body portion so that the orientation-related data depend on an orientation of the one or more body portions around the axis of the eye; wherein the ophthalmic laser system is configured to allow a user and/or the controller, using the orientation-related data, to move the movable portion of the beam delivery system so that during the laser treatment, the movable portion has an orientation around the axis of the eye and relative to the patient's head, which (i) corresponds or substantially corresponds to a pre-determined target orientation; or which (ii) is within or substantially within a pre-determined target range.

Clause 2: The ophthalmic laser system of clause 1, wherein at least one of the body portions is outside the eyeball of the eye to be treated.

Clause 3: The ophthalmic laser system of clause 1 or 2, wherein the guide includes a rotational guide configured to allow rotation of the movable portion of the beam delivery system about a rotation axis.

Clause 4: The ophthalmic laser system of clause 3, wherein the laser system is configured so that during laser treatment, the rotation axis of the rotational guide is parallel to or aligned with an optical axis of the movable portion of the beam delivery system.

Clause 5: The ophthalmic laser system of any one of the preceding clauses, wherein the controller is configured to determine, automatically or using user input, based on the orientation-related data, one or more parameters, which are (a) indicative of or (b) correlated with one or more, or all, parameters of an orientation of the patient's head around the axis of the eye to be treated.

Clause 6: An ophthalmic laser system configured to perform laser treatments on an eye of a patient using a treatment laser beam, the laser system comprising: a beam delivery system for focusing the treatment laser beam so that the treatment laser beam travels toward the eye to be treated; a controller operatively coupled with the beam delivery system and configured to control the beam delivery system to position a focus of the laser beam at different locations within the eye or on an anterior surface of the cornea of the eye based on laser positioning data, which are indicative of or correlated with the locations within the eye to be treated; an orientation analysis system for acquiring orientation-related data using (a) one or more body portions of the patient and/or (b) one or more objects, each of which being in a fixed position and/or orientation relative to a respective body portion, so that the orientation-related data depend on an orientation of the one or more body portions around the axis of the eye; wherein at least one of the body portions is outside of an eyeball of the eye to be treated; wherein the controller is further configured to determine, based on the orientation-related data: (i) one or more parameters of the laser positioning data; and/or (ii) whether an exit optical element of the beam delivery system is positioned in front of the left or in front of the right eye of the patient.

Clause 7: The ophthalmic laser system of clause 6, wherein the controller is configured to determine, based on the orientation-related data, automatically or using user input, one or more parameters, which are indicative of or correlated with one or more, or all, parameters of an orientation of the patient's head.

Clause 8: The ophthalmic laser system of clause 7, wherein (a) the one or more parameters of the orientation of the patient's head are measured around the axis of the eye to be treated; and/or (b) the one or more parameters of the orientation of the patient's head are measured (i) relative to the movable portion of the beam delivery system; or (ii) relative to a stationary coordinate system.

Clause 9: The ophthalmic laser system of clauses 5, 7 or 8, wherein the determination of the one or more parameters comprises: (a) determining, automatically or based on user input, one or more or all parameters of an angular position of an anatomical feature of the patient and/or an anatomical landmark of the patient relative to the axis of the eye; and/or (b) determining, automatically or based on user input, one or more or all parameters of an orientation of the anatomical features and/or the anatomical landmark in a plane perpendicular to the axis of the eye.

Clause 10: The ophthalmic laser system of any one of the preceding clauses, wherein the orientation analysis system comprises a detector system, which is configured to detect electromagnetic radiation emanating from a region that comprises one or more of the body portions and/or objects.

Clause 11: The ophthalmic laser system of clause 10, wherein the detector system is configured to detect the electromagnetic radiation in a spatially resolved manner.

Clause 12: The ophthalmic laser system of clause 10 or 11, wherein the electromagnetic radiation has frequencies, which are within a range of between 10 GHz and 800 THz and/or has wavelengths, which are within a range of between 10 micrometers and 10 millimeters, in particular within a range of between 100 micrometers and 1 millimeter.

Clause 13: The ophthalmic laser system of any one of the preceding clauses, wherein the orientation data are acquired using the one or more objects; wherein the orientation analysis system is configured to acquire the orientation-related data using a magnetostatic and/or electrostatic field extending between a portion of the ophthalmic laser system, in particular between the movable portion of the beam delivery system and the one or more objects.

Clause 14: The ophthalmic laser system of any one of the preceding clauses, wherein the orientation analysis system comprises: (a) a 2D imaging system for acquiring a two-dimensional image of the one or more body portions and/or objects; and/or (b) a surface topography acquisition system; and/or (c) a 3D imaging system.

Clause 15: The ophthalmic laser system of any one of the preceding clauses, wherein the ophthalmic laser system comprises one or more inclinometers and/or accelerometers, wherein the controller is configured to reduce or eliminate degradation of the orientation-related data which is caused by a movement of the movable portion of the beam delivery system relative to the patient's head.

Clause 16: The ophthalmic laser system of any one of the preceding clauses, wherein an optical path of the orientation analysis system traverses (a) one or more optical elements of the beam delivery system and/or (b) an optical element, in particular a patient interface, which is arranged in a beam path of the treatment laser beam between the beam delivery system and the eye to be treated.

Clause 17: The ophthalmic laser system of any one of the preceding clauses, wherein the orientation-related data comprise a pictorial, symbolic and/or topographic representation and/or a 3D image, which comprises one or more of the body portions and/or objects.

Clause 18: The ophthalmic laser system of clause 17, wherein the controller is configured to determine, automatically or using user input, a position and/or an orientation of the one or more body portions and/or objects in the representation and/or 3D images.

Clause 19: The ophthalmic laser system of any one of the preceding clauses, wherein the laser system is configured to display on a display device a pictorial, symbolic and/or topographic representation and/or a 3D image which is indicative of a position and/or an orientation of the movable portion of the beam delivery system relative to a body portion of the patient, in particular relative to the patient's head.

Clause 20: The ophthalmic laser system of any one of the preceding clauses, wherein the laser system is configured to determine, based on the orientation-related data, one or more parameters of a positioning movement of the movable portion of the beam delivery system.

Clause 21: The ophthalmic laser system of any one of the preceding clauses, wherein at least one of the body portions comprise at least a portion of the patient's head, in particular (a) at least a portion of the eyeball of the untreated eye and/or (b) at least a portion of the eyeball of the eye to be treated.

Clause 22: The ophthalmic laser system of any one of the preceding clauses, wherein at least one of the body portions comprises at least a portion of a surrounding anatomy of an eyeball of at least one of the patient's eyes, in particular a lacrimal punctum and/or a lacrimal caruncle.

Clause 23: The ophthalmic laser system of any one of the preceding clauses, wherein the orientation-related data are acquired using at least one of the objects, wherein (a) at least one of the objects is in contact with and/or covers at least a portion of the untreated eye and/or (b) at least one of the objects is in contact with and/or covers at least a portion of the eye to be treated.

Clause 24: The ophthalmic laser system of any one of the preceding clauses, wherein the orientation analysis system comprises at least two imaging systems; wherein a first one of the imaging systems is configured to image (i) at least a portion of the eye to be treated; and/or (ii) at least a portion of an optical element, in particular a patient interface, which is in the beam path of the treatment laser beam between the beam delivery system and the eye of the patient; and wherein the second one of the imaging systems is configured to image at least a portion of the untreated eye and/or at least a portion of an object, which is in contact with and/or which covers at least a portion of the untreated eye.

Clause 25: The ophthalmic laser system of any one of the preceding clauses, wherein the orientation analysis system comprises an imaging system configured to image (i) at least a portion of the eye to be treated; and/or (ii) at least a portion of an optical element, in particular a patient interface, which is in the beam path of the treatment laser beam between the beam delivery system and the eye of the patient; wherein the orientation analysis system further comprises a system for acquiring a 3D image and/or a topographic representation of at least a portion of the untreated eye and/or an object, which is in contact with and/or which covers at least a portion of the untreated eye.

Clause 26: The ophthalmic laser system of any one of the preceding clauses, wherein one of the objects is at least a portion of an eye fixation device, in particular at least a portion of a suction ring.

Clause 27: The ophthalmic laser system of clause 26, wherein the controller is configured to determine, automatically or based on user input, a position and/or an orientation of the eye fixation device, in particular a position and/or an orientation of one or more marks, in particular infrared marks, of the eye fixation device based on the orientation-related data.

Clause 28: The ophthalmic laser system of any one of the preceding clauses, wherein the orientation analysis system comprises an aiming system for aiming at one or more of the body portions and/or objects along an aiming direction.

Clause 29: The ophthalmic laser system of clause 28, wherein (a) the aiming system comprises a light source for generating an aiming beam extending along the aiming direction; and/or (b) the aiming system comprises a mechanical indicator, which visibly indicates the aiming direction; and/or (c) the aiming system, comprises an array of light sources, which are arranged in a circumferential manner, wherein the aiming system is configured so that one or more active light sources of the array of light sources indicate the aiming direction.

Clause 30: The ophthalmic laser system of clause 28 or 29, wherein the aiming system is configured so that the aiming direction is adjustable by the user and/or controller.

Clause 31: An ophthalmic laser system having a beam delivery system, wherein the laser system is configured to perform laser treatments on an eye of a patient using a treatment laser beam, the laser system comprising: a beam delivery system for focusing the treatment laser beam so that the treatment laser beam travels toward the patient's eye; a controller operatively coupled with the beam delivery system and configured to control the beam delivery system to position a focus of the laser beam at different locations within the eye or on an anterior surface of the cornea of the eye; a guide configured to allow moving at least a portion of the beam delivery system to adjust an orientation of at least the movable portion to different orientations of the patient's head measured around an axis of the eye to be treated; an orientation indicating system which is configured to indicate, to the user, a relative orientation between (a) an orientation of a body portion of the patient and/or an object, which is in contact with and/or covers at least a portion of the body portion; and (b) an orientation of the movable portion of the beam delivery system, wherein the relative orientation is relative to an axis of the eye to be treated.

Clause 32: The laser system of clause 31, wherein the orientation indicating system comprises a light source, wherein (a) a position and/or an orientation of a light emitting surface of the light source; and/or (b) a direction of light emitted by the light source is indicative of or correlated with the orientation of the movable portion of the beam delivery system.

Clause 33: The laser system of clause 32, wherein the light source is a laser, in particular a line laser.

Clause 34: The laser system of any one of any one of clauses 31 to 33, wherein the orientation indicating system comprises an aiming system for aiming at one or more pre-defined body portions of the patient.

Clause 35: The laser system of any one of clauses 31 to 34, wherein the orientation indicating system comprises a light source, wherein light, which is emitted from the light source generates a visible mark on the patient's body of the patient or on an object, which is attached to and/or covers at least a portion of the patient's body.

Clause 36: The laser system of clause 35, wherein a position and/or an orientation of the visible mark is indicative of or correlates with the relative orientation.

Clause 37: The laser system of any one of clauses 31 to 36, wherein the orientation indicating system comprises a mechanical direction indicator, wherein the indicated direction is indicative of or correlates with the orientation of the movable portion of the beam delivery system.

Clause 38: The ophthalmic laser system of any one of the preceding clauses, further comprising a supporting arm, wherein at least the portion of the beam delivery system is displaceably supported by, or is part of, a free end of the supporting arm.

Clause 39: The ophthalmic laser system of clause 38, wherein a second end of the supporting arm, which is opposite to the free end is: (a) connected to a base of the laser system, which supports the supporting arm; and/or (b) comprises an interface for connecting the supporting arm to a further component at the second end of the supporting arm.

Clause 40: The ophthalmic laser system of clause 38 or 39, further comprising a laser source for generating the treatment laser beam; wherein a second end of the supporting arm, which is opposite to the free end is connected to a base of the laser system, which supports the supporting arm; and wherein the base houses at least a portion of the laser source.

Clause 41: The ophthalmic laser system of any one of clauses 38 to 40, wherein the supporting arm comprises one or more sensors which are operatively coupled with the controller, and which comprise one or more of: (a) a linear and/or an angular position sensors; and/or (b) an inclinometers and/or an accelerometer; wherein the controller is configured to determine one or more, or all parameters of a position and/or orientation of the laser applicator based on output of the one or more linear and/or angular sensors.

Clause 42: A method of operating an ophthalmic laser system having a beam delivery system, for performing laser treatments of an eye of a patient using a treatment laser beam, the laser system comprising: a beam delivery system for focusing the treatment laser beam so that the treatment laser beam travels toward the eye to be treated; a controller operatively coupled with the beam delivery system and configured to control the beam delivery system to position a focus of the laser beam at different locations within the eye or on an anterior surface of the cornea of the eye; a guide configured to allow moving at least a portion of the beam delivery system to adjust an orientation of at least the movable portion to different orientations of the patient's head measured around an axis of the eye to be treated; wherein the method comprises: acquiring, using an orientation analysis system, orientation-related data using one or more body portions of the patient and/or one or more objects, each of which being in a fixed position and/or orientation relative to a respective body portion so that the orientation-related data depend on an orientation of the one or more body portions around the axis of the eye; moving, by a user and/or a controller of the laser system, using the orientation-related data, the movable portion of the beam delivery system so that during the laser treatment, the movable portion has an orientation around the axis of the eye and relative to the patient's head, which corresponds or substantially corresponds to a pre-determined target orientation or which is within or substantially within a pre-determined target range.

Clause 43: A method of operating an ophthalmic laser system having a beam delivery system, for performing laser treatments of an eye of a patient using a treatment laser beam, the laser system comprising: a beam delivery system for focusing the treatment laser beam so that the treatment laser beam travels toward the eye to be treated; a controller operatively coupled with the beam delivery system and configured to control the beam delivery system to position a focus of the laser beam at different locations within the eye or on an anterior surface of the cornea of the eye; the method comprising: acquiring, using an orientation analysis system, orientation-related data using one or more body portions of the patient and/or one or more objects, each of which being in contact with and/or cover a respective body portion, so that the orientation-related data depend on an orientation of the one or more body portions around the axis of the eye; wherein at least one of the body portions and/or objects is outside the eye to be treated; wherein the controller is further configured to determine, based on the orientation-related data: (i) one or more parameters of the laser positioning data; and/or (ii) whether an exit optical element of the beam delivery system is positioned in front of the left or in front of the right eye of the patient.

Clause 44: The method of clause 42 or 43, wherein the orientation-related data are determined using one or more of the objects, wherein the method further comprises: placing, using the user and/or the ophthalmic laser system, at least one of the objects so that the object is in contact with and/or covers a body portion of the patient's body; and acquiring the orientation-related data at least from a portion of the placed objects.

Clause 45: The method of clause 44, wherein the placing of the object comprises placing the object on a further object, in particular on a portion of a tissue, which is in contact and/or which covers the body portion.

Clause 46: The method of clause 44 or 45, wherein the placed object is an anatomical portion of the surgeon, in particular a finger or a tip of the finger of the surgeon's hand.

Clause 47: A method of operating an ophthalmic laser system having a beam delivery system, wherein the laser system is configured to perform laser treatments on an eye of a patient using a treatment laser beam, the laser system comprising: a beam delivery system for focusing the treatment laser beam so that the treatment laser beam travels toward the patient's eye; a controller operatively coupled with the beam delivery system and configured to control the beam delivery system to position a focus of the laser beam at different locations within the eye or on an anterior surface of the cornea of the eye; a guide configured to allow moving at least a portion of the beam delivery system to adjust an orientation of at least the movable portion to different orientations of the patient's head measured around an axis of the eye to be treated; wherein the method comprises: indicating, using an orientation indicating system, to the user, a relative orientation between (a) an orientation of a body portion of the patient and/or an object, which is in (b) an orientation of the movable portion of the beam delivery system, wherein the orientation is relative to an axis of the eye to be treated.

Clause 48: The ophthalmic laser system of any one of clauses 1 to 30 and 38 to 41, wherein the orientation analysis system comprises a giga-terahertz imaging system for acquiring image data from the one or more body portions and/or objects; wherein the giga-terahertz imaging system is configured to use electromagnetic radiation having frequencies in a range of between 10 GHz and 100 THz.

Clause 49: An ophthalmic laser system for performing laser treatments of an eye using a treatment laser beam, the laser system comprising: a laser applicator, which comprises an optical system through which the treatment laser beam exits the laser applicator in a direction toward the patient's eye; a beam delivery system for focusing the treatment laser beam so that the treatment laser beam travels toward the eye to be treated; a supporting arm, wherein a free end of the supporting arm comprises or displaceably supports the laser applicator; a controller operatively coupled with the beam delivery system and configured to control the beam delivery system to position a focus of the laser beam at different locations within the eye or on an anterior surface of the cornea of the eye based on laser positioning data, which are indicative of or correlated with the locations within the eye to be treated; a giga-terahertz imaging system for acquiring image data from (a) one or more body portions of the patient; and/or (b) one or more objects, each of which having a fixed position and/or orientation relative to a respective body portion; wherein the giga-terahertz imaging system is configured to use electromagnetic radiation having frequencies in a range of between 10 GHz and 100 THz; wherein laser system is further configured to: (a) allow a user and/or the controller to adjust a relative position and/or orientation of the laser applicator relative to the patient's head using the image data; and/or (b) determine, using the controller, the laser positioning data based on the imaging data.

Clause 50: The ophthalmic laser system of clause 48 or 49, wherein the electromagnetic radiation has frequencies in a range of between 30 GHz and 50 THz.

Clause 51: The ophthalmic laser system of any one of clause 48 to 50, wherein terahertz and/or gigahertz imaging system comprises a scanner for scanning a beam of the electromagnetic radiation across the one or more body portions and/or objects.

Clause 52: The ophthalmic laser system of any one of clauses 48 to 51, wherein the controller is configured to determine, based on the image data, an outer contour of at least a portion of the patient's body.

Clause 53: The ophthalmic laser system of any one of clauses 48 to 52, wherein the free end of the supporting arm includes or displaceably supports at least a portion of the giga-terahertz imaging system.

Clause 54: The ophthalmic laser system of any one of clauses 48 to 53, wherein the controller is configured to determine, automatically or using user input, based on the image data, one or more parameters, which are (a) indicative of or (b) correlated with one or more, or all, parameters of an orientation of the patient's head around the axis of the eye to be treated.

Clause 55: The ophthalmic laser system of any one of clauses 48 to 54, further comprising: a drive system for driving a movement of the positioning arm to displace the laser applicator; wherein the controller is operatively coupled with the drive system for controlling the movement of the positioning arm based on the image data.

Clause 56: The ophthalmic laser system of any one of clauses 48 to 55, wherein a second end of the supporting arm, which is opposite to the free end is: (a) connected to a base of the laser system, which supports the supporting arm; and/or (b) comprises an interface for connecting the supporting arm to a further component at the second end of the supporting arm.

Clause 57: The ophthalmic laser system of clause 56, further comprising a laser source for generating the treatment laser beam; wherein a second end of the supporting arm, which is opposite to the free end is connected to a base of the laser system, which supports the supporting arm; and wherein the base houses at least a portion of the laser source.

Clause 58: The ophthalmic laser system of any one of clauses 48 to 57, wherein the treatment laser beam is a pulsed laser beam having pulse durations between 1 femtosecond and 1,000 femtoseconds.

Clause 59: An ophthalmic laser system for performing laser treatments of an eye, the laser system comprising: a laser applicator, which comprises an optical system through which the treatment laser beam exits the laser applicator in a direction toward the patient's eye; a supporting arm; and a controller; wherein a free end of the supporting arm includes the laser applicator or displaceably supports the laser applicator; wherein the supporting arm comprises a controllable visual indicator, which is in operative connection with the controller to control generation of a visual signal; wherein the visual indicator comprises: (a) a curved surface, from which signal light of the visual signal emanates, wherein the curved surface forms at least a circumferential portion of an outer circumferential surface of the supporting arm; and/or (b) a plurality of surfaces, wherein from each of the surfaces, signal light of the visual signal emanates, wherein the plurality of surfaces are distributed at least around a circumferential portion of the outer circumferential surface of the supporting arm.

Clause 60: The ophthalmic laser system of clause 59, wherein the circumferential surface has a horizontal or a substantially horizontal circumference.

Clause 61: The ophthalmic laser system of clause 59 or 60, wherein the supporting arm comprises two arm segments, wherein the two arm segments are connected via one or more joints, wherein the visual indicator is arranged at the one or more joints.

Clause 62: The ophthalmic laser system of clause 61, wherein a first arm segment of the two arm segments is rotatable about a vertical or substantially vertical axis and the second arm segment of the two arm segments is rotatable about a horizontal or substantially horizontal axis.

Clause 63: The ophthalmic laser system of clause 61 or 62, wherein the second arm segment is rotatable about a vertical axis in order to adjust an orientation of the second arm segment relative to the first arm segment.

Clause 64: The ophthalmic laser system of any one of clauses 62 or 63, wherein the first arm segment is connected to the laser applicator via the second arm segment.

Clause 65: The ophthalmic laser system of any one of clauses 59 to 64, wherein the controller is configured so that the visual signal light indicates a stage of the laser treatment.

Clause 66: The ophthalmic laser system of any one of clauses 59 to 65, wherein the controller is configured so that the visual signal indicates an operational state of the of ophthalmic laser system.

Clause 67: The ophthalmic laser system of any one of clauses 59 to 66, wherein the controller and the visual indicator are configured so that the visual signal indicates a warning message to a user.

Clause 68: The ophthalmic laser system of any one of clauses 59 to 67, wherein the supporting arm is configured so that the laser applicator is positionable relative to the base in three dimensions.

Clause 69: The ophthalmic laser system of any one of clauses 59 to 68, wherein the visual indicator comprises one or more emitting diodes (LED).

Clause 70: The ophthalmic laser system of any one of clauses 59 to 69, wherein the visual indicator comprises a plurality of light sources, wherein each of the light sources includes or is covered by one of the surfaces from which signal light emanates and which are distributed around at least the circumferential portion.

Clause 71: The ophthalmic laser system of any one of clauses 59 to 70, wherein the visual indicator includes a light reflector which is illuminated by one or more light sources of the visual indicator and which is configured for specular or diffuse reflection of signal light, which is emitted from the one or more light sources.

Clause 72: The ophthalmic laser system of any one of clauses 1 to 41 and 48 to 71, wherein the laser system comprises: a base, which houses at least a portion of a laser source of the laser system wherein the laser source is configured for generating a treatment laser beam for performing the laser treatments; a laser applicator, which comprises an optical system through which the treatment laser beam exits the laser applicator in a direction towards the patient's eye; a supporting arm; and a controller; wherein the supporting arm is connected to the laser applicator at a first end of the supporting arm, and wherein a second end of the supporting arm (a) is connected to the base and/or (b) comprises an interface for connecting the supporting arm to a further component at the second end of the supporting arm; wherein the supporting arm is configured so that the laser applicator is positionable relative to the base in three dimensions while maintaining a vertical orientation of the laser applicator; and wherein the laser system further comprises a motorized three-axis positioning system, which is operatively coupled to the controller for positioning the laser applicator relative to at least a portion of the supporting arm in three dimensions.

Clause 73: The laser system of clause 72, wherein laser applicator comprises a manually operable control unit which is operatively coupled to the controller for performing the positioning of the laser applicator relative to the supporting arm based on user input received via the control element.

Clause 74: The laser system of clauses 72 or 73, wherein the laser applicator comprises an imaging system for acquiring a frontal image of at least a portion of the patient's eye at least during part of the positioning of the laser applicator relative to the supporting arm.

Clause 75: The laser system of clause 74 wherein the laser applicator comprises a display device for displaying the frontal image at least during part of the positioning of the laser applicator relative to the supporting arm.

Clause 76: The laser system of any one of the preceding clauses 72 to 75, wherein the laser applicator comprises an interaction measuring unit for generating an output signal which depends on a parameter of a mechanical interaction between the patient's eye and the laser applicator.

Clause 77: The laser system of clause 76, wherein the output signal depends on a force between the patient's eye and the laser applicator; and/or the interaction determining unit includes a force sensor, a strain gauge sensor and/or a piezoelectric element.

Clause 78: The laser system of clause 76 or 77, wherein the laser applicator comprises a display device, wherein the controller is configured to generate data representative of graphical and/or textual information using the output signal generated by the interaction measuring unit; and to display the graphical and/or textual information on the display device at least during part of the positioning of the laser applicator relative to the supporting arm.

Clause 79: The laser system of any one of the preceding clauses 1 to 41 and 48 to 78, wherein the laser applicator comprises an optical coherence tomography (OCT) system which is configured for acquiring a cross-sectional image of at least a portion of the eye.

Clause 80: The laser system of clause 79, wherein the laser applicator comprises a beam combiner for combining a beam path of a measuring arm of the OCT system with a beam path of the treatment laser beam.

Clause 81: The laser system of any one of the preceding clauses 1 to 41 and 48 to 80, wherein the supporting am comprises an arm segment which is rotatable about a horizontal or substantially horizontal axis.

Clause 82 The laser system of clause 81, wherein the arm segment comprises a parallel linkage mechanism.

Clause 83: The laser system of any one of the preceding clauses 1 to 41 and 48 to 82, wherein the supporting arm comprises a first arm segment and a second arm segment which are connected to each other in series via an intermediate joint; wherein the first arm segment is rotatable about a vertical or substantially vertical axis and the second arm segment is rotatable about a horizontal or substantially horizontal axis.

Clause 84: The laser system of clause 83, wherein the intermediate joint is configured to allow the second arm segment to rotate about the horizontal or substantially horizontal axis and about a vertical or substantially vertical axis.

Clause 85: The laser system of clause 83 or 84, wherein the second arm segment comprises a parallel linkage.

Clause 86: The laser system of any one of the preceding clauses 1 to 41 and 48 to 85, wherein the supporting arm comprises a counterbalancing mechanism for providing gravity counterbalancing for the applicator head.

Clause 87: The laser system of clause 86, wherein the supporting arm comprises one or more springs, wherein the supporting arm is configured to provide at least a portion of the gravity counterbalancing using the one or more springs.

Clause 88: The laser system of any one of the preceding clauses 1 to 41 and 48 to 87, wherein the supporting arm comprises a braking and/or locking system for arresting a movement of the second end of the supporting arm relative to the first end of the supporting arm.

Clause 89: The laser system of clause 88, wherein the laser applicator comprises a manually operable control unit for selectively activating and deactivating the braking and/or locking system based on user input received via the control unit.

Clause 90: The laser system of clause 88 or 89, wherein the laser system comprises an interaction measuring unit configured for generating an output signal which depends on a mechanical interaction between the patient's eye and the laser applicator; wherein the controller is operatively connected to the interaction measuring unit and the braking and/or locking system; wherein the controller is configured to receive the output signal generated by the interaction measuring unit and to determine based on the received output signal, whether or not to deactivate the braking and/or locking system.

Clause 91: The laser system of any one of the preceding clauses 1 to 41 and 48 to 90, further comprising an articulated beam guide tube, wherein at least a portion of the articulated beam guiding tube extends between a first location on or within the base or an arm segment of the supporting arm and a second location on or within the laser applicator.

Clause 92: The laser system of any one of the preceding clauses 1 to 41 and 48 to 91, wherein the supporting arm has a rotational joint, which has a vertically extending rotation axis for rotating the laser applicator about an axis which extends through the laser applicator.

Clause 93: The laser system of clause 92, wherein the laser system comprises a locking system which is configured for locking the rotational joint having the rotation axis, which extends through the laser applicator.

Clause 94: The laser system of any one of the preceding clauses 1 to 41 and 48 to 93, wherein the laser applicator comprises: an objective lens for focusing the treatment laser beam within the eye and/or an axial scanning system for scanning the laser focus along an axis of the laser beam; and/or a beam deflection scanning system for scanning the laser beam through deflection of the laser beam.

Clause 1A: An ophthalmic laser system for performing laser treatments of an eye, the laser system comprising: a base, which houses at least a portion of a laser source of the laser system wherein the laser source is configured for generating a treatment laser beam for performing the laser treatments; a laser applicator, which comprises an optical system through which the treatment laser beam exits the laser applicator in a direction towards the patient's eye; a supporting arm; and a controller; wherein the supporting arm is connected to the laser applicator at a first end of the supporting arm, and wherein a second end of the supporting arm (a) is connected to the base and/or (b) comprises an interface for connecting the supporting arm to a further component at the second end of the supporting arm; wherein the supporting arm is configured so that the laser applicator is positionable relative to the base in three dimensions while maintaining a vertical orientation of the laser applicator; and wherein the laser system further comprises a motorized three-axis positioning system, which is operatively coupled to the controller for positioning the laser applicator relative to at least a portion of the supporting arm in three dimensions.

Clause 2A: The laser system of clause 1A, wherein laser applicator comprises a manually operable control unit which is operatively coupled to the controller for performing the positioning of the laser applicator relative to the supporting arm based on user input received via the control element.

Clause 3A: The laser system of clauses 1A or 2A, wherein the laser applicator comprises an imaging system for acquiring a frontal image of at least a portion of the patient's eye at least during part of the positioning of the laser applicator relative to the supporting arm.

Clause 4A: The laser system of clause 3A, wherein the laser applicator comprises a display device for displaying the frontal image at least during part of the positioning of the laser applicator relative to the supporting arm.

Clause 5A: The laser system of any one of the preceding clauses 1A to 4A, wherein the laser applicator comprises an interaction measuring unit for generating an output signal which depends on a parameter of a mechanical interaction between the patient's eye and the laser applicator.

Clause 6A: The laser system of clause 5A, wherein the output signal depends on a force between the patient's eye and the laser applicator; and/or the interaction determining unit includes a force sensor, a strain gauge sensor and/or a piezoelectric element.

Clause 7A: The laser system of clause 5A or 6A, wherein the laser applicator comprises a display device, wherein the controller is configured to generate data representative of graphical and/or textual information using the output signal generated by the interaction measuring unit; and to display the graphical and/or textual information on the display device at least during part of the positioning of the laser applicator relative to the supporting arm.

Clause 8A: An ophthalmic laser system for performing laser treatments of an eye, the laser system comprising: a base, which houses at least a portion of a laser source of the laser system wherein the laser source is configured for generating a treatment laser beam for performing the laser treatments; a laser applicator, which comprises an optical system through which the treatment laser beam exits the laser applicator towards the patient's eye; a supporting arm, which is connected to the laser applicator at a first end of the supporting arm and wherein the supporting arm (a) is connected to the base and/or (b) comprises an interface for connecting the supporting arm to a further component at a second end of the supporting arm; an articulated beam guide tube, wherein at least a portion of the articulated beam guide tube extends between a first location where the treatment laser beam exits from the base or the supporting arm and a second location, where the treatment laser beam enters into the supporting arm or the laser applicator; wherein laser system includes a coupling arrangement for coupling the articulated beam guiding tube to the supporting arm at one or more locations along the beam guiding tube, between the first location and the second location.

Clause 9A: The laser system of clause 8A, wherein the coupling arrangement comprises a tensile force transmitting connection.

Clause 10A: The laser system of clause 9A, wherein the tensile force transmitting connection comprises a tension spring for transmitting the tensile force.

Clause 11A: The laser system of any one of clauses 8A to 10A, wherein the coupling arrangement comprises a guide, in particular a lateral guide.

Clause 12A: The laser system of clause 11A, wherein the guide is configured to limit, during a movement of the laser applicator, a variation of a vertical orientation of a plane defined by consecutive arm segments of the articulated beam guide tube.

Clause 13A: The laser system of any one of the preceding clauses 1A to 12A, wherein the laser applicator comprises an optical coherence tomography (OCT) system which is configured for acquiring a cross-sectional image of at least a portion of the eye.

Clause 14A: The laser system of clause 13A, wherein the laser applicator comprises a beam combiner for combining a beam path of a measuring arm of the OCT system with a beam path of the treatment laser beam.

Clause 15A: The laser system of any one of the preceding clauses 1A to 14A, wherein the supporting am comprises an arm segment which is rotatable about a horizontal or substantially horizontal axis.

Clause 16A: The laser system of clause 15A, wherein the arm segment comprises a parallel linkage mechanism.

Clause 17A: The laser system of any one of the preceding clauses 1A to 16A, wherein the supporting arm comprises a first arm segment and a second arm segment which are connected to each other in series via an intermediate joint; wherein the first arm segment is rotatable about a vertical or substantially vertical axis and the second arm segment is rotatable about a horizontal or substantially horizontal axis.

Clause 18A: The laser system of clause 17A, wherein the intermediate joint is configured to allow the second arm segment to rotate about the horizontal or substantially horizontal axis and about a vertical or substantially vertical axis.

Clause 19A: The laser system of clause 17A or 18A, wherein the second arm segment comprises a parallel linkage.

Clause 20A: The laser system of any one of the preceding clauses 1A to 19A, wherein the supporting arm comprises a counterbalancing mechanism for providing gravity counterbalancing for the applicator head.

Clause 21A: The laser system of clause 20A, wherein the supporting arm comprises one or more springs, wherein the supporting arm is configured to provide at least a portion of the gravity counterbalancing using the one or more springs.

Clause 22A: The laser system of any one of the preceding clauses 1A to 21A, wherein the supporting arm comprises a braking and/or locking system for arresting a movement of the second end of the supporting arm relative to the first end of the supporting arm.

Clause 23A: The laser system of clause 22A, wherein the laser applicator comprises a manually operable control unit for selectively activating and deactivating the braking and/or locking system based on user input received via the control unit.

Clause 24A: The laser system of clause 22A or 23A, wherein the laser system comprises an interaction measuring unit configured for generating an output signal which depends on a mechanical interaction between the patient's eye and the laser applicator; wherein the controller is operatively connected to the interaction measuring unit and the brakes; wherein the controller is configured to receive the output signal generated by the interaction measuring unit and to determine based on the received output signal, whether or not to deactivate the braking and/or locking system.

Clause 25A: The laser system of any one of the preceding clauses 1A to 24A, further comprising an articulated beam guide tube, wherein at least a portion of the articulated beam guiding tube extends between a first location on or within the base or an arm segment of the supporting arm and a second location on or within the laser applicator.

Clause 26A: The laser system of any one of the preceding clauses 1A to 25A, wherein the supporting arm has a rotational joint, which has a vertically extending rotation axis for rotating the laser applicator about an axis which extends through the laser applicator.

Clause 27A: The laser system of clause 25A or 26A, wherein the laser system comprises a locking system which is configured for locking the rotational joint having the rotation axis, which extends through the laser applicator.

Clause 28A: The laser system of any one of the preceding clauses 1A to 27A, wherein the laser applicator comprises: an objective lens for focusing the treatment laser beam within the eye and/or an axial scanning system for scanning the laser focus along an axis of the laser beam; and/or a beam deflection scanning system for scanning the laser beam through deflection of the laser beam.

Clause 29A: A method of positioning a laser applicator of an ophthalmic laser system relative to a patient's eye, the method comprising: positioning a laser applicator relative to the patient's eye using a supporting arm, wherein the supporting arm is connected to a base of the ophthalmic laser system at a first end of the supporting arm; and wherein the supporting arm is connected to the base and/or connectable to a stationary component at a second end of the supporting arm; wherein base houses at least a portion of a laser source of the laser system wherein the laser source is configured for generating a treatment laser beam for performing the laser treatments; wherein the supporting arm is configured so that the laser applicator is positionable relative to the base base while maintaining a vertical orientation of the laser applicator; and positioning the laser applicator relative to at least a portion of the supporting arm using a motorized three-axis positioning system.

Clause 30A: The method of clause 29A, further comprising: acquiring a frontal image of the eye using an imaging system of the laser applicator; and displaying, during at least part of the positioning of the laser applicator relative to the supporting arm, the frontal image on a display device of the laser system.

Clause 31A: The method of clause 30A, wherein during the part of the positioning, a distance of the focal plane from the laser applicator substantially corresponds to a pre-defined distance of the laser applicator from the patient's eye.

Clause 32A: The method of any one of clause 29A to 31A, further comprising: generating, by an interaction measuring unit, an output signal, which depends on a mechanical interaction between the patient's eye and the laser applicator; determining, using a controller of the laser system, textual and/or graphical information based on the output signal; and displaying, at least during part of the positioning of the laser applicator relative to the supporting arm, the textual and/or graphical information.

Clause 33A: The method of any one of clauses 29A to 32A, further comprising: generating, by an interaction measuring unit, an output signal, which depends on a mechanical interaction between the patient's eye and the laser applicator; determining, using a controller of the laser system and based on the output signal, whether or not to deactivate brakes of the supporting arm, which arrest a movement of the second end of the supporting arm relative to the first end of the supporting arm.

Clause 34A: The method of any one of clauses 29A to 33A, wherein the output signal depends on a force between the patient's eye and the laser applicator; and/or the interaction determining unit includes a force sensor and/or a strain gauge sensor.

Clause 35A: The laser system of any one of clauses 1A to 7A, further comprising an articulated beam guide tube, wherein at least a portion of the articulated beam guide tube extends between a first location where the treatment laser beam exits from the base or the supporting arm and a second location, where the treatment laser beam enters into the supporting arm or the laser applicator; wherein laser system includes a coupling arrangement for coupling the articulated beam guiding tube to the supporting arm at one or more locations along the beam guiding tube, between the first location and the second location.

Clause 36A: The laser system of clause 35A, wherein the coupling arrangement comprises a a tensile force transmitting connection.

Clause 37A: The laser system of clause 36A, wherein the tensile force transmitting connection comprises a tension spring for transmitting the tensile force.

Clause 38A: The laser system of any one of clauses 35A to 37A, wherein the coupling arrangement comprises a guide, in particular a lateral guide.

Clause 39A: The laser system of clause 38A, wherein the guide is configured to limit, during a movement of the laser applicator, a variation of a vertical orientation of a plane defined by consecutive arm segments of the articulated beam guide tube.

We claim:

1. An ophthalmic laser system for performing laser treatments of an eye, the laser system comprising:
a laser applicator, which comprises an optical system through which a treatment laser beam exits the laser applicator in a direction toward the eye;
a supporting arm; and a controller; wherein a free end of the supporting arm includes the laser applicator or displaceably supports the laser applicator;
wherein the supporting arm comprises a controllable visual indicator, which is in operative connection with the controller to control generation of a visual signal; wherein the visual indicator comprises:
(a) a curved surface, from which signal light of the visual signal emanates, wherein the curved surface forms at least a circumferential portion of an outer circumferential surface of the supporting arm; and/or
(b) a plurality of surfaces, wherein from each of the surfaces, signal light of the visual signal emanates, wherein the plurality of surfaces are distributed at least around a circumferential portion of the outer circumferential surface of the supporting arm.

2. The ophthalmic laser system of claim 1, wherein the circumferential surface has a horizontal circumference.

3. The ophthalmic laser system of claim 1, wherein the supporting arm comprises two arm segments, wherein the two arm segments are connected via one or more joints, wherein the visual indicator is arranged at the one or more joints.

4. The ophthalmic laser system of claim 3, wherein one of the two arm segments is rotatable about a vertical axis in order to adjust an orientation of a first arm segment of the two arm segments relative to a second arm segment of the two arm segments.

5. The ophthalmic laser system of claim 3, wherein a first arm segment of the two arm segments is rotatable about a vertical axis and the second arm segment of the two arm segments is rotatable about a horizontal axis.

6. The ophthalmic laser system of claim 5, wherein the first arm segment is connected to the laser applicator via the second arm segment.

7. The ophthalmic laser system of claim 1, further comprising a laser source for generating the treatment laser beam;
wherein a second end of the supporting arm, which is opposite to the free end is connected to a base of the laser system, which supports the supporting arm; and wherein the base houses at least a portion of the laser source.

8. The ophthalmic laser system of claim 7, wherein the supporting arm is configured such that the laser applicator is positionable relative to the base in three dimensions while maintaining a vertical orientation of the laser applicator.

9. The ophthalmic laser system of claim 1, wherein the supporting arm comprises a first arm segment which is connected to a second arm segment of the supporting arm via an intermediate joint,
wherein the visual indicator is arranged at the intermediate joint.

10. The ophthalmic laser system of claim 9, wherein the intermediate joint is configured such that an orientation of the second arm segment relative to the first arm segment is adjustable in two rotary degrees of freedom.

11. The ophthalmic laser system of claim 1, wherein the visual indicator is mounted to or is part of an arm segment of the supporting arm.

12. The ophthalmic laser system of claim 1, wherein the visual indicator comprises the curved surface, wherein the circumferential portion represents more than 50% of the circumference of the circumferential surface.

13. The ophthalmic laser system of claim 1, wherein the visual indicator comprises the curved surface,
wherein the curved surface is formed from a cylindrical cover, which covers one or more light sources of the visual indicator.

14. The ophthalmic laser system of claim 1, wherein the controller is configured so that the visual signal indicates a stage of the laser treatment.

15. The ophthalmic laser system of claim 1, wherein the controller is configured so that the visual signal indicates an operational state of the ophthalmic laser system.

16. The ophthalmic laser system of claim 1, wherein the controller and the visual indicator are configured so that the visual signal indicates a warning message to a user.

17. The ophthalmic laser system of claim 1, wherein the supporting arm is configured so that the laser applicator is positionable relative to a base of the laser system.

18. The ophthalmic laser system of claim 1, wherein the visual indicator comprises one or more emitting diodes (LED).

19. The ophthalmic laser system of claim 1, wherein the visual indicator comprises a plurality of light sources, wherein each of the light sources includes or is covered by one of the surfaces from which signal light emanates and which are distributed around at least the circumferential portion.

20. The ophthalmic laser system of claim 1, wherein the visual indicator includes a light reflector which is illuminated by one or more light sources of the visual indicator and which is configured for specular or diffuse reflection of signal light, which is emitted from the one or more light sources.
